(19) United States
(12) Patent Application Publication
(10) Pub. No.: US 2013/0177528 A1
(43) Pub. Date: Jul. 11, 2013

(54) COMPOSITIONS AND METHODS FOR INHIBITING VIRAL INFECTION

(71) Applicants: University of Southern California, Los Angeles, CA (US); Western University of Health Sciences, Pomona, CA (US)

(72) Inventors: Francis Markland, Manhattan Beach, CA (US); Radu Minea, Arcadia, CA (US); Stephen Swenson, Glendale, CA (US); Vaibhav Tiwari, Pomona, CA (US)

(73) Assignees: WESTERN UNIVERSITY OF HEALTH SCIENCES, Pomona, CA (US); UNIVERSITY OF SOUTHERN CALIFORNIA, Los Angeles, CA (US)

(21) Appl. No.: 13/674,827

(22) Filed: Nov. 12, 2012

Related U.S. Application Data
(60) Provisional application No. 61/558,388, filed on Nov. 10, 2011.

(51) Int. Cl. A61K 38/17; A61K 45/06; A61K 38/44; C12Q 1/02; A61K 38/48

(52) U.S. Cl. CPC A61K 38/1703; C12Q 1/025; A61K 38/4886; A61K 38/44; A61K 45/06
USPC 424/85.4; 435/375; 514/3.7; 424/94.67; 424/94.4; 514/4.2; 424/204.1; 435/5; 530/350; 435/219

(57) ABSTRACT

Provided herein are compositions and methods for inhibiting viral infection of a host cell. The methods comprise contacting the the host cell with an effective amount of one or more polypeptides having a disintegrin domain. The polypeptide can be CN, VCN or modified ADAM-derived polypeptide (MAP), or a fusion protein comprising a CN, VCN or MAP.

### CROSS-REFERENCE TO RELATED APPLICATIONS

This application claims priority under 35 U.S.C. §119(e) to U.S. Provisional Application Ser. No. 61/558,388, filed Nov. 10, 2011, the contents of which are incorporated herein by reference in their entirety.

### FIELD OF INVENTION

The invention relates to methods for the prevention and treatment of viral infection.

### BACKGROUND OF THE INVENTION

Vaccines to prevent viral infection and antiviral drugs that inhibit or slow the infection process are available for only a few virus-borne diseases and few are fully effective in preventing or treating a viral infection Inhibition of viral infection by viral entry inhibitors—stopping transmission at the gateway of the cell—is an attractive tool in the anti-infectives armament. Infection by viruses having lipid-bilayer envelopes proceeds through fusion of the viral membrane with a membrane of the target cell. Viral ‘fusion proteins’ facilitate this process. Viral entry inhibitors inhibit protein—protein interactions either within the viral envelope (Env) glycoproteins or between viral Env and host-cell receptors. In addition, the nature of resistance to entry inhibitors differs from compounds that inhibit enzymatic targets due to their different modes of action and the variability of Env amino acid sequences, both temporally and among patients.

For example, the antiretroviral drug, Fuzeon® binds to Human Immuno Deficiency (HIV) gp41 and interferes with its ability to approximate the membranes of the virus and host cell. Fuzeon® is in a class of viral entry inhibitors, also known as fusion inhibitors, which are used in combination therapies to treat HIV infection. This class of drugs interfere with the binding, fusion and entry steps of an HIV virion's infection of a cell. By blocking this step in HIV's replication cycle, such drugs slow the progression from HIV infection to Acquired Immune Deficiency Syndrome (AIDS).

There exists a need for improved anti-infectives of HIV and other viruses.

Herpes simplex virus type-1 (HSV-1) infection is common among humans, and is typically associated with outbreaks of facial cold sores. Recurrent infections in the eye can cause corneal blindness [1]. Severe complications, especially in neonates and immunocompromised patients, may result in retinitis and inflammation in the brain tissues that lead to encephalitis [2-6]. HSV-1 virions infect host cells by initially attaching to cell surface heparan sulfate (HS), followed by fusion of the virion envelope with the plasma membrane of the host cells [7, 8]. The current model of HSV-1 infection suggests that entry of the virion requires four HSV glycoproteins, glycoprotein (g)B, (g)D, (g)H and (g)L [9], and at least one cellular receptor for gD [10, 11]. Receptors for HSV-1 gD include a member of the TNF-receptor family named HVEM [12], a member of the immunoglobulin superfamily commonly known as nectin-1 [13] and HS proteoglycans that have been modified by various D-glucosaminyl 3-O-sulfotransferase (3-OST) isoforms. Among the known 3-OST isoforms, all but one isoform, 3-OST-1, mediate HSV-1 entry [14] and cell-to-cell fusion [11]. Paired immunoglobulin-like type 2 receptor-alpha (PILR-α) may also serve as co-receptor for HSV-1 through its interaction with gB [15].

Human cytomegalovirus (HCMV) asymptomatically infects humans throughout the world at a high rate of incidence. Primary infection is followed by a life-long latent phase that may reactivate and cause disease; for example, in immunosuppressed individuals, such as AIDS patients and organ transplant recipients [16, 17]. Primary congenital HCMV infections are also a cause of significant morbidity and mortality [18]. Currently, there is no effective HCMV vaccine, and HCMV antiviral drugs, such as ganciclovir, are highly toxic and unsuitable for treating pregnant women in the congenital setting [19].

HCMV disease can manifest itself in most organ systems and tissue types. Pathology from HCMV-infected individuals reveals that HCMV can infect most cell types, including fibroblasts, endothelial cells, epithelial cells, smooth muscle cells, stromal cells, monocytes/macrophages, neutrophils, neuronal cells, and hepatocytes [20-24]. The broad intrahost organ and tissue tropism of HCMV is paralleled in vitro with the virus' ability to bind and fuse with nearly every vertebrate cell type tested [25-27]. However, full productive infection is limited to secondary strains of fibroblasts and endothelial cells. The ability of HCMV to enter such a diverse range of cell types is indicative of multiple cell-specific receptors, broadly expressed receptors, or a complex entry pathway in which a combination of both cell-specific and broadly expressed cellular receptors are utilized.

Throughout the Herpesviridae [29], the genes that encode envelope gB and gH are essential for viral infection [28], play several key roles during virus entry and egress, and are conserved [29]. A soluble form of gB (gB-s), which is truncated at its transmembrane domain can bind specifically to permissive cells; thus allowing it to block virus entry, and trigger signal transduction events that result in the activation of an interferon-responsive pathway that is also activated by HCMV virions [30-32].

Entry of HCMV into cells requires initial tethering of virions to cell surface heparan sulfate proteoglycans (HSPGs) [33, 29]. The HCMV envelope contains at least two separate glycoprotein complexes with affinities for HS, gB [22] and the gM/gN complex [34]. The gM/gN complex is more abundant than gB on the HCMV envelope, and it binds heparin with higher affinity [35]. Thus, the gM/gN complex is thought to be the primary heparin-binding component of the HCMV envelope.

Integrins are heterodimers composed of non-covalently associated alpha and beta submits. Interactions between integrins and extra cellular matrix (ECM) proteins have been shown to be mediated via the ECM tripeptide sequence, Arg-Gly-Asp (RGD). Both the alpha and beta subunits of the integrin are required for ECM protein binding.

Disintegrins are a family of snake venom proteins that include those from venom of Crotalidae and Viperidae families of snakes. They inhibit integrin-ECM interactions and glycoprotein (GP) IIb/IIIa mediated platelet aggregation. Disintegrins are disulfide rich, and many family members contain an exposed RGD sequence that is located at the tip of a flexible loop termed the integrin-binding loop. This loop is stabilized by disulfide bonds and protrudes from the main body of the polypeptide chain. The RGD sequence of the integrin-binding loop enables a polypeptide comprising a disintegrin domain to bind to integrins with high affinity.

Polypeptides which comprise a disintegrin domain, and are known to disrupt integrin interactions, include: bitistatin (83 amino acids), a disintegrin isolated from the venom of Bitis arietans; echistatin (49 amino acids), a disintegrin isolated from the venom of Echis cannatus; kistrin (68 amino acids), a disintegrin isolated from the venom of Calloselasma rhodostoma; trigamin (72 amino acids), a disintegrin isolated from the venom of Trimeresurus gramineus; applaggin, which is isolated from the venom of Agkistrodon piscivorus piscivorus; and contortrostatin (CN), which is isolated from the venom of Agkistrodon contortix contortix (the southern copperhead snake).

With respect to CN, its full-length precursor cDNA has been cloned and sequenced [36]. The sequence can be accessed in the GenBank database using accession number AF212305. CN is produced in the snake venom gland as a 2027 bp multidomain precursor with a 1449 bp open reading frame encoding a precursor polypeptide (shown below) that includes a pro-protein domain (amino acids 1 to 190), a metalloproteinase domain (amino acids 191 to 410), and a disintegrin domain (amino acids 419 to 483):

| | |
|---|---|
| 1 | miqvllvtlc laafpyqgss iilesgnvnd yevlypqkvt alpkgavqpk yedtmqyefk |
| 61 | vngepvvlhl eknkglfskd ysethyssdg rkittnppve dhcyyhgriq ndadstasis |
| 121 | acnglkghfk lqgetyliep lklsdseaha vykyenveke deapkmcgvt qtnwesdepi |
| 181 | kkasqlnltp eqqgfpqryi elvvvadhrm ftkyngnlnt iriwvhelvn tmnvfyrpln |
| 241 | irvsltdlev wsdqdlinvq paaadtleaf gdwretvlln rishdnaqll taieldgeti |
| 301 | glanrgtmcd pklstgivqd hsainlwvav tmahemghnl gishdgnqch cdanscimse |
| 361 | elreqlsfef sdcsqnqyqt yltdhnpqcm lneplrtdiv stpvsgnell etgeesdfda |
| 421 | panpccdaat cklttgsqca dglccdqckf mkegtvcrra rgddlddycn gisagcprnp |
| 481 | fha |

Identified receptors of CN include integrins αIIbβ3, αvβ3, αvβ5, and α5β1.

U.S. Pat. No. 7,754,850, issued Jul. 13, 2010 describes vicrostatin (VCN), a recombinant fusion protein wherein the last three amino acids of the carboxy terminus of CN are swapped with the C-terminal tail of echistatin, which has the amino acid sequence HGKPAT. VCN can be expressed using the Origami® B (DE3)/pET32a system (EMD4Biosciences, Merck KGaA, Darmstadt, Germany). Unlike other E. coli strains, Origami® B is unique in that, by carrying mutations in thioredoxin reductase (trxB) and glutathione reductase (gor), two key genes that are critically involved in the control of the two major oxido-reductive pathways in E. coli. These mutations result in a bacterium cytoplasmic microenvironment that is artificially shifted to a more oxidative redox state, which acts as a catalyst environment for disulfide bridge formation in proteins. An improved method of expression of VCN is disclosed in International Patent Publication No. WO 2010/056901.

Other polypeptides that have a disintegrin domain include those of the ADAM (A Disintegrin and Metalloproteinase) family. There are over 30 ADAM proteins indentified in the mammalian kingdom, and all of them have a disintegrin domain. Humans possess 20 ADAM genes and three ADAM pseudogenes.

Coulson et al. [53] state that they identified a novel “disintegrin-like” domain, in Rotavirus capsid protein VP7 by comparing it to disintegrins, the disintegrin-like domain of snake venom metalloproteinases and members of the ADAM gene family. Coulson et al. also report that certain very short (3-6 amino acid) peptides from this domain can reduce Rotavirus infectivity. Likewise, Feire et al. [54] state that they identified a “disintegrin-like” domain in viral fusion glycoprotein HCMV gB and that peptides derived from this region of gB inhibit HCMV viral infectivity.

### SUMMARY OF THE INVENTION

Provided herein are methods of inhibiting viral infection of a host cell by administering to the host cell an effective amount of a polypeptide having a disintegrin domain or a mixture of polypeptides having at least one disintegrin domain. The polypeptide having a disintegrin domain include, for example, CN, VCN or modified ADAM-derived polypeptide (MAP), or a fusion protein comprising a CN, VCN or MAP. The fusion can be with thioredoxin A or a fragment thereof. The MAP can be MAP1, MAP2, MAP3, MAP6, MAP7, MAP8, MAP9, MAP10, MAP11, MAP12, MAP15, MAP17, MAP18, MAP19, MAP20, MAP21, MAP22, MAP23, MAP28, MAP29, MAP30, MAP32, MAP33. The viral infection can be by a virus that uses a host cell integrin as a receptor for infection.

Also provided herein are methods for treating or preventing viral infection of a subject by administering to the subject a therapeutically effective amount of a polypeptide having at least one disintegrin domain, or a mixture of various polypeptides having at least one disintegrin domain. The polypeptide or polypeptides can be CN, VCN, or a MAP, or a fragment of CN, VCN, or a MAP. The polypeptide can also be a fusion protein comprising the amino acid sequence of either CN, VCN, or a MAP, subsequences thereof, or combinations thereof. The polypeptide can be a fusion protein with thioredoxin A or fragment thereof. The MAP can be MAP1, MAP2, MAP3, MAP6, MAP7, MAP8, MAP9, MAP10, MAP11, MAP12, MAP15, MAP17, MAP18, MAP19, MAP20, MAP21, MAP22, MAP23, MAP28, MAP29, MAP30, MAP32, or MAP33. The viral infection can be by a virus that uses a host cell integrin as a receptor for entry.

Also provided herein are methods for screening for a polypeptide comprising a disintegrin domain for antiviral activity by: (a) contacting a host cell with a candidate polypeptide and a candidate virus in either order; and (b) determining if the candidate polypeptide inhibits viral infection. The candidate polypeptide can be any polypeptide that comprises a disintegrin domain, including CN, VCN, or a MAP, or a fragment of CN, VCN, or a MAP. The polypeptide can also be a fusion protein that includes the amino acid sequence of either CN, VCN, or a MAP, subsequences thereof, or combinations thereof. The polypeptide can be a fusion protein with thioredoxin A or fragment thereof. The MAP can be MAP1, MAP2, MAP3, MAP6, MAP7, MAP8, MAP9, MAP10, MAP11, MAP12, MAP15, MAP17, MAP18, MAP19, MAP20, MAP21, MAP22, MAP23, MAP28, MAP29, MAP30, MAP32, or MAP33. The viral infection can be by a virus that uses a host cell integrin as a receptor for entry.

Also provided is a kit for one or more of: inhibiting viral entry into a host cell, treating or preventing a viral infection in a subject in need of such treatment, or screening a polypeptide having a disintegrin domain for prophylactic or therapeutic antiviral activity, the kit comprising one or more a polypeptide comprising a disintegrin domain that prevents or treats viral infection in a host cell or subject. Examples of polypeptides for use in the kit are described herein. In another aspect, the kit further comprises instructions for the intended use.

### DESCRIPTION OF THE FIGURES

FIG. 1 shows a comparison of polypeptides comprising a disintegrin domain of PIII-class snake venom metalloproteases (VAP1 and catrocollastatin) aligned with the polypeptides comprising a disintegrin domain of long-sized snake venom disintegrins (salmosin3 and bitistatin), the prototypical medium-sized snake venom disintegrin (trimestatin) as well as polypeptides including a disintegrin domain from human ADAM-derived Polypeptide (AP) in order to illustrate the rationale behind the MAP design. The structural elements generally present in polypeptides comprising a disintegrin domain of PIII-SVMPs and APs that are modified for the disintegrin domain to adopt the disintegrin fold of snake venom disintegrins are stricken through. The portion of the former spacer region that existed between the metalloprotease and disintegrin domains in the precursors of the long-sized disintegrins (e.g., bitistatin, salmosin3) and is released together with their disintegrin domains is depicted in bold.

FIG. 2 shows the alignment of selected native human AP disintegrin domains and highlights the residues that are modified (stricken through) in MAP constructs. Additionally, in two cases (the ADAM disintegrin domains 1 and 17 or AP 1 and AP 17) a native residue (bold and double-underlined) was replaced with another amino acid according to the general cysteine pattern of these artificial MAPs. The tripeptide motif located at the tip of the disintegrin loop is highlighted in a [symbol] . The amino acid residues that make the disintegrin loop in each AP are italicized.

FIG. 3 shows select MAP sequences aligned with trimestatin, a prototypical medium-size snake venom disintegrin. In the sequences shown all cysteine residues are depicted in black underline whereas the tripeptide motif at the tip of disintegrin loops in trimestatin and MAPs are in a [symbol] .

FIG. 4A-F show a listing of MAP DNA sequences that were cloned into pET32a expression vectors.

FIG. 5A-H show the amino acid sequences of TrxA-MAP constructs that were expressed in Origami® B (DE3). TrxA is thioredoxin A. The active site of TrxA and the tripeptide motif at the tip of the disintegrin loop are underlined, the TEV protease cleavage site is highlighted in a [symbol] and the linker region between TrxA and various MAP constructs is in bold black and italicized. The new residues introduced to replace the native residues in MAPs 1 and 17 are highlighted in bold double-underlined.

FIG. 6A-C show that CN blocks infection of CHO-K1 cells expressing the following gD receptors by HSV: 3-OST-3 (A), nectin-1 (B), and HVEM (C).

FIG. 7A-B show that CN blocks infection of HeLa cells (A) and Vero and 293T cells (B) by HSV.

FIG. 8A-B show that CN blocks: infection of CHO cells by HSV-1 strains F, G, and MP (A), and blocks infection HeLa cells by HSV-2 (B).

FIG. 9 (upper and lower panels) shows that CN blocks infection of RPE cells by CMV.

FIG. 10A-C show that CN, VCN and Ad15 block HSV infection of gL86 cells at MOIs of 5 PFU/cell (A), 15 PFU/cell (B), and 25 PFU/cell (C).

FIG. 11A-B show inhibition of Adenovirus infection of HeLa cells by CN.

FIG. 12A-C demonstrate that CN inhibits HSV-1 intry into CHO-K1 cells expressing gD receptors. In this experiment, Chinese hamster ovary (CHO-K1) cells expressing glycoprotein D (gD) receptors: (A) 3-OST-3, (B) herpesvirus entry mediator and (C) nectin-1 were pre-incubated with contortrostatin (CN) at indicated concentrations or mock-treated (positive control [+]) with phosphate saline buffer for 90 min at room temperature. This treatment was followed by infection with β-galactosidase-expressing recombinant virus herpes simplex virus type-1 (HSV-1; KOS) gL86 (25 pfu/cell). Uninfected cells were kept as negative control (−). After 6 h, the cells were washed, permeabilized and incubated with o-nitro-phenyl-β-o-galactopyranoside (ONPG) substrated (3.0 mg/ml) for quantitation of β-galactosidase activity expressed from the input viral genome. The enzymatic activity was measured at an optical density of 410 nm (OD 410). Error bars represent SD. a=significant difference from controls and/or treatments (P<0.05, t-test).

FIG. 13A-B show that CN inhibits HSV-1 entry into natural target cells. In this experiment (A) HeLa and (B) primary cultures of human corneal fibroblasts (CF) were used. They were either pre-treated with contortrostatin (CN) at indicated concentrations or mock-treated (positive control [+]) with phosphate saline buffer for 90 min at room temperature. β-galactosidase-expressing recombinant virus herpes simplex virus type-1 (HSV-1; KOS) gL86 (25 pfu/cell) was used to infect cells. Uninfected cells were kept as negative control (−). After 6 h, the cells were washed, permeabilized and incubated with o-nitro-phenyl-βgalactosidase activity expressed from the input viral genome. The enzymatic activity was measured at an optical density of 410 nm (OD 410). Error bars represent SD. a=significant difference from controls and/or treatments (P<0.05, t-test).

FIG. 14A-B show that HSV-1 entry blocking activity of CN is not viral-strain specific and CN inhibits HSV-1 plaque formation in cultured HeLa cells. (A) In this experiment nectin-1 Chinese hamster ovary (CHO) Ig8 cells that express β-galactosidase upon viral entry were pre-treated with 10 μM contortrostatin (CN; indicated as treated [Tr]) or mock treated (indicated as untreated [Un]) with phosphate saline buffer for 90 min at room temperature followed by infection using clinical isolates of herpes simplex virus type-1 (HSV-1; F,G, and MP at 25 pfu/cell) for 6 h. The viral entry blocking was measured by o-nitro-phenyl-β-o-galactopyranoside (ONPG) assay as previously described. Error bars represent SD. (B) Confluent monolayers of HeLa cells were infected with HSV-1 (804) strain at 0.01 PFU/cell in presence (panel i) and absence (panel ii) of CN for 2 h at 37° C. Viral replication in HeLa cells were visualized 24 h post-infection for plaques. a=significant differences from corresponding mock-treated controls (P<0.05, t-test).

FIG. 15A-B show the inhibition of HSV-1 glycoprotein-induced cell-to-cell fusion by CN and proposed model for CN-based anti-HSV-1 activity during cell entry. (A) In this experiment the naturally susceptible target human corneal fibroblasts (CF) cells expressing gD-receptors and luciferase gene were pre-incubated with 1 μM contortrostatin (CN) and mixed in 1:1 ratio with the effector Chinese hamster ovary (CHO-K1) cells expressing herpes simplex virus type-1 (HSV-1) glycoproteins (gB, gD, gH-gL) along with T7 plasmid (white bar indicated as treated [Tr]). In parallel, CN-untreated CF cells were similarly mixed with effector cells as positive control (+; black bar). The target CF cells co-cultured with effector cells devoid of HSV-1 glycoproteins were considered as negative control (−; grey bar). Membrane fusion as a means of viral spread was detected by monitoring luciferase activity. Relative luciferase units (RLUs) were determined using a Sirius luminometer (Berthoid Detection Systems, Titertek Instruments, Inc., Huntsville, Ala., USA). Error bars represent SD. (B) A cartoon illustrates the CN-mediated HSV-1 inhibition that might affect three major steps (a-c) that are involved during HSV-1 entry. (a) CN (presented by hatching) binding to host cell surface integrin (presented by α and β subunits) may affect HSV -1 glycoprotein H (gH) binding to integrins. (b) Similarly, CN-interactions with integrins may also interfere with the HSV-1 glycoprotein B (gB) binding to cell surface heparin sulfate (HSPG). (c) It is also possible that CN has higher affinity for integrins than disintegrins expressed on HSV-1 envelope glycoproteins. The interactions between cellular integrins and HSV-1 assist in viral entry, activation of downstream cell signalling molecules to enhance viral infection and activation of host immune response to facilitate disease development. a=significant differences from corresponding mock-treated controls (P<0.05, t-test).

### DETAILED DESCRIPTION OF THE INVENTION

Before the compositions and methods are described, it is to be understood that the disclosure is not limited to the particular methodologies, protocols, cell lines, assays, and reagents described, as these may vary. It is also to be understood that the terminology used herein is intended to describe particular embodiments of the present disclosure, and is in no way intended to limit the scope of the present disclosure as set forth in the appended claims.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

### Definitions

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state as well as substantially purified from an extract thereof. The term “isolated” is also used herein to refer to polypeptides, antibodies, proteins, host cells and polynucleotides that are isolated from other cellular proteins or tissues and is meant to encompass both purified and recombinant polypeptides, antibodies, proteins and polynucleotides. In other embodiments, the term “isolated” means separated from constituents, cellular and otherwise, in which the cell, tissue, polynucleotide, peptide, polypeptide, protein, antibody or fragment(s) thereof, which are normally associated in nature and can include at least 80%, or alternatively at least 85%, or alternatively at least 90%, or alternatively at least 95%, or alternatively at least 98%, purified from a cell or cellular extract. For example, an isolated polynucleotide is separated from the 3′ and 5′ contiguous nucleotides with which it is normally associated in its native or natural environment, e.g., on the chromosome. An isolated cell, for example, is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “binding” or “binds” as used herein are meant to include interactions between molecules that may be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment” as used herein, also refers to a peptide chain.

The phrases “equivalent of a peptide or polypeptide,” “biologically equivalent polypeptide” or “biologically equivalent peptide or peptide fragment” refer to a protein or a peptide fragment which is homologous to the exemplified protein or peptide fragment and which exhibit similar biological activity in vitro or in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence identity or homology. Percentage homology can be determined by sequence comparison using programs such as BLAST run under appropriate conditions. In one aspect, the program is run under default parameters.

Provided herein are methods for the inhibition of viral infection of a host cell comprising contacting the cell (in vitro or in vivo) with an effective amount of one or more polypeptides having a disintegrin domain. Also provided herein are methods for the inhibition of viral infection of a host cell comprising the administration of an effective amount of disintegrin. In one aspect of the invention, viral infection of a host cell is inhibited by the administration of an effective amount of at least one polypeptide comprising at least one disintegrin domain.

As used herein, “a polypeptide comprising a disintegrin domain” refers to one or more of a class of polypeptides that: have amino acid sequences derived from cysteine-rich proteins that are potent soluble ligands of integrins; and are involved in regulating cellular processes such as cell-cell contact, ECM adhesion, migration and invasion, cell cycle progression, cell differentiation and cell type specification that occur during the development of metazoan organisms, and cell death and apoptosis. A polypeptide comprising a disintegrin domain is meant to include: polypeptides derived from disintegrin proteins as obtained from snake venoms; polypeptides derived from disintegrin domains in mammalian ADAM proteins, including the 23 different disintegrin domains in the human family of ADAM proteins, and otherwise referred to herein as “AP” (“ADAM derived Polypeptide”); and uniquely designed polypeptides, designated as MAPs (Modified ADAM-derived Polypeptides), a “modified” form of an AP, further described herein. In some embodiments, the polypeptides comprising a disintegrin domain may be packaged in a liposomal formulation to enhance their in vivo efficacy.

In some embodiments, the polypeptide of the method is engineered to contain a bacterial thioredoxin A (TrxA) fused to the N-terminus of the polypeptide. The resultant fusion protein also comprises a unique tobacco etch virus (TEV) protease cleavage site that is engineered upstream of the polypeptide in order to facilitate its subsequent cleavage from TrxA. TEV is a highly selective protease that recognizes with very high specificity the canonical Glu-Asn-Leu-Tyr-Phe-Gln-Gly amino acid sequence, and leaves the polypeptide of the method intact.

In other embodiments, as has been shown for native CN and VCN, the polypeptide of the method may also engage integrins agonistically; thus, behaving like a soluble ECM-mimetic. As such, in various cell types, such as, but not limited to, endothelial cells and eptithelial cells, the polypeptide may elicit a cellular cascade of signaling events that rapidly lead to actin stress fibers disassembly. The polypeptide may also interfere with the assembly of a dynamic actin cytoskeleton in cells, which may result in a negative impact on the survival of these cells.

As used herein, the term “purified” in reference to polypeptides (or proteins) does not require absolute purity. Instead, it represents an indication that the polypeptide(s) of interest is(are) in an environment in which the protein is more abundant (on a mass basis) than the environment from which the protein was initially produced. Purified polypeptides may be obtained by a number of methods including, for example, chromatography, preparative electrophoresis, centrifugation, precipitation, affinity purification, etc. The degree of purity is preferably at least 10%. One or more “substantially purified” polypeptides are at least 50% of the protein content of the environment, more preferably at least 75% of the protein content of the environment, and most preferably at least 95% of the protein content of the environment. Protein content may be determined using a modification of the method of Lowry et al. [40, 41], using bovine serum albumin as a protein standard.

The tri-peptide motif RGD (Arg-Gly-Asp) is conserved in many monomeric disintegrins and is located at the tip of a flexible loop, the integrin-binding loop, which is stabilized by disulfide bonds and protruding from the main body of the peptide chain. Many disintegrins purified from snake venoms bind to the fibrinogen receptor, integrin αIIbβ3, the binding of which results in the inhibition of fibrinogen-dependent platelet aggregation. Many disintegrins also bind to integrins αvβ3 (a vitronectin receptor) and α5β1 (a fibronectin receptor) in an RGD-dependent manner.

In various embodiments of the method, the polypeptide comprising a disintegrin domain is contortrostatin (CN). As used herein, CN refers to a polypeptide comprising a disintegrin domain isolated from Agkistrodon contortrix contortrix (southern copperhead) venom [38], or an equivalent thereof. CN is produced in the snake venom gland as a multidomain precursor of 2027 bp having a 1449 bp open reading frame encoding the proprotein, metalloproteinase and disintegrin domains. The precursor is proteolytically processed, possibly autocatalytically, to generate mature CN. The full length CN preprotein is encoded by the nucleotide sequence 85-1536 of the full length mRNA (GeneBank AF212305), whereas the disintegrin domain of CN represents 1339-1533 of the mRNA. The CN disintegrin domain, which contains 65 amino acids, is shown below with the RGD sequence underlined.

DAPANPCCDAATCKLTTGSQCADGLCCDQCKFMKEGTVCRRARGDDLD
DYCNGISAGCPRNPFHA

Mature CN polypeptides form homodimers linked together by two disulfide bridges. In various embodiments, those disulfide bridges form between the first cysteine residue (position 1345) of one CN subunit, and the third cysteine residue (position 1351) of the other CN subunit, and vice versa to form two interchain disulfide bridges in an antiparallel orientation (the first cysteine residue of one subunit pairs with the third one of the other subunit and vice versa). The CN homodimer has a molecular mass (Mr) of 13,505 kDa, and and the reduced monomer has Mr of 6,750 kDa [38].

In other embodiments of the method, the polypeptide comprising a disintegrin domain is vicrostatin (VCN). VCN is a chimeric disintegrin generated recombinantly by grafting the C-terminal tail of viperid snake venom disintegrin echistatin to the sequence of crotalid disintegrin CN or an equivalent thereof. In various embodiments, VCN may be produced as an active polypeptide in Origami® B E. coli. VCN retains the binding profile of CN yet it engages integrins in a unique manner.

The VCN disintegrin domain is disclosed in U.S. Pat. No. 7,754,850, issued Jul. 13, 2010, and is shown below with the RGD sequence underlined.

DAPANPCCDAATCKLTTGSQCADGLCCDQCKFMKEGTVCRRARGDDLD
DYCNGISAGCPRNPHKGPAT

When TrxA-VCN fusion proteins are cleaved by TEV protease, the VCN sequence may include an N-terminal Gly that was part of the TEV protease cleavage site. Additional amino acids at the N-terminus of VCN are not known to impact the activity of the molecule.

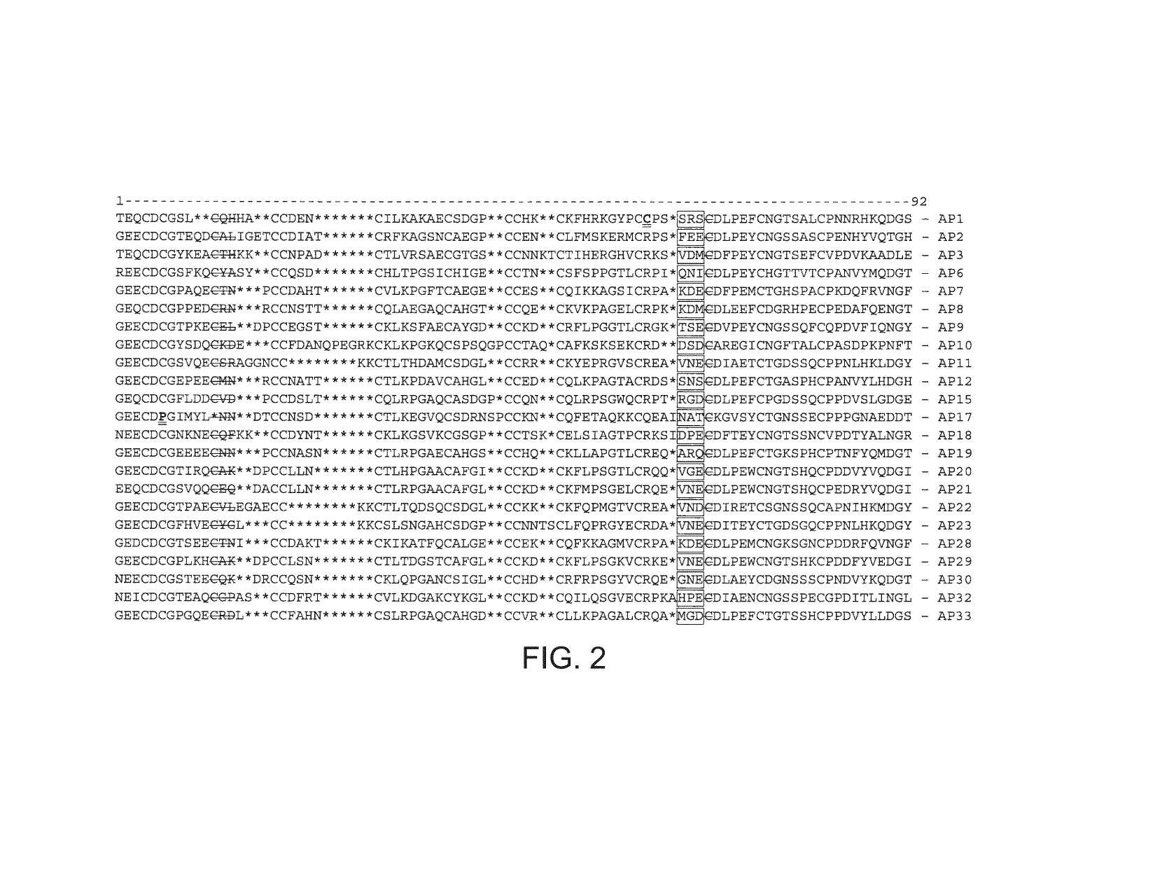

In some embodiments, the polypeptide may belong to a class of uniquely designed polypeptides that are termed MAPs (Modified ADAM-derived Polypeptides). As used herein, MAPs refer to a sequence modified form of the native disintegrin domain of an ADAM protein or an equivalent thereof. As used herein, a “disintegrin domain of an ADAM protein” which may be referred to herein as “AP” (“ADAM derived Polypeptide”) is a disintegrin domain of the ADAM which has been separated from its metalloprotease and cysteine-rich domains and from any interdomain segments. Examples of APs are shown in FIG. 2. The AP is a fragment of ADAM. In some embodiments of the invention, the AP may comprise conservative amino acid substitutions, deletions, or fragments thereof. In some embodiments, the AP amino acid sequence extends from the third amino acid residue in the N-terminal direction from the ADAM CDC motif to the tenth amino acid residue in the C-terminal direction from the twelfth cysteine residue of the CDC motif. In other embodiments, the AP amino acid sequence contains the aforementioned AP sequence plus any of the amino acid residues in the N-terminal direction up to, but not including the next cysteine residue in the N-terminal direction from the CDC motif, and any of the amino acid residues in the C-terminal direction from the tenth amino acid residue described above up to, but not including the next cysteine residue in the C-terminal direction. See e.g. FIG. 2. There are two exceptions: (1) the C-terminal end of AP1 is defined as the position 10 amino acid residues C-terminal from the 13th cysteine residue from the CDC motif up to but not including the next cysteine C-terminal to said 13^{th} cysteine residue, and (2) ADAM 17 has a CDP motif rather than a CDC motif from which the ends of the corresponding AP (AP 17) are defined.

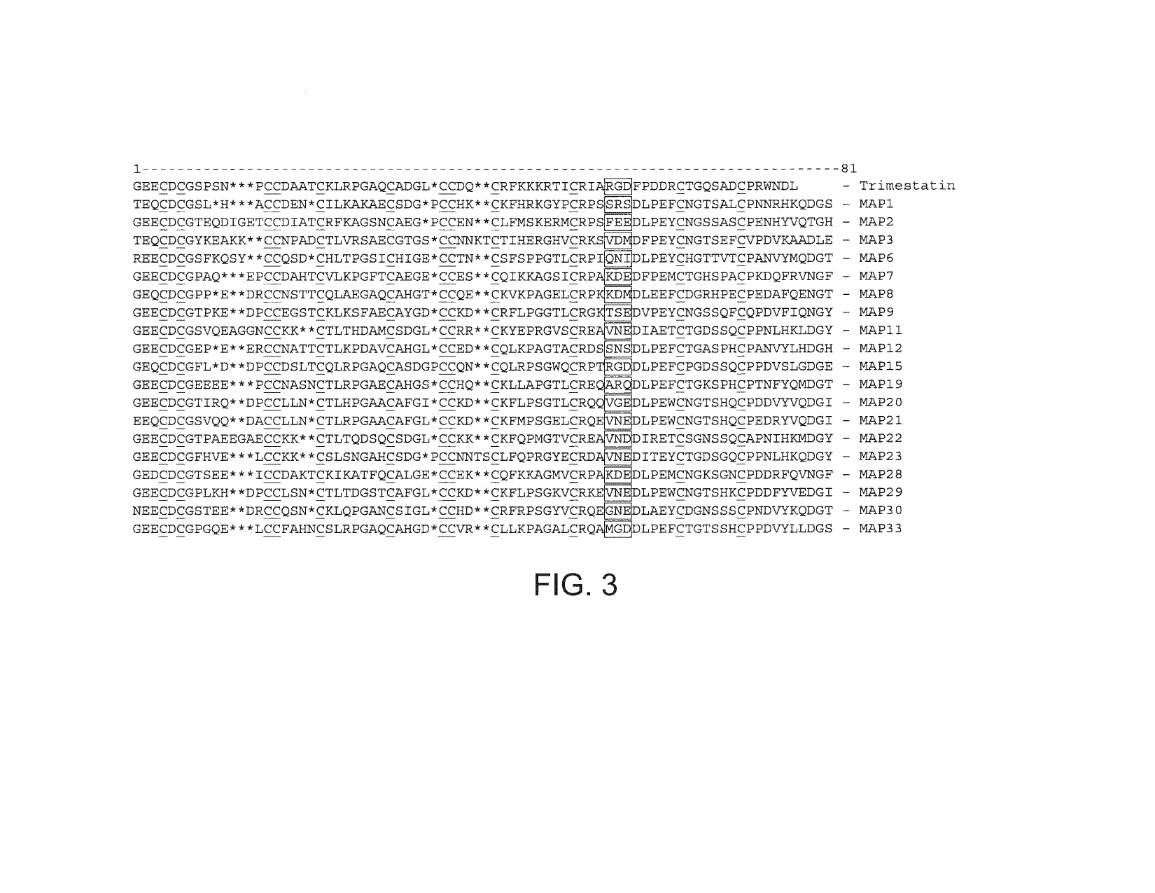

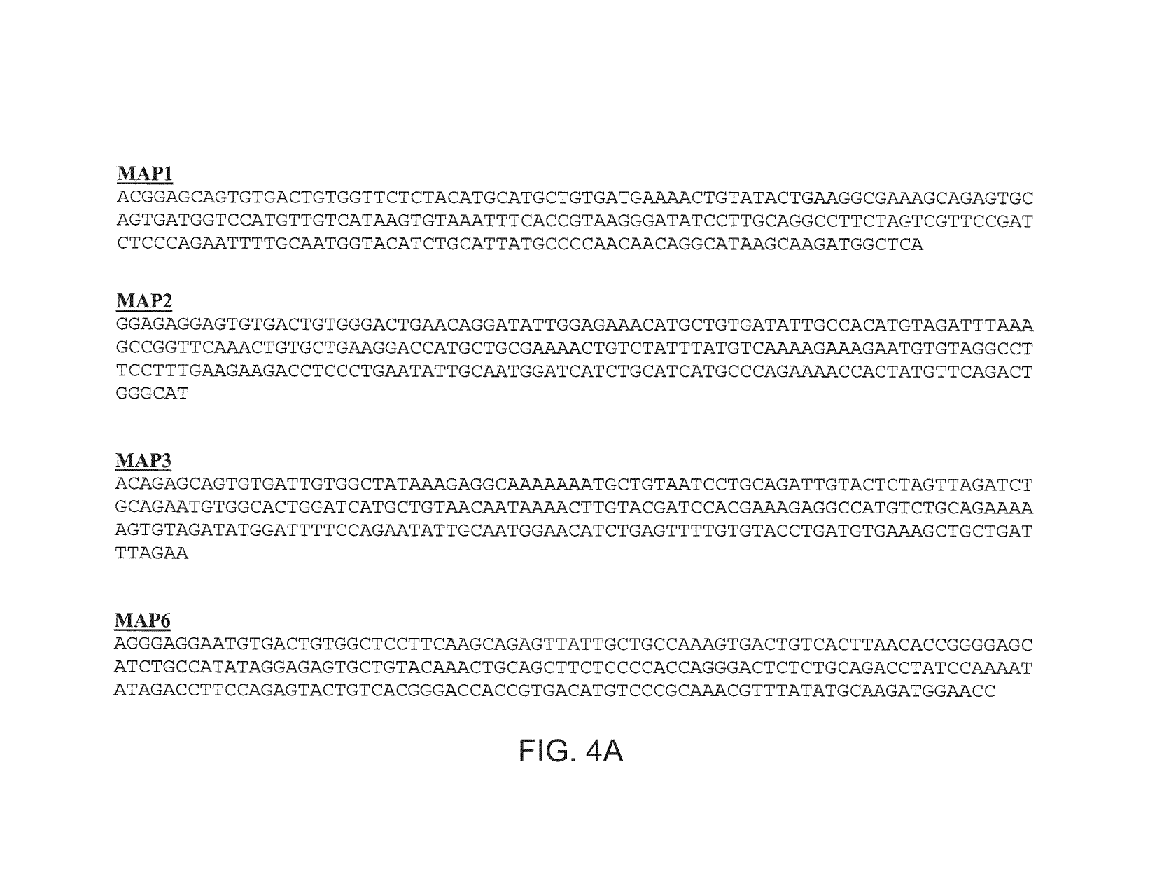

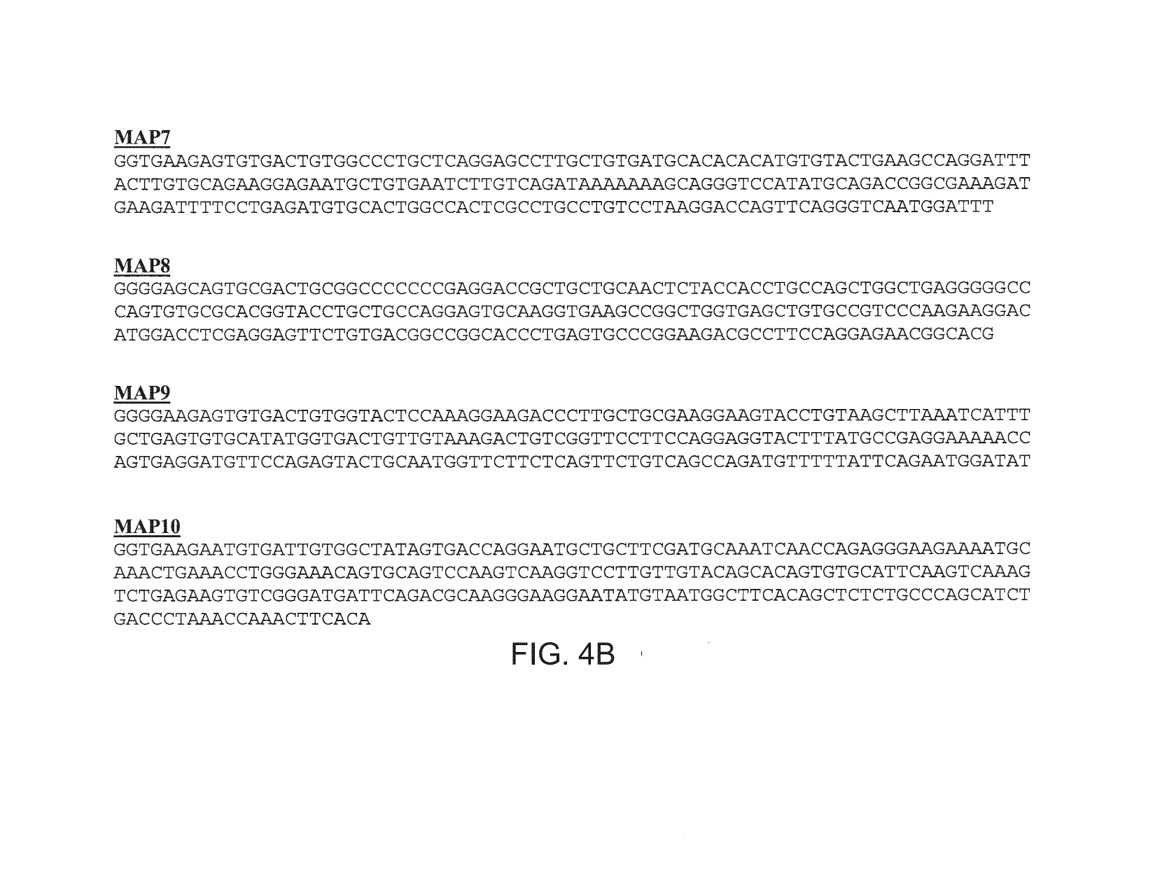

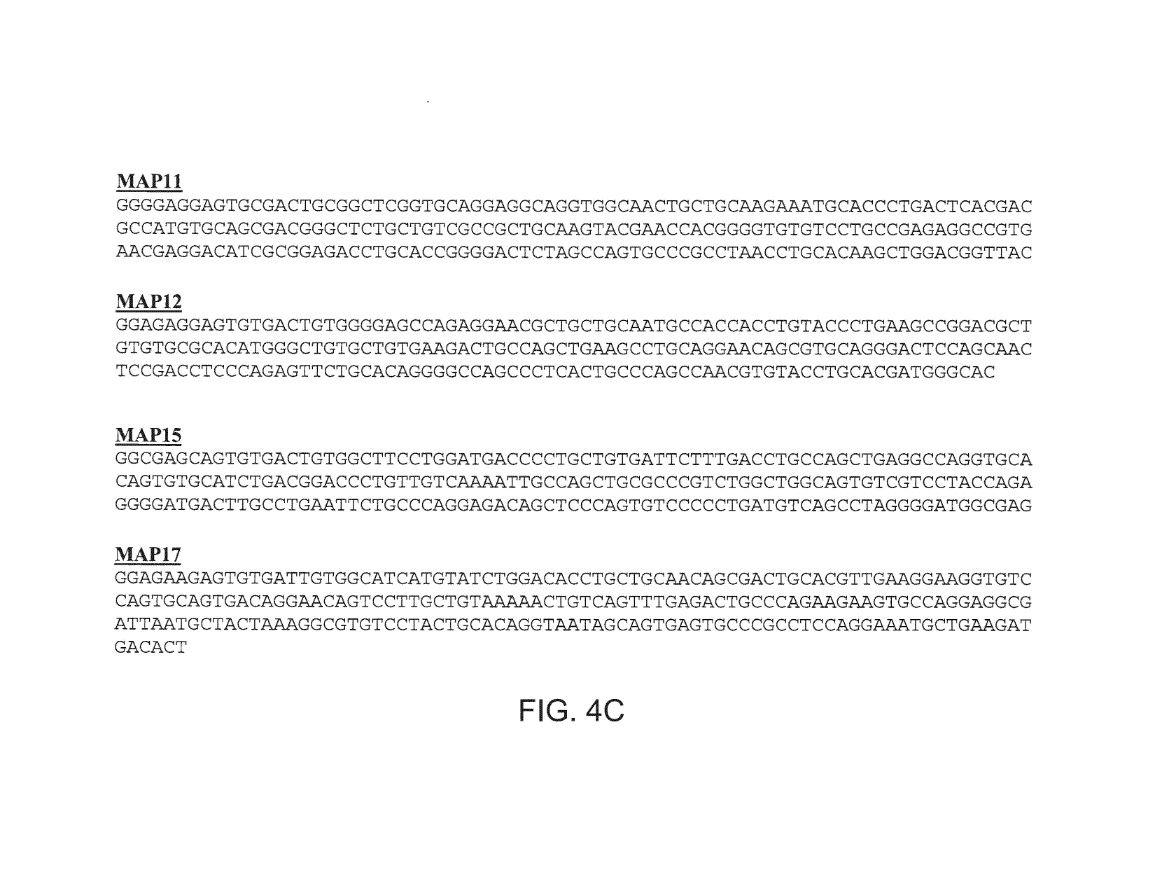

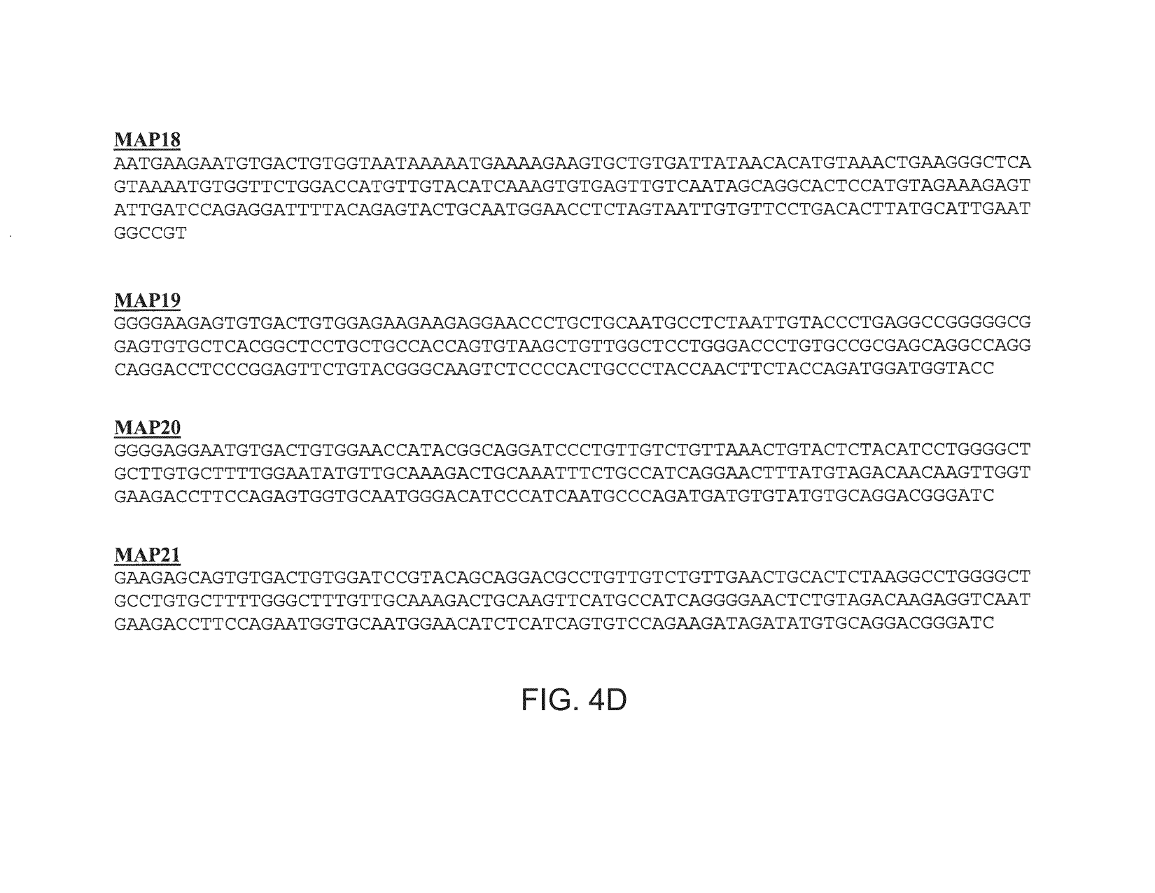

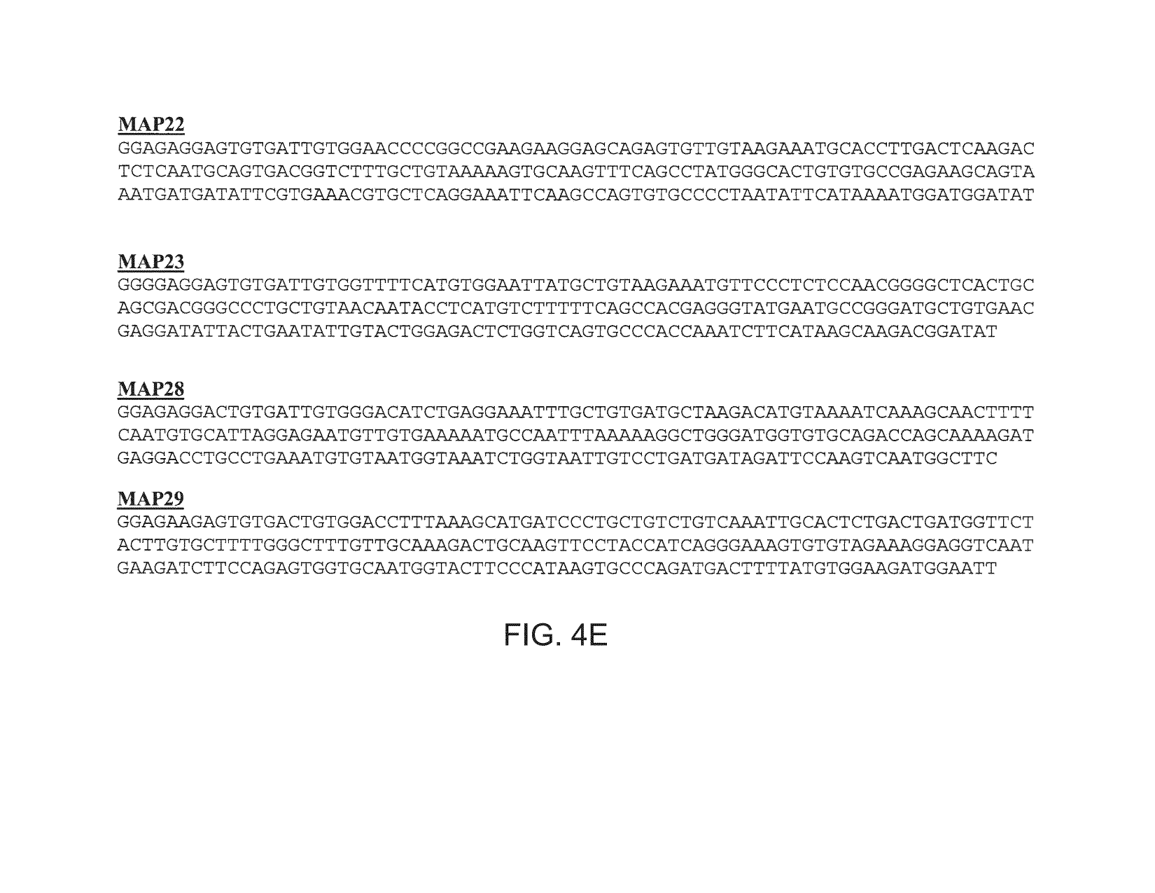

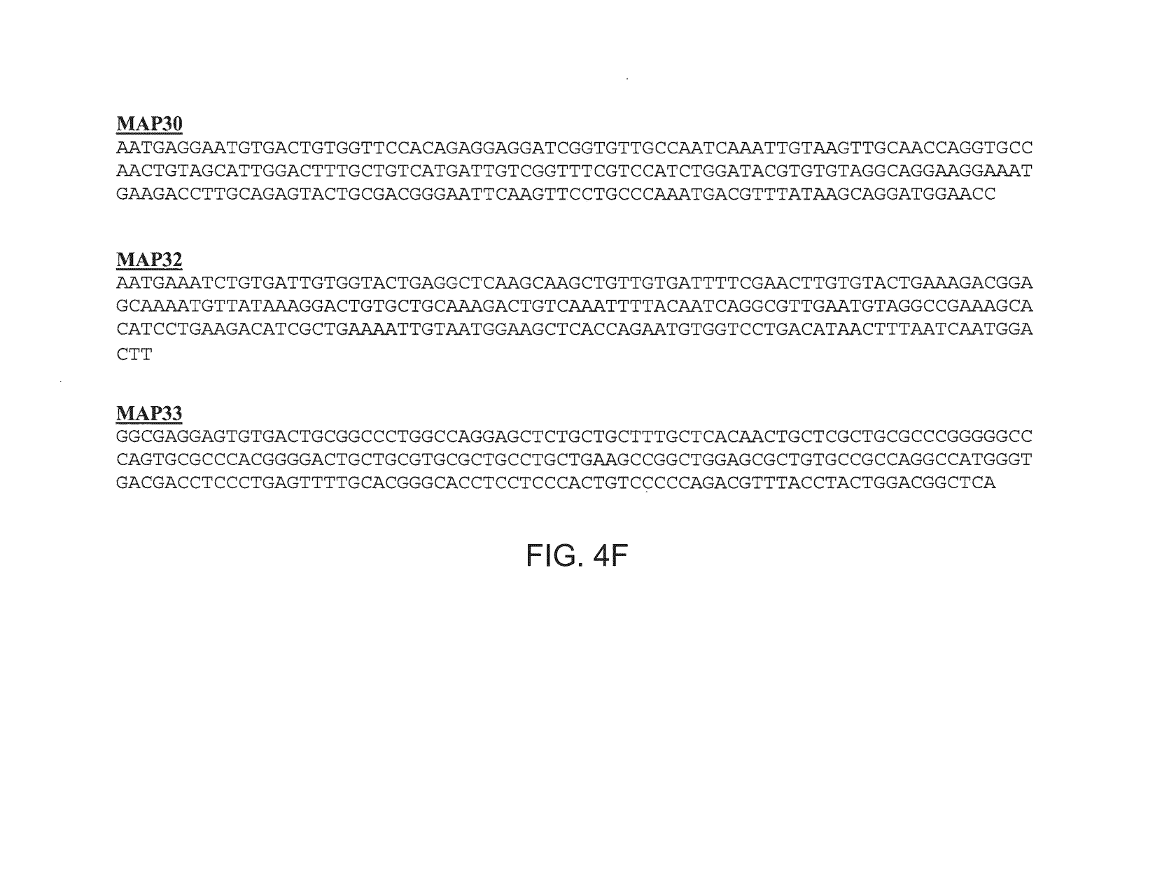

As stated above, a “MAP” is a “modified” form of an AP. The modifications may involve an alteration(s) in the sequence of the AP to achieve the beneficial properties described herein. MAPs, therefore, have amino acid sequences which are modified relative to the sequence normally present in the AP and the corresponding sequence of the ADAM parent. As used herein, “modified” means that the amino acid is deleted, substituted or chemically treated and, in an embodiment, the modification results in disruption of interdomain disulfide linkage. Exemplary MAPs are shown in FIG. 3. The MAP sequences are shown aligned with trimestatin, a prototypical medium-size snake venom disintegrin. All MAP constructs are modeled after medium-size snake venom disintegrins and had their sequences modified to fold similarly to these native snake venom molecules. The MAPs, with the exception of MAP17, are constructed such that the first cysteine in the C-terminal direction from the CDC motif and the next two amino acid residues in the C-terminal direction, as well as the cysteine residue in the C-terminal direction to the AP tripeptide motif are deleted.

Alternatively, the Cys residues can be substituted with alternate amino acids or the Cys amino acid residues can be chemically modified such as to prevent disulfide bond formation. The amino acid substitutions can be conservative, e.g. the first Cys C-terminal to the CDC motif of the AP can be substituted with a serine residue, the amino acid residues C-terminal to said cysteine can be substituted with a charged amino acid, or the Cys C-terminal to the tripeptide motif can be substituted with a charged amino acid. Such mutational approaches and chemical treatments are well known in the art. With regard to chemical treatments, an example is the use alkylating agents to react with cysteine residues to prevent formation of disulfide bonds. Except for MAP10, 17, 18 and 32, MAPs display an 11 amino acid disintegrin loop, similar to the native loop of snake venom disintegrins. MAP 10 displays a 10 amino acid integrin loop and MAP17, MAP18, and MAP32 display a 12 amino acid disintegrin loop.

MAPs can be expressed and further purified as stand alone biologically active molecules in a bacterial system that supports both the generation of active soluble disulfide-rich polypeptides and high expression yields for these products. While not wishing to be held by theory, the MAPs were designed from the native APs so that they could adopt a snake venom disintegrin fold rather than their native ADAM conformations. The MAPs can be expressed with high yields in the Origami B (DE3) E. coli strain and further purified as stable and active free polypeptides that can interact with a class of mammalian cell surface receptors, the integrins, in a manner that is similar to that of native snake venom disintegrins. The MAPs also retain some of the signaling properties that are characteristic of the APs or disintegrin domain activities from the ADAM parent from which the MAP was derived form. For instance, retained characteristics may include signaling attributes related to the putative ability of the ADAM disintegrin domains to engage integrin receptors by utilizing amino acid residues located outside the classical disintegrin loop. Cellular functions of ADAMs are well known [42-47].

Although not wishing to be bound by theory, it is believed that the PII-class SVMPs that give rise to the prototypical medium-sized snake venom disintegrins (e.g., Trimestatin, Kistrin, Flavoridin etc) fail to form a critical disulfide bridge between the upstream spacer region and the disintegrin domain and thus the proteolytic attack happens in the residues located just N-terminally of where the disintegrin domain starts, the consequence of this being that the released medium-sized disintegrins are complete disintegrin domains containing no portion of the upstream spacer region. In contrast, it is believed that the PII-class SVMPs that give rise to the long-sized snake venom disintegrins (e.g., bitistatin, salmosin3 etc) fail to form a critical disulfide bridge between the metalloprotease domain and the downstream spacer region and consequently a proteolytic attack does happen more upstream in the spacer region with the release of a longer disintegrin.

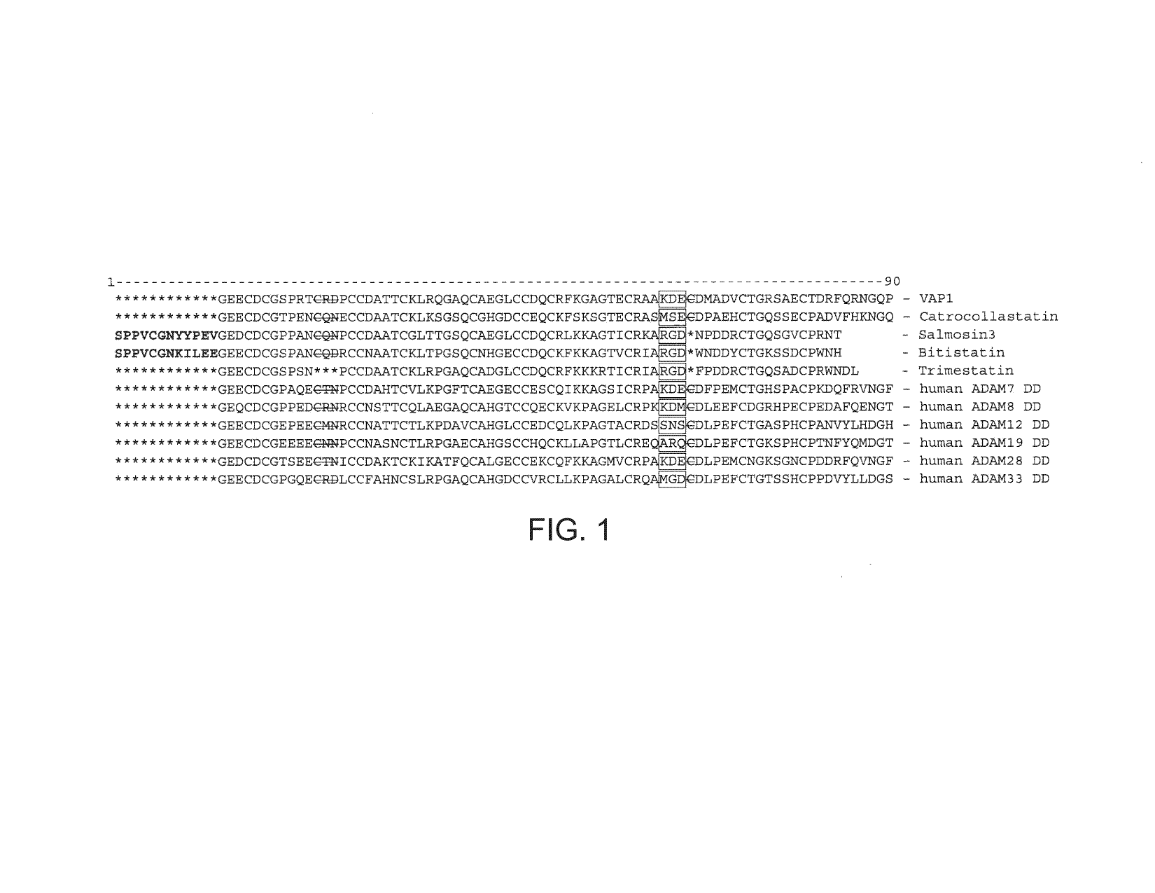

Because in this case the proteolytic event is believed to happen upstream of a disulfide bridge that still forms between the spacer and the disintegrin domain, the long-sized snake venom disintegrins are released with a portion of the spacer region attached N-terminally to the freed disintegrin domain (see the sequence alignment of various disintegrin and disintegrin domains in FIGS. 1-3). Moreover, it is also believed that when the PII-SVMPs contain even more mutations and/or deletions, the disulfide bridges fail to form in the same spacer region but also in the N-terminal part of the disintegrin domain and even shorter variants of snake venom disintegrins are released (e.g., either partially truncated disintegrins domains that dimerize like contortrostatin or, more rarely, extremely truncated polypeptides like echistatin or eristostatin). It is further believed that in almost all cases, the free disintegrin domains display a conserved 11-amino acid disintegrin loop in the C-terminal half of their molecule, which is the hallmark of snake venom disintegrins.

The 23 different ADAM gene sequences that have been identified in the human genome (3 of them being pseudogenes that are not normally translated into a protein product) have been modified as described herein such that the modified ADAM proteins adopt the snake venom disintegrin fold.

Several ADAM transcripts have a number of isoforms. Nonetheless, among the isoforms of the known ADAMs, the coding sequences of the disintegrin domains are conserved; therefore, there are only 23 different disintegrin domains in the human family of ADAM proteins. When produced recombinantly, the MAPs of the invention can interact in a high affinity manner with a defined integrin set. This property makes these mutant polypeptides broad spectrum integrin ligands for clinical and therapeutic use.

Similar to the other human ADAM members, the non-functional transcripts do contain complete disintegrin sequences that, if artificially translated in a recombinant system, can generate active polypeptides with novel biological functions. The disintegrin domains of human ADAMs have between 76 to 86 amino acids (the disintegrin domain of ADAM1 is the shortest, whereas that of ADAM10 is the longest), and with 2 exceptions (ADAMs 1 and 17), they all contain the 14 canonical cysteine residues of the original ADAM scaffold (see the aligned sequences of human ADAMs below). Unlike the snake venom disintegrins, that naturally evolved to function as platelet aggregation inhibitors, most which contain an RGD tripeptide motif at the tip of their disintegrin loop, the disintegrin loops of ADAMs display much different tripeptide motifs at their tips and therefore are expected to engage a broader range of integrins and in a different manner than their snake venom counterparts. In fact, each of the APs is believed to bind to a defined set of integrin receptors thus signaling in a unique manner (see FIG. 1 for the sequence alignment of ADAM and snake venom disintegrins illustrating the differences in the disintegrin loops).

The disintegrin domain of human ADAM15 contains a RGD tripeptide motif in its disintegrin loop which supports the hypothesis that human ADAM15 plays important regulatory roles in the cardiovascular system.

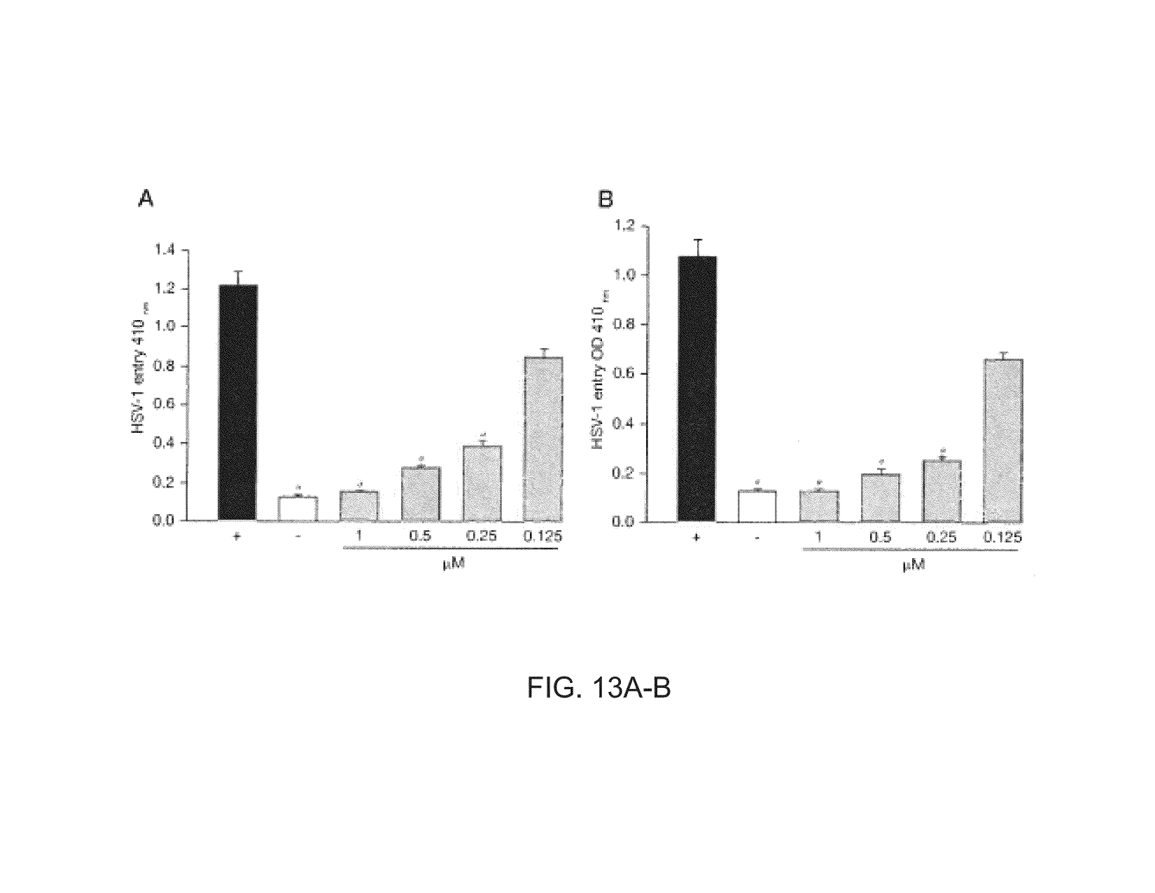

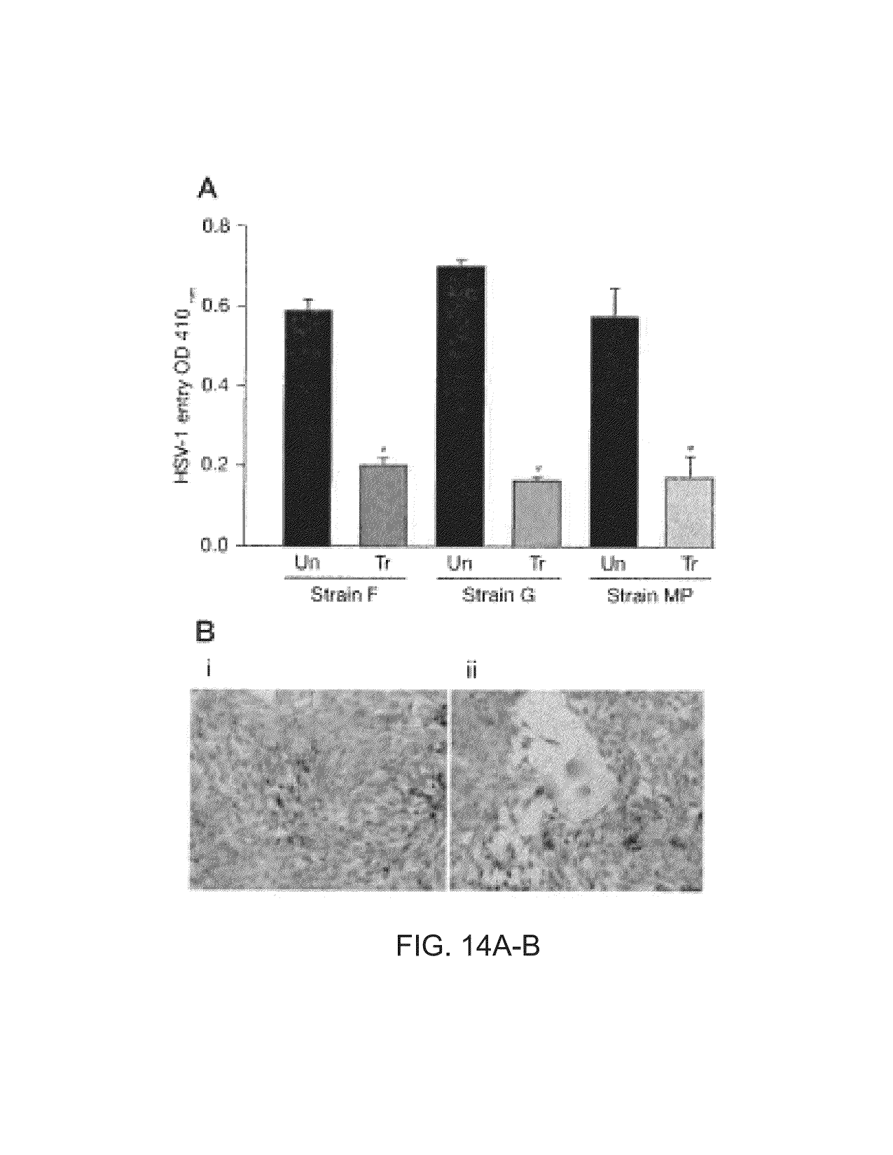

MAPs for each AP portion of all 23 known human ADAM members were generated. The human ADAM disintegrin domain sequences were modified according to the rationale presented above, which includes removing the residues (among which include 2 cysteine residues) in the ADAM disintegrin domain that normally participate in interdomain-disintegrin domain disulfide bridge formation in the native ADAM proteins. Not wishing to be held by theory, the apparent function of these disulfide bridges is to keep the disintegrin loops in ADAMs tightly packed and unavailable to integrin receptors. By removing the residues that participate in the formation of these disulfide bridges, these MAPs acquire the mobility of the canonical 11-amino acid loop and the disintegrin-fold characteristic of snake venom disintegrins. Among the 23 members of the human ADAMs, 6 members perfectly fit the above-mentioned scheme (ADAMs 7, 8, 12, 19, 28 and 33) when aligned with long- and medium-sized snake venom disintegrins as well as with PIII-class SVMPs (see FIG. 1 for an alignment of snake venom disintegrins and human ADAM disintegrin domains). Nonetheless, by introducing these modifications, with the exception of 4 ADAMs (10, 17, 18 and 32), all human ADAM members were converted to MAPs that display a 11-amino acid disintegrin loop. Regarding the 4 exceptions, 3 (ADAMs 17, 18 and 32) were converted to MAPs displaying a slightly longer, 12-amino acid loop, while 1 member (ADAM 10) was converted to a MAP carrying a slightly shorter 10-amino acid disintegrin loop (see AP10 in FIG. 14 for a sequence alignment). Moreover, in the case of 2 APs (ADAMs 1 and 17), one additional native residue in each sequence was replaced with either an arginine residue (to generate MAP1) or a cysteine residue (to generate MAP17) to restore the cysteine pattern characteristic of disintegrin domains (see FIG. 14 for sequence alignment).

As used herein, “interdomain regions” or “spacer regions” means the polypeptide portion of an ADAM between the metalloprotease and disintegrin domain (the “MD interdomain region”) and between the disintegrin domain and the cysteine-rich domain (the “DC interdomain region”), respectively, wherein the MD interdomain region starts at least 10 amino acid residues N-terminal to the AP and the DC interdomain region starts at least 10 amino acid residues C-terminal to the AP. Each interdomain is 5 to 15 amino acids in length.

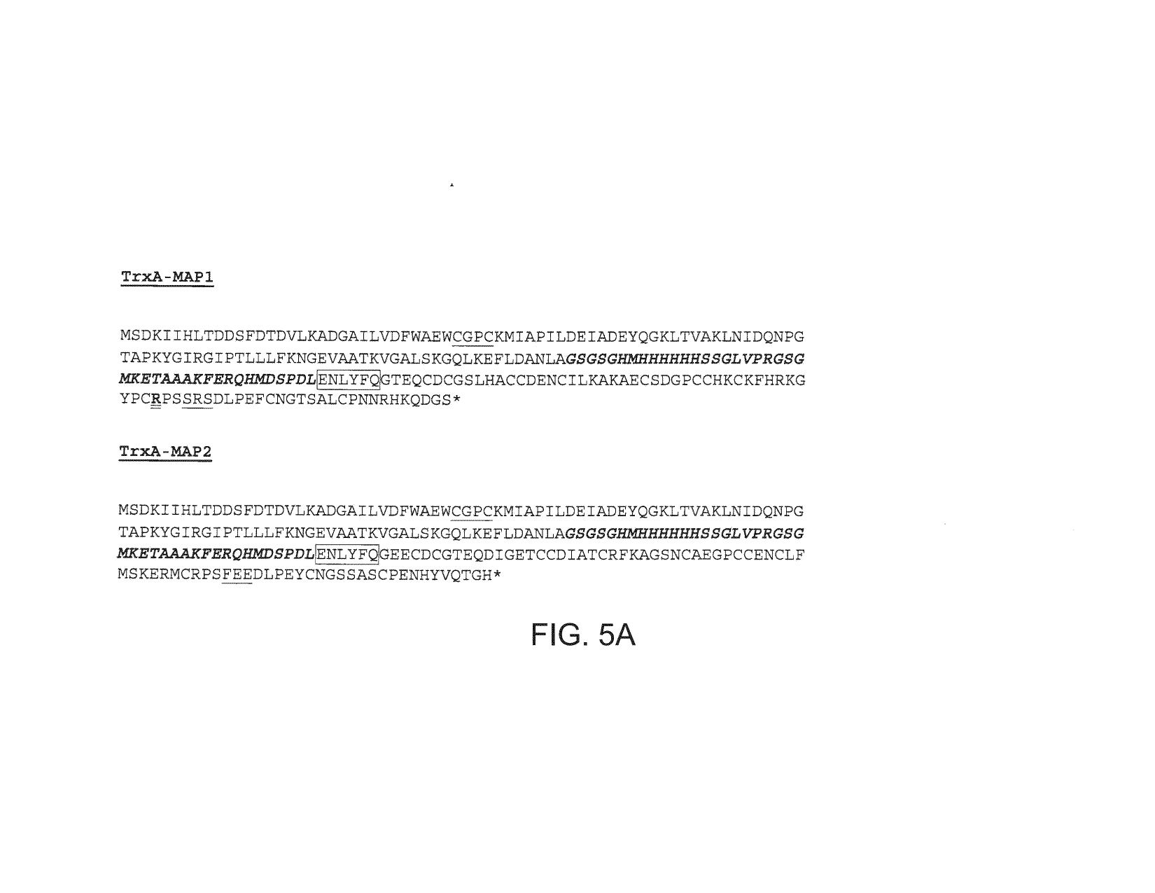

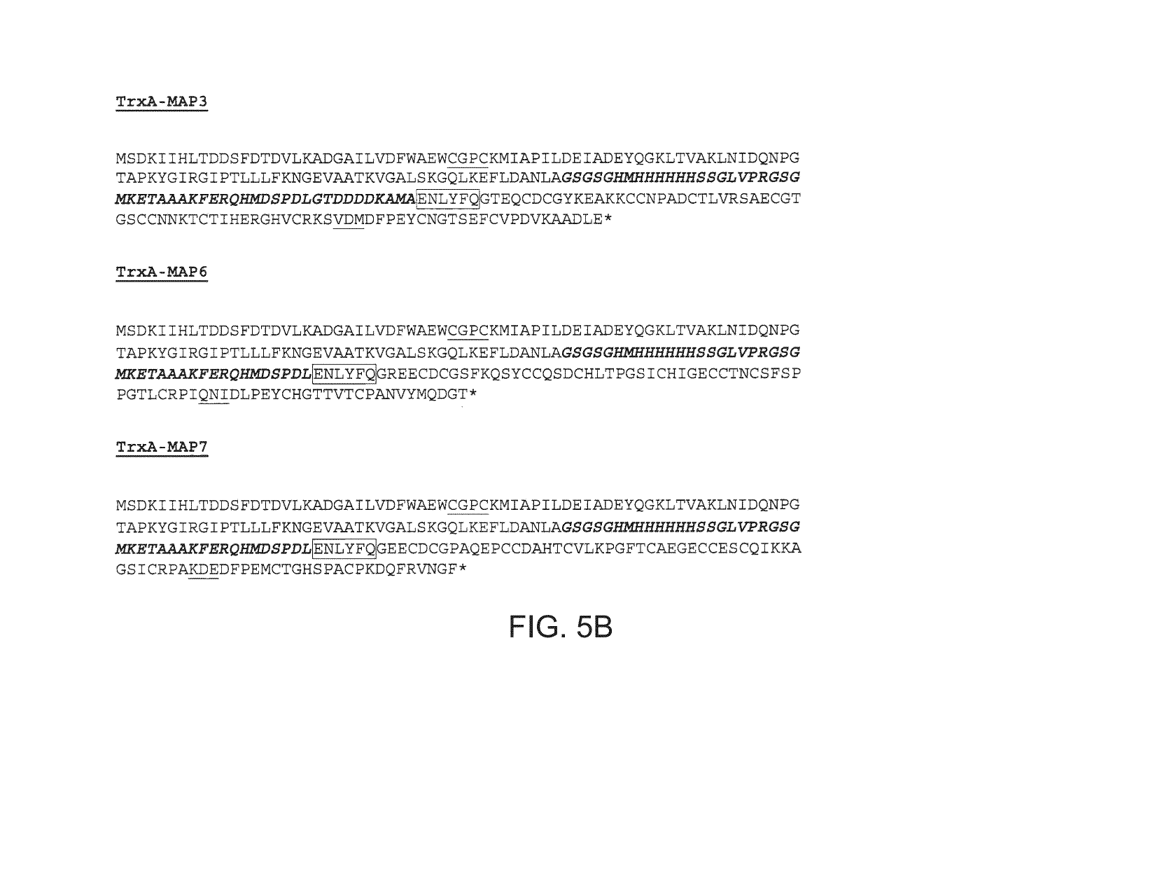

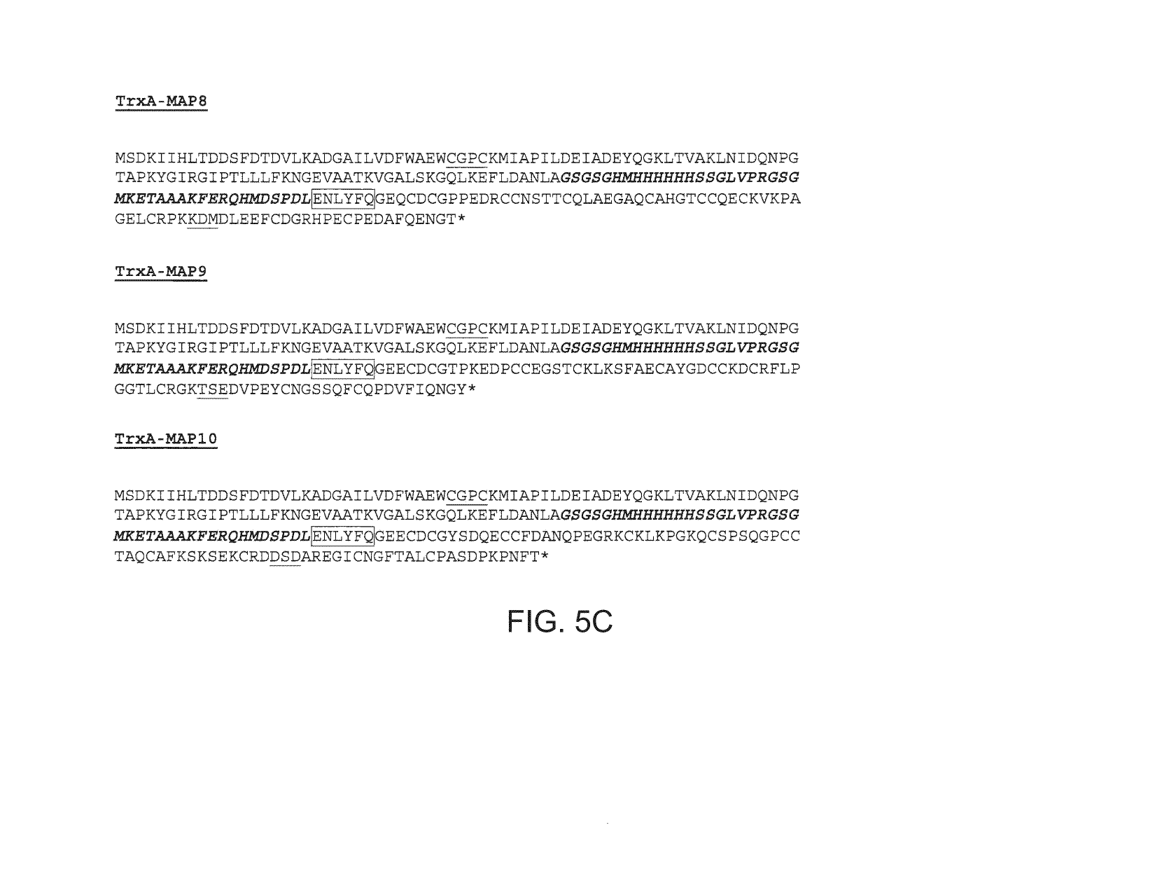

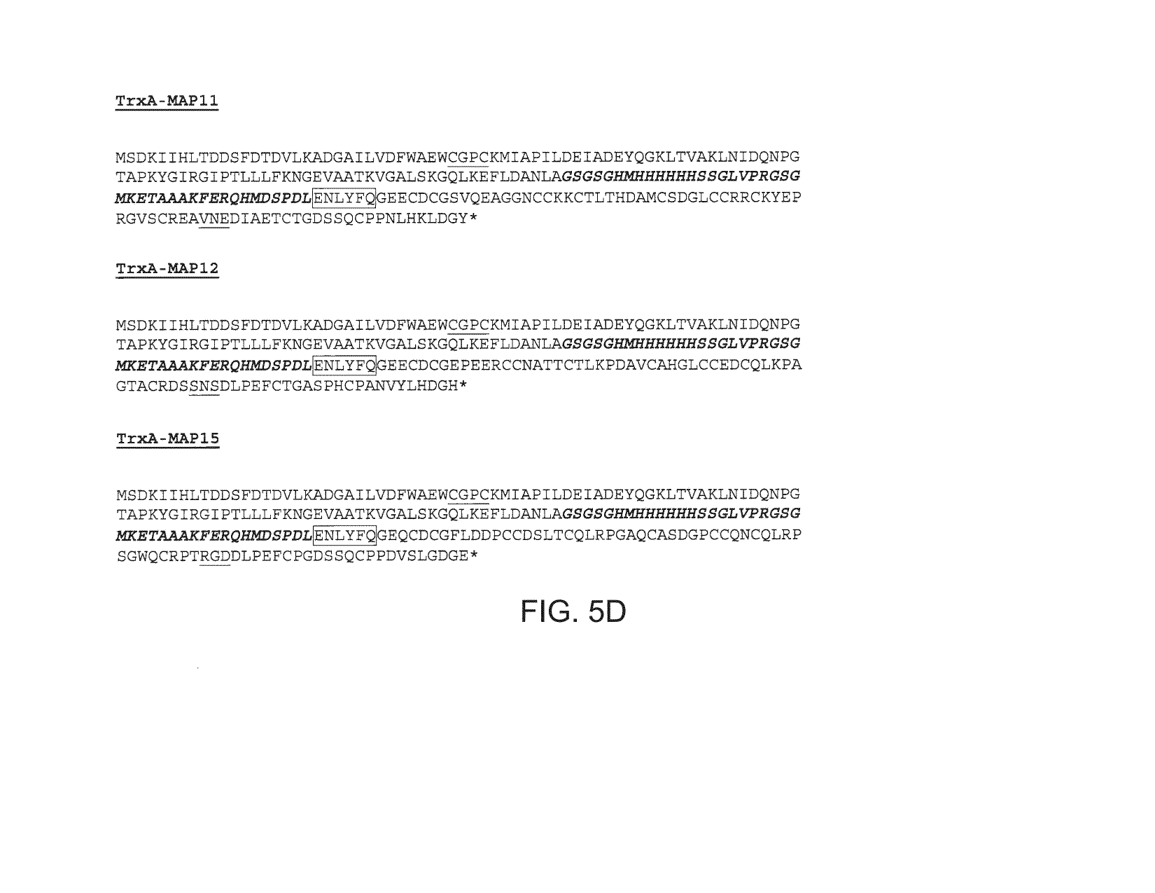

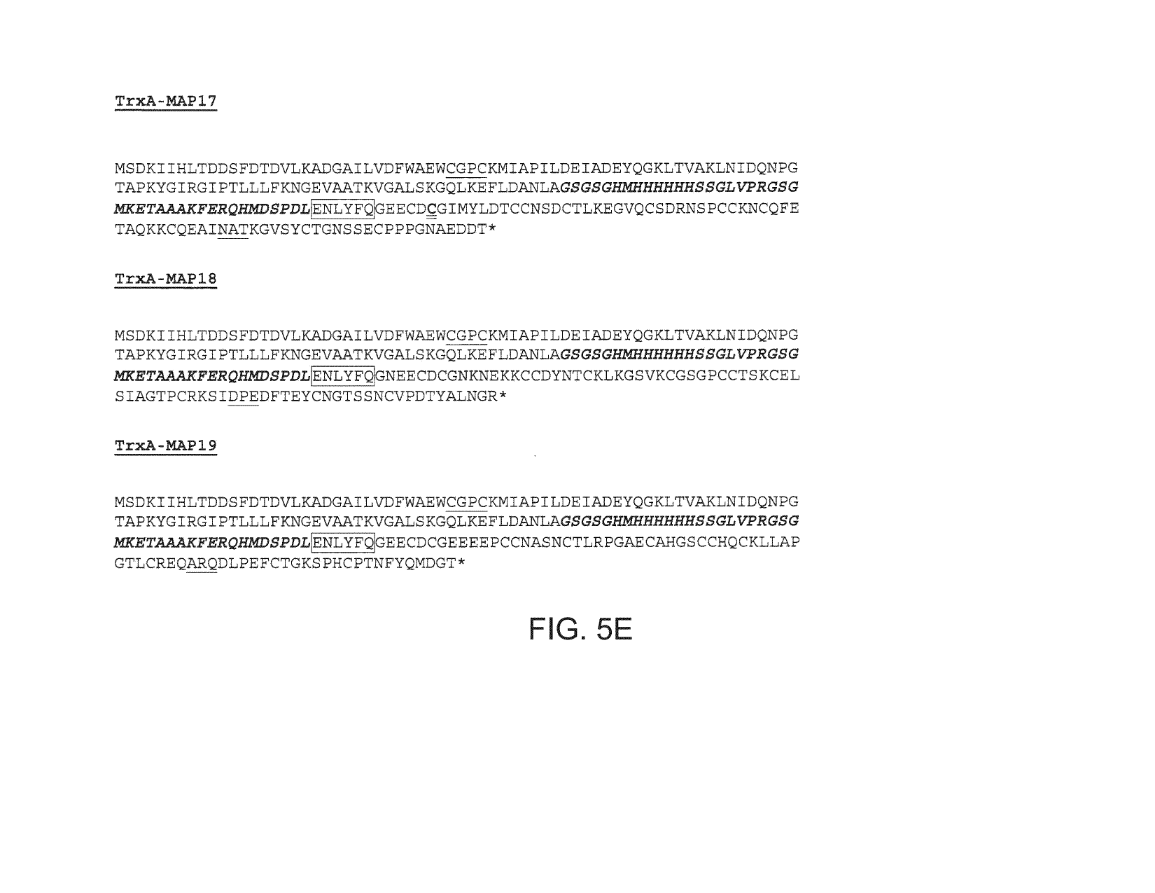

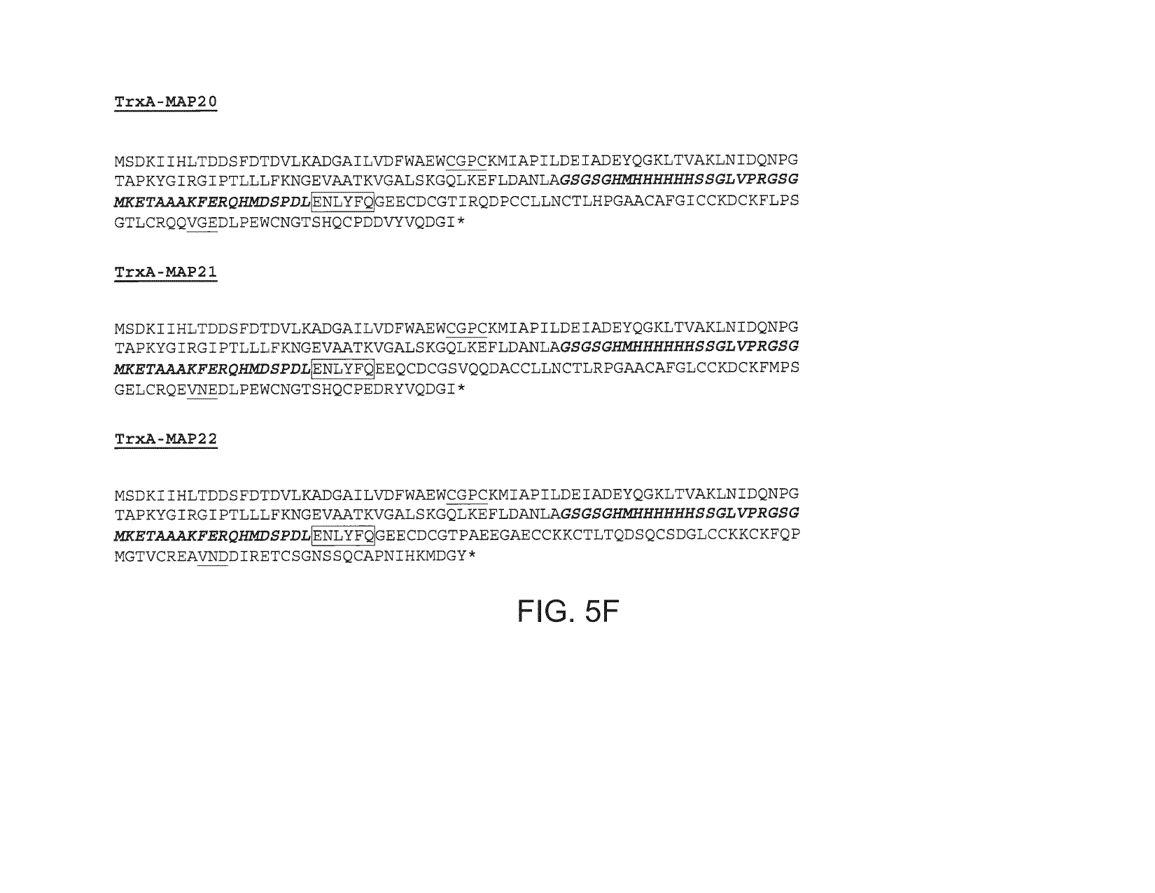

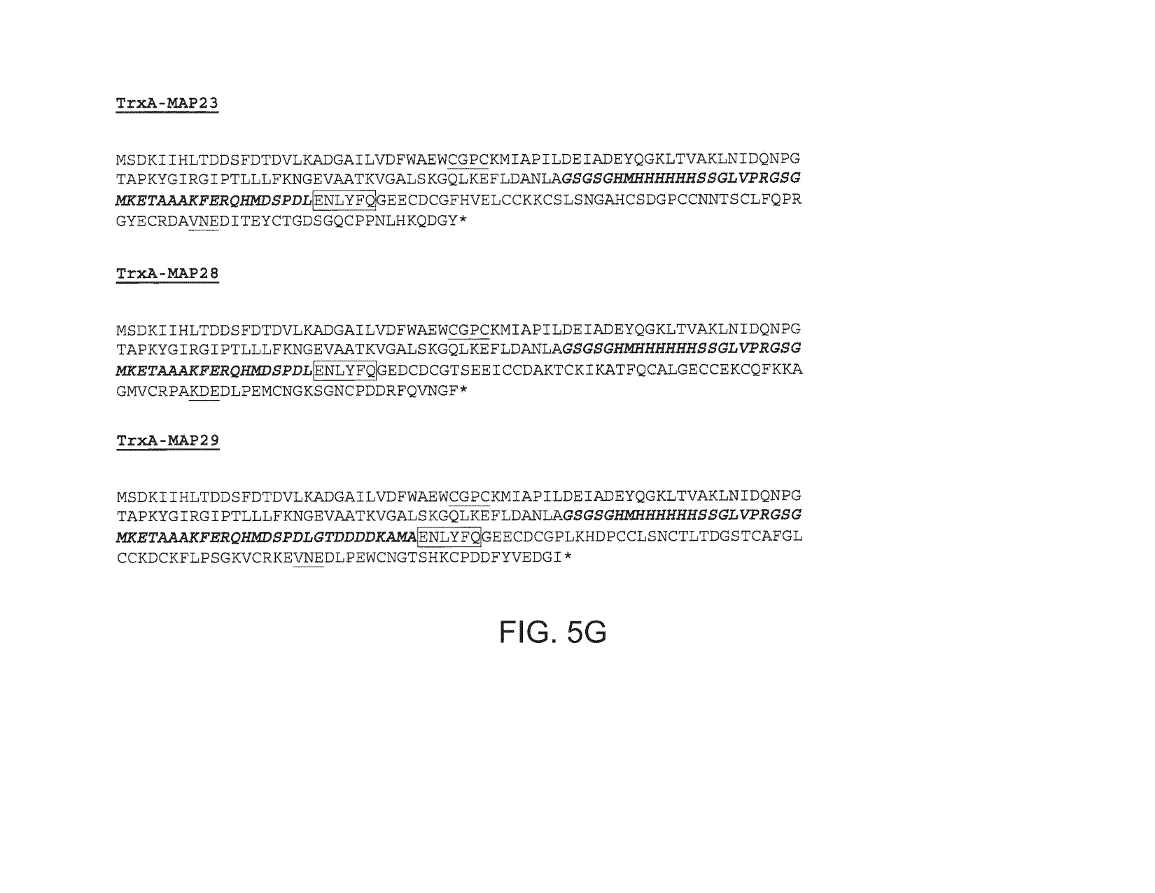

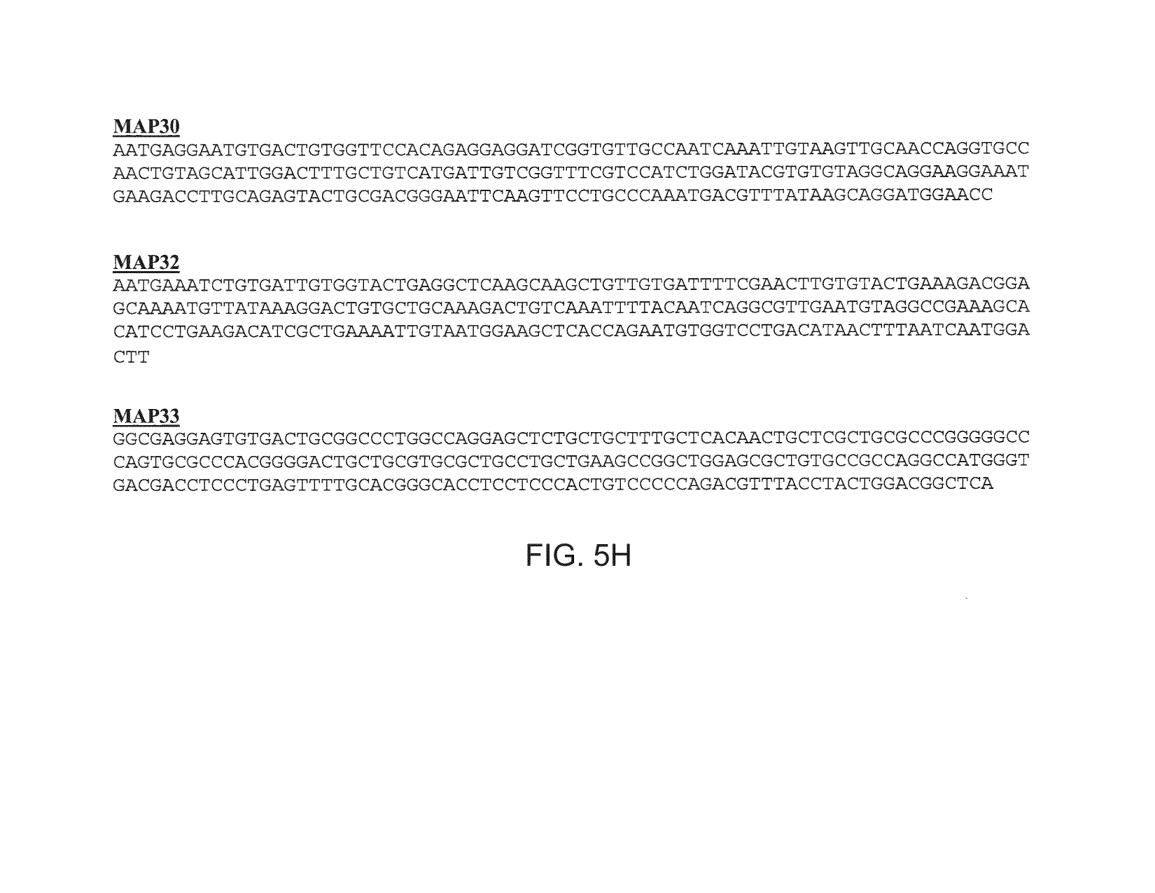

The DNA sequences of all 23 MAPs were de novo synthesized and cloned into the pET32a expression vector [48] downstream of bacterial thioredoxin A (TrxA). The amino acid sequences of TrxA-MAP constructs that were expressed in Origami B (DE3) are shown in FIG. 5. The MAPs were produced in the Origami® B (DE3) bacterial strain as described in PCT Patent Application No. PCT/US09/64256, filed Nov. 12, 2009, and titled “Method of expressing proteins with disulfide bridges with enhanced yields and activity.” This application describes an improvement upon the expression system disclosed in U.S. Publication No. 20060246541 which includes, as an embodiment, expression of a chimeric snake venom disintegrin vicrostatin (VCN) in the Origami B (DE3)/pET32a system. The improved method was used to generate increased amounts of correctly-folded active MAPs. This is achieved by growing the Origami B cells in a less selective environment and thus allowing for the generation and expansion of VCN-transformants that display a more optimal redox environment during the induction of the heterologous recombinant protein production. Unlike other E. coli strains, the Origami B is unique in that, by carrying mutations in two key genes, thioredoxin reductase (trxB) and glutathione reductase (gor), that are critically involved in the control of the two major oxido-reductive pathways in E. coli, this bacterium cytoplasmic microenvironment is artificially shifted to a more oxidative redox state, which is the catalyst state for disulfide bridge formation in proteins [49, 50].

The Origami B strain has growth rates and biomass yields similar to those obtained with wild-type E. coli strains, which makes it an attractive and scalable production alternative for difficult-to-express recombinant proteins like VCN. This strain is also derived from a lacZY mutant of BL21. The lacY1 deletion mutants of BL21 (the original Tuner strains) enable adjustable levels of protein expression by all cells in culture. The lac permease (lacY1) mutation allows uniform entry of IPTG (a lactose derivative) into all cells in the population, which produces a controlled, more homogenous induction. By adjusting the concentration of IPTG, the expression of target proteins can be optimized and theoretically maximal levels could be achieved at significantly lower levels of IPTG. Thus the Origami B combines the desirable characteristics of BL21 (deficient in ompT and lon proteases), Tuner (lacZY mutant) and Origami (trxB/gor mutant) hosts in one strain. As mentioned above, the mutations in both the thioredoxin reductase (trxB) and glutathione reductase (gor) greatly promote disulfide bond formation in the cytoplasm [50].

Although the Origami® B strain offers a clear advantage over E. coli strains with reducing cytoplasmic environments like BL21, the mere usage of the Origami® B strain and the pET32a expression vector does not automatically guarantee the generation of a soluble and/or active product. The generation of disulfide-rich polypeptides in Origami B appears to be sequence dependent. For example, some MAPs (e.g. MAP9 and MAP15) can be expressed in Origami® B with significantly higher expression yields compared to their corresponding AP versions of human ADAMs 9 and 15, despite the fact that the same system and production technique were employed. Consequently, the modification of APs into MAPs can result in polypeptides having a disintegrin domain with greater expression yield in Origami B cells.

Furthermore, after purifying expressed disintegrin domains (APs) of ADAM 9 and 15, in a process that involves TEV protease treatment and RP-HPLC purification, the collected free polypeptides appeared to be unstable and to precipitate out of solution after reconstitution from lyophilized powder. In contrast, the corresponding MAP polypeptides, generated by employing the same purification steps, appear to be much more soluble and stable when reconstituted in water after lyophilization.

Polypeptides comprising a disintegrin domain are prepared as described herein so as to be isolated or purified. As used herein, the term “purified” (or isolated) in reference to polypeptides comprising a disintegrin domain does not require absolute purity. Instead, it represents an indication that a preparation of polypeptides comprising a disintegrin domain are preferably greater than 50% pure, more preferably at least 75% pure, and most preferably at least 95% pure, at least 99% pure and most preferably 100% pure. Polypeptides comprising a disintegrin domain can be prepared synthetically or prepared by recombinant expression.

The term “substantially” as used herein means at least 75% unless otherwise indicated.

The methods disclosed herein for inhibiting viral infection of a host cell by administering an effective amount of one or more disintegrin polypeptide as described herein can be used on a variety of cell types, that is, any cell type that expresses an integrin. Cell types include, but are not limited to, epithelial, endothelial, fibroblast and neuronal cells. Epithelial cells include, but are not limited to, skin, corneal and retinal pigment epithelial cells. Fibroblast cells include, but are not limited to, skin, corneal, and cervical fibroblast cells. Host cells can also be tissue culture cells including, but not limited to, HeLa cells and CHO-K1 cells expressing nectin-1, HVEM and 3-OST-3 receptors. The cell type can be from any appropriate species, e.g., mammalian, such as a canine cell, an equine cell, a bovine cell, an ovine cell, a porcine cell a goat cell or a human cell. For example, when the subject is other than a human, the method can be used as a pre-clinical screen for in vivo efficacy prior to administration into human patients.

The methods disclosed herein for inhibiting viral infection of a host cell by administering an effective amount of one or more disintegrin polypeptide as described herein can be used inhibit a variety of viruses. The methods disclosed herein are applicable to any virus that uses a host cell integrin for infection. The infection step the virus can use the host cell integrin includes, but is not limited to, viral entry, signaling, internalization, and transport. [55] Consequently, while not wishing to be held by theory, the methods provided herein can be used to inhibit viral infection by blocking the virus's use of the host cell integrin used at any stage of the infection process including, but not limited to, viral entry, signaling, internalization, and transport.

The virus can be an adenovirus. In humans, there are 55 accepted human adenovirus types (HAdV-1 to 55) in seven species (Human adenovirus A to G):
A: 12, 18, 31
B: 3, 7, 11, 14, 16, 21, 34, 35, 50, 55
C: 1, 2, 5, 6
D: 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54
E: 4
F: 40, 41
G: 52

Adenovirus can infect epithelial cells, e.g. mucoepithelial cells.

Different adenoviral types/serotypes are associated with different human conditions:
respiratory disease (mainly species HAdV-B and C)
conjunctivitis (HAdV-B and D)
gastroenteritis (HAdV-F serotypes 40 and 41)

When not restricting the subject to human viruses, Adenoviridae can be divided into five genera: Mastadenovirus, Aviadenovirus, Atadenovirus, Siadenovirus and Ichtadenovirus.

Two types of canine adenoviruses are well known, type 1 and 2. Type 1 causes infectious canine hepatitis, a potentially fatal disease involving vasculitis and hepatitis. Type 1 infection can also cause respiratory and eye infections. Canine adenovirus 2 (CAdV-2) is one of the potential causes of kennel cough. Core vaccines for dogs include attenuated live CAdV-2, which produces immunity to CAdV-1 and CAdV-2. CAdV-1 was initially used in a vaccine for dogs, but corneal edema was a common complication.

Adenoviruses are also known to cause respiratory infections in horses, cattle, pigs, sheep, and goats. Equine adenovirus 1 can also cause fatal disease in immunocompromised Arabian foals, involving pneumonia and destruction of pancreatic and salivary gland tissue.

The virus can be a Herpes virus. The Herpesviridae are a large family of DNA viruses that cause diseases in animals, including humans. The members of this family are also known as herpesviruses. They are divided into three main sub classes; alpha, beta and gamma.

The virus can be a HHV-1 or HHV-2, also known as Herpes Simplex Virus (HSV-1 or HSV-2), members of the alpha class. Cultured cells that can be infected by HSV include CHO-K1 cells expressing HSV-1 gD receptors (for example, CHO-K1 cells expressing either nectin-1, nectin-2, HVEM or 3-OST-3). Cultured cells further include, but are not limited to, HeLa, Vero, and 293T cells, which can express all three receptors. A variety of cells can be infected by HSV, including, but not limited to, epithelial cells such as mucoepithelial cells, corneal fibroblasts and retinal pigment epithelial (RPE) cells, neurons, and T-lymphocytes.

HSV is known to cause oral and genital herpes.

HHV-3, also known as Varicella zoster virus (VZV), a member of the alpha class. The virus can infect endothelial cells (including mucoendothelial cells, keratinocytes and the like), T cells, dendritic cells and neurons.

Primary VZV infection results in chickenpox (varicella), which may rarely result in complications including encephalitis or pneumonia. VZV remains dormant in the nervous system of the infected person (virus latency), in the trigeminal and dorsal root ganglia and can reactivate later in life producing a disease known as herpes zoster or shingles. Complications of shingles include postherpetic neuralgia, zoster multiplex, myelitis, herpes ophthalmicus, or zoster sine herpete.

HHV-4, also known as Epstein-Barr virus (EBV) and lymphocryptovirus, is a member of the gamma class. The virus can infect B cells and epithelial cells. It can also infect T cells, natural killer cells, and smooth muscle cells. [56]

EBV is Epstein-Barr virus occurs worldwide and causes infectious mononucleosis (glandular fever). There is also strong evidence that the virus has a primary role in the pathogenesis of multiple autoimmune diseases, particularly dermatomyositis, systemic lupus erythematosus, rheumatoid arthritis, Sjogren's syndrome, HIV-associated hairy leukoplakia, and multiple sclerosis, and may also be associated with type 1 diabetes mellitus. It is also known to cause several lymphoproliferative disorders and cancers, particularly Hodgkin's disease, Burkitt's lymphoma, post-transplant lymphoproliferative syndrome (PTLD), nasopharyngeal carcinoma, and central nervous system lymphomas associated with HIV

HHV-5, also known as Cytomegalovirus (CMV), is a member of the beta class. The virus can infect monocytes, lymphocytes, and epithelial cells.

HCMV (human CMV) can cause an infectious mononucleosis-like syndrome,^{[9]} and retinitis. HCMV infection is important to certain high-risk groups. Major areas of risk of infection include pre-natal or postnatal infants and immunocompromised individuals, such as organ transplant recipients, persons with leukemia, or those infected with human immunodeficiency virus (HIV). In HIV infected persons, HCMV is considered an AIDS-defining infection, indicating that the T-cell count has dropped to low levels. CMV infection has been linked to high blood pressure in mice, and suggests that the result of CMV infection of blood vessel endothelial cells (EC) in humans is a major cause of atherosclerosis. [57] Researchers also found that when the cells were infected with CMV, they created a protein called renin that is known to contribute to high blood pressure.

HHV-6, also known as Roseolovirus or Herpes lymphotropic virus, is a member of the beta class. There are two subtypes of HHV-6 termed HHV-6A and HHV-6B. The virus infects T cells. Sixth disease (roseola infantum or exanthem subitum) T cells. Respiratory and close contact?

Primary HHV-6 infections usually cause fever, with exanthem subitum (roseola infantum) being observed in 10% of cases. HHV-6 primary infections are associated with several more severe complications, such as encephalitis, lymphadenopathy, myocarditis and myelosuppression.

HHV-6 re-activation can lead to graft rejection, often in consort with other betaherpesviridae. Likewise in HIV/AIDS, HHV-6 re-activations cause disseminated infections leading to end organ disease and death. Although up to 100% of the population are exposed (seropositive) to HHV-6, most by 3 years of age, there are rare cases of primary infections in adults and has been linked to several central nervous system-related disorders. HHV-6 has been reported in multiple sclerosis patients and has been implicated as a co-factor in several other diseases, including chronic fatigue syndrome, fibromyalgia, AIDS, and temporal lobe epilepsy.

HHV-7, also a member of the beta class, often acts together with HHV-6, and the viruses together are sometimes referred to by their genus, Roseolovirus. The virus infects T cells.

There are indications that HHV-7 can contribute to the development of drug-induced hypersensitivity syndrome, encephalopathy, hemiconvulsion-hemiplegia-epilepsy syndrome, hepatitis infection, postinfectious myeloradiculoneuropathy, pityriasis rosea, and the reactivation of HHV-4, leading to “mononucleosis-like illness”. HHV-7 re-activation can lead to graft rejection.

HHV-8, also known as Kaposi's sarcoma-associated herpesvirus (KSHV), a type of rhadinovirus and a member of the gamma class. The virus infects lymphocytes, including B-cells, and epithelial cells.

HHV-8 causes Kaposi's sarcoma, primary effusion lymphoma, and some types of multicentric Castleman's disease B cell.

Other viruses that rely on integrins for cellular entry include Human Papilloma Virus (HPV)[58], Human metapneumovirus[59], Hantavirus, Picornovirus, Rotavirus, West Nile virus, foot-and-mouth disease virus, and ebola virus.

In an embodiment, the one or more disintegrin polypeptide as described herein can be used to treat a subject to either reduce (therapeutic) or prevent (prophylactic) the occurrence of a viral infection or to treat a viral infection to reduce the viremia. The one or more disintegrin polypeptides can be combined with other therapeutic agents for combination therapy. The polypeptide and other agent can be administered or contacted (in vitro) sequentially or simultaneously. The subject can be a mammal. The mammal can be a human. The mammal can be, for example, canine, feline, equine, bovine, ovine, murine, porcine, caprine, rodent, lagomorph, lupine, and ursine.

As used herein, “treating” refers to the administration of an agent (for example, a polypeptide comprising a disintegrin domain, an antiviral drug, or a vaccine) to a subject. Although it is preferred that treating a condition, such as a viral infection, will in all instances result in an improvement of the condition, the term “treating” as used herein does not indicate, imply, or require that the administration of the agent will always be successful in reducing or ameliorating symptoms associated with any particular condition.

As used herein, “administration” or “administer” or “administering” refers to dispensing, applying, or tendering an agent (for example a polypeptide comprising a disintegrin domain or an antiviral agent) to a subject. Administration may be performed using any of a number of methods known in the art. For example, injection of an agent can be intravenous, intraperitoneal, subcutaneous or intramuscular and the like.

As used herein, “IC50” refers to the concentration of a composition at which 50% inhibition of viral entry or viral infection is achieved for a host cell.

As used herein, “ED50” refers to the dose of a pharmaceutical composition at which 50% inhibition of viral entry or viral infection is achieved in a subject.

As used herein, “therapeutic composition” refers to a formulation suitable for administration to an intended animal or human subject for therapeutic purposes, and that contains at least one polypeptide of this invention, and at least one pharmaceutically acceptable carrier or excipient. The term “pharmaceutically acceptable” indicates that the identified material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectibles. Techniques for formulation and administration may be found, for example, in Remington's Pharmaceutical Sciences, (18th ed., Mack Publishing Co., Easton Pa., 1990).

As used herein, “about” means in quantitative terms plus or minus 10% unless indicated otherwise.

As used herein, “combination” refers to any association between or among two or more items. The combination can be two or more separate items, such as two compositions of matter or two collections. It can be a mixture thereof, such as a single mixture of the two or more items, or any variation thereof.

An effective concentration of a disintegrin can be determined by the skilled artisan using the methods disclosed herein. An effective concentration of CN can be at least 125 nM, at least 250 nM, at least 500 nM, at least 1 μM, at least 2.5 μM, at least 5 μM, or at least 10 μM or or ranges therebetween, e.g. from about 125 nM to about 50 μM. An effective concentration of CN can be at least 0.085 nM, at least 0.17 nM, at least 0.34 nM, at least 0.68 nM, at least 1.3 nM, at least 2.75 nM, at least 5.5 nM, at least 11 nM, or at least 22 nM. An effective concentration of VCN can be at least 2 μM, at least 5 μM, or at least 10 μM. An effective amount or effective dosage of a disintegrin can be at least 0.01 mg/kg, at least 0.02 mg/kg, at least 0.05 mg/kg, at least 0.1 mg/kg, at least 0.2 mg/kg, at least 0.5 mg/kg, at least 1 mg/kg, at least 2 mg/kg, at least 5 mg/kg, at least 10 mg/kg, at least 20 mg/kg, or at least 50 mg/kg or ranges therebetween, e.g. from about 0.01 mg/kg to about 100 mg/kg.

As used herein, “effective amount” refers to a dose sufficient to provide a concentration high enough to impart a beneficial effect on the recipient thereof. An “effective amount” may be determined by conducting clinical trials in accordance with generally accepted or legal guidelines. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet and general health of the subject, and like factors well known in the medical arts and sciences. Various general considerations taken into account in determining the “therapeutically effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 18th ed., Mack Publishing Co., Easton, Pa., 1990.

Therapeutic compositions of polypeptides comprising a disintegrin domain as described herein will typically be used in therapy for human subjects. However, therapeutic compositions of polypeptides comprising a disintegrin domain as described herein may also be used to treat similar or identical indications in other animal subjects, and can be administered by different routes, including injection (i.e. parenteral, including intravenous, intraperitoneal, subcutaneous, and intramuscular), oral, transdermal, transmucosal, rectal, or inhalant. Such dosage forms should allow the therapeutic composition of a polypeptide comprising a disintegrin domain to reach host cells. Other factors are well known in the art, and include considerations such as toxicity and dosage forms that retard the therapeutic composition of a polypeptide comprising a disintegrin domain from exerting its effects. Techniques and formulations generally may be found in Remington: The Science and Practice of Pharmacy, 21st edition, Lippincott, Williams and Wilkins, Philadelphia, Pa., 2005 (hereby incorporated by reference herein).

In some embodiments, therapeutic compositions will comprise pharmaceutically acceptable carriers or excipients, such as fillers, binders, disintegrants, glidants, lubricants, complexing agents, solubilizers, and surfactants, which may be chosen to facilitate administration of the therapeutic composition of a polypeptide comprising a disintegrin domain by a particular route. Examples of carriers include calcium carbonate, calcium phosphate, various sugars such as lactose, glucose, or sucrose, types of starch, cellulose derivatives, gelatin, lipids, liposomes, nanoparticles, and the like. For example, Swenson et al. describes use of intravenous delivery of contortrostatin in liposomes for therapy of breast cancer [51]. Carriers also include physiologically compatible liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-.delta.-cyclodextrin), polysorbates (e.g. polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

In some embodiments, oral administration may be used. Pharmaceutical preparations for oral use can be formulated into conventional oral dosage forms such as capsules, tablets, and liquid preparations such as syrups, elixirs, and concentrated drops. Therapeutic compositions of polypeptides comprising a disintegrin domain as described herein may be combined with solid excipients, optionally grinding a resulting mixture, and processing the mixture of granules, after adding suitable auxiliaries, if desired, to obtain, for example, tablets, coated tablets, hard capsules, soft capsules, solutions (e.g. aqueous, alcoholic, or oily solutions) and the like. Suitable excipients are, in particular, fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone); oily excipients, including vegetable and animal oils, such as sunflower oil, olive oil, or codliver oil. The oral dosage formulations may also contain disintegrating agents, such as cross-linked polyvinylpyrrolidone, agar, or alginic acid, or a salt thereof such as sodium alginate; a lubricant, such as talc or magnesium stearate; a plasticizer, such as glycerol or sorbitol; a sweetening agent such as sucrose, fructose, lactose, or aspartame; a natural or artificial flavoring agent, such as peppermint, oil of wintergreen, or cherry flavoring; or dye-stuffs or pigments, which may be used for identification or characterization of different doses or combinations. Also provided are dragee cores with suitable coatings. For this purpose, concentrated sugar solutions may be used, which may optionally contain, for example, gum arabic, talc, poly-vinylpyrrolidone, carbopol gel, polyethylene glycol, and/or titanium dioxide, lacquer solutions, and suitable organic solvents or solvent mixtures.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compound may be dissolved or suspended in suitable liquids, such as fatty oils, liquid paraffin, or liquid polyethylene glycols.

In some embodiments, injection (parenteral administration) may be used, e.g., intramuscular, intravenous, intraperitoneal, and/or subcutaneous. Therapeutic compositions of polypeptides comprising a disintegrin domain as described herein for injection may be formulated in sterile liquid solutions, preferably in physiologically compatible buffers or solutions, such as saline solution, Hank's solution, or Ringer's solution. Dispersions may also be prepared in non-aqueous solutions, such as glycerol, propylene glycol, ethanol, liquid polyethylene glycols, triacetin, and vegetable oils. Solutions may also contain a preservative, such as methylparaben, propylparaben, chlorobutanol, phenol, sorbic acid, thimerosal, and the like. In addition, the therapeutic compositions of polypeptides comprising a disintegrin domain may be formulated in solid form, including, for example, lyophilized forms, and redissolved or suspended prior to use.

In some embodiments, transmucosal, topical or transdermal administration may be used. In such formulations of therapeutic compositions of polypeptides comprising a disintegrin domain as described herein, penetrants appropriate to the barrier to be permeated are used. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal). Therapeutic compositions of compositions of polypeptides comprising a disintegrin domain as described herein for topical administration may be formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C12). In some embodiments, carriers are selected such that the active ingredient is soluble. Emulsifiers, stabilizers, humectants and antioxidants may also be included as well as agents imparting color or fragrance, if desired. Creams for topical application are preferably formulated from a mixture of mineral oil, self-emulsifying beeswax and water in which mixture the active ingredient, dissolved in a small amount of solvent (e.g., an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will be continuous rather than intermittent throughout the dosage regimen.

In some embodiments, therapeutic compositions of polypeptides comprising a disintegrin domain are administered as inhalants. Therapeutic compositions of polypeptides comprising a disintegrin domain as described herein may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like.

The amounts of therapeutic compositions of polypeptides comprising a disintegrin domain as described herein to be administered can be determined by standard procedures taking into account factors such as the compound activity (in vitro, e.g. the compound IC50 vs. target, or in vivo activity in animal efficacy models), pharmacokinetic results in animal models (e.g. biological half-life or bioavailability), the age, size, and weight of the subject, and the disorder associated with the subject. The importance of these and other factors are well known to those of ordinary skill in the art. Therapeutic compositions containing disintegrins should comprise at a minimum an amount of the disintegrin effective to achieve the desired effect (i.e., inhibit or reduce viral infection in a subject) and include a buffer, salt, and/or suitable carrier or excipient. Generally, in these therapeutic compositions, disintegrins are present in an amount sufficient to provide about 0.01 mg/kg to about 50 mg/kg per day, preferably about 0.1 mg/kg to about 5.0 mg/kg per day, and most preferably about 0.1 mg/kg to about 0.5 mg/kg per day.

The therapeutic compositions of polypeptides comprising a disintegrin domain as described herein may also be used in combination with other therapies for treating the same disease. Such combination use includes administration of the therapeutic compositions of polypeptides comprising a disintegrin domain and one or more other therapeutics at different times, or co-administration of the compound and one or more other therapies. In some embodiments, dosage may be modified for one or more of the therapeutic compositions of polypeptides comprising a disintegrin domain as described herein or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a therapeutic composition of a polypeptide comprising a disintegrin domain as described herein, or at the same time as a therapeutic composition of a polypeptide comprising a disintegrin domain as described herein. Use in combination also includes use with a therapy or medical procedure that is administered once or infrequently, such as surgery, along with a therapeutic composition of a polypeptide comprising a disintegrin domain as described herein administered within a short time or longer time before or after the other therapy or procedure. In some embodiments, the present invention provides for delivery of a therapeutic composition of a polypeptide comprising a disintegrin domain as described herein and one or more other drug therapeutics delivered by a different route of administration or by the same route of administration. The use in combination for any route of administration includes delivery of a therapeutic composition of a polypeptide comprising a disintegrin domain as described herein and one or more other drug therapeutics delivered by the same route of administration together in any formulation, including formulations where the two therapeutic compositions of polypeptides comprising a disintegrin domain are chemically linked in such a way that they maintain their therapeutic activity when administered. In one aspect, the other drug therapy may be co-administered with a therapeutic composition of a polypeptide comprising a disintegrin domain as described herein. Co-administration of separate formulations includes co-administration by delivery via one device, for example the same inhalant device, the same syringe, etc., or administration from separate devices within a short time of each other. Co-formulations of a therapeutic composition of a polypeptide comprising a disintegrin domain as described herein and one or more additional drug therapies delivered by the same route includes preparation of the materials together such that they can be administered by one device, including the separate compounds combined in one formulation.

For example, therapeutic compositions of polypeptides comprising a disintegrin domain can be combined with other anti-viral drugs. The therapeutic compositions of polypeptides comprising a disintegrin domain as described herein may be used with other chemotherapeutic drugs for the treatment of viral diseases, such as, without limitation, Rifampin, Ribavirin, Pleconaryl, Cidofovir, Acyclovir, Pencyclovir, Gancyclovir, Valacyclovir, Famciclovir, Foscarnet, Vidarabine, Amantadine, Zanamivir, Oseltamivir, Resquimod, antiproteases, pegylated interferon, anti HIV proteases (e.g. lopinivir, saquinivir, amprenavir), HIV fusion inhibitors, nucleotide HIV RT inhibitors (e.g., AZT, Lamivudine, Abacavir), non-nucleotide HIV RT inhibitors, Doconosol, Interferons, Butylated Hydroxytoluene (BHT) and Hypericin. Such additional factors and/or agents may be included in the therapeutic composition, for example, to produce a synergistic effect with the polypeptides of the invention.

In another embodiment, the one or more polypeptide comprising a disintegrin domain can be combined with the use of one or more vaccines, either therapeutic or prophylactic. Such vaccines include, but are not limited to, vaccines against Adenovirus, Herpesvirus, Human Papilloma Virus (HPV), Human metapneumovirus, Hantavirus, Picornovirus, Rotavirus, West Nile virus, foot-and-mouth disease virus, and ebola virus. For HSV, vaccines currently in clinical trials include Herpevac (GSK), a vaccine against HSV-2. Another is d15-29 (aka ACAM-529; Sanofi Pasteur), a replication-defective mutant virus that has proved successful both in preventing HSV-2/HSV-1 infections, and ImmunoVEX (BioVex).

### EXAMPLE 1

### Materials and Methods

Preparation of rCN, VCN, and MAPs. The DNA sequences of rCN, VCN, and the MAPs were cloned into pET32a vector downstream of thioredoxin A (TrxA) using a BglII/NcoI set of restriction enzymes. The forward primers for the coding sequences of the rCN, VCN, and the MAPs introduced a unique TEV protease cleavage site, which made possible the removal of thioredoxin during purification. To build the VCN construct, the nucleotides encoding the C-terminal tail of echistatin were added to CN via an elongated reverse primer. The primers used for rCN were: forward—5′gttccagatctcgagaatctttacttccaaggagacgctcctgcaaatccgtgctgcga3′, and reverse—5′gttattcgccatggcttaggcatggaagggatttctgggacagccagcaga3′. The primers used for VCN were: forward—5′gttccagatctcgagaatctttacttccaaggagacgctcctgcaaatccgtgctgcga3′, and reverse—5′gttattcgccatggcttaagtagctggacccttgtggggatttctgggacagccagcagatatgcc3′. The plasmids were initially amplified in DH5α E. coli, purified and sequenced, and then transferred into Origami B (DE3) E. coli. Multiple cultures were established for each construct from individual colonies of transformed BL21 (DE3), AD494 (DE3) or Origami B (DE3) in LB media containing either carbenicillin (50 μg/mL) alone, or carbenicillin (50 μg/mL) plus kanamycin (15 μg/mL) or carbenicillin (50 μg/mL) plus tetracycline (12.5 μg/mL), plus kanamycin (15 μg/mL) and grown at 37° C. and 250 rpm in a shaker-incubator until they reached an OD₆₀₀ of 0.6-1. At this point, the cells were induced in 1 mM IPTG (isopropyl-1-β-D-thio-1-galactopyranoside) and incubated for another 4-5 hours at 37° C. and 250 rpm. At the end of the induction period, the cells were pelleted at 4000×g and lysed in a microfluidizer (Microfluidics M-110L, Microfluidics, Newton, Mass.). The operating conditions of the microfluidizer included applied pressures of 14,000-18,000 psi, bacterial slurry flow rates of 300-400 ml per minute and multiple passes of the slurry through the processor. The lysate insoluble cellular debris was removed by centrifugation (40,000×g) and the soluble material containing either Trx-rCN or Trx-VCN collected. The expressed fusion proteins in the collected soluble lysates were then proteolysed by incubation with recombinant TEV protease overnight at room temperature which efficiently cleaved off rCN or VCN from TrxA as monitored by SDS-PAGE (sodium dodecyl sulfate-polyacrylamide gel electrophoresis). When proteolysis was complete, the proteolyzed lysates were passed through a 0.22 μm filter, diluted 1:100 in ddH₂O, ultrafiltrated through a 50,000 MWCO cartridge (Biomax50, Millipore) and then reconcentrated against a 5,000 MWCO cartridge (Biomax5, Millipore) using a tangential flow ultrafiltration device (Labscale TFF system, Millipore).

Purification of recombinant disintegrins was done by C18-reverse phase HPLC using the standard elution conditions previously employed for the purification of native CN [26]. The filtrated lysates processed as described above were loaded onto a Vydac C 18 column (218TP54, Temecula, Calif.). A ten-minute rinse (at 5 ml/min) of the column with an aqueous solution containing 0.1% TFA was followed by a linear gradient (0-100%) elution over 150 min in a mobile phase containing 80% acetonitrile and 0.1%TFA. rCN starts eluting in 30% acetonitrile, while VCN elutes in 35% acetonitrile.

Cells, Viruses and contortrostatin. Wild-type Chinese hamster ovary (CHO-K1) cells were grown in Ham's F12 (Invitrogen Corp., Carlsbad, Calif., USA) supplemented with 10% fetal bovine serum (FBS), while African green monkey kidney (Vero) cells were grown in Dulbecco's modified Eagles medium (DMEM; Intitrogen Corp.) supplemented with 5% FBS. CHO-Ig8 cells, obtained by the stable transfection of CHO-K1 cells with pMLP01, express the Escherichia coli lac Z gene under control of the HSV-1 ICP4 promoter. Cultures of HeLa cells were grown in L-glutamine-containing DMEM (Invitrogen Corp.) supplemented with 10% FBS. As previously described, cultures of human corneal fibroblasts (CF) were grown in DMEM media supplemented with 10% FBS and 5% calf serum. Recombinant β-galactosidase-expressing HSV-1 (KOS) gL86 was used. The viral stocks were propagated at low multiplicity of infection in complementing cell lines, tittered on Vero cells and stored at −80° C.

Materials. The stocks (1 mg/ml) of contortrostatin (described as CN) and recombinant-VCN were prepared as described.[38, 52] and stored at −20° C. until used. Wild-type Chinese hamster ovary (CHO-K1) cells were grown in Ham's F12 (Invitrogen Corp, Carlsbad, Calif.) supplemented with 10% fetal bovine serum (FBS), while African green monkey kidney (Vero) cells were grown in Dulbecco's Modified Eagles Medium (DMEM) (Invitrogen Corp.) supplemented with 5% FBS. Cultures of HeLa and retinal pigment epithelial (RPE) cells were grown in L-glutamine containing DMEM (Invitrogen Corp.) supplemented with 10% FBS. Recombinant -galactosidase-expressing HSV-1(KOS) gL86 and CMV wild type strain Ad169 were used. The viral stocks were propagated at low multiplicity of infection (MOI) in complementing cell lines, tittered on Vero cells and stored at −80° C. The plasmids expressing HVEM (pBec10), nectin-1 (pBG38) and 3-OST-3 (pDS43) were prepared as described [12].

Viral Entry Assay. Viral entry assays were based on quantitation of β-galactosidase expressed from the viral genome in which β-galactosidase expression is inducible by HSV infection. Cells were transiently transfected in 6-well tissue culture dishes, using Lipofectamine 2000 (In vitrogen Corp) with plasmids expressing HSV-1 entry receptors (necitn-1, HVEM and 3-OST-3 expression plasmids) at 1.5 μg per well in 1 ml. At 24 hr post-transfection, cells were re-plated in 96-well tissue culture dishes (2×10⁴ cells per well) at least 16 hr prior to infection. Cells were washed and exposed to serially dilute pre-incubated virus with neem bark extract (NBE) or with 1×PBS at two fold dilutions for 2 hr at room temperature. In a parallel experiment the cells were also pre-incubated with NBE for 2 hr at room temperature before infection with virus. Later the cells were washed with 1×PBS and allowed 6 hr at 37° C. before solubilization in 100 μl of PBS containing 0.5% NP-40 and the -galactosidase substrate, o-nitro-phenyl-D-galactopyranoside (ONPG; ImmunoPure, PIERCE, Rockford, Ill., 3 mg/ml). The enzymatic activity was monitored at 410 nm by spectrophotometry at several time points after the addition of ONPG in order to define the interval over which the generation of the product was linear with time. Microscopy was performed using 20×objective of the microscope (Nikon). The slide book version 3.0 was used for images. All experiments were repeated a minimum of three times.

Viral plaque assay. Confluent layers of RPE cells in glass bottom dishes were infected with CMV Ad169 strain in the presence and absence of CN at 0.01 PFU per cell for 2 hrs. This was followed by extensive but gentle washing three times with DMEM media followed by feeding cells with DMEM media. The cells were then incubated at 37° C. for thee days. Cells were then fixed using fixative buffer after 3 days for 30-45 min. The cells were washed three times with 1X PBS before RPE cells stained for Giemsa satin for additional 45 minutes. This was followed by 10 times washing with 1 X PBS to remove excess stain. The number of plaques formed were then visualized and quantified as indicated. In another protocol, confluent layers of HeLa cells (approximately 10⁶) in six-well dishes were infected with HSV-1 (804) strain at 0.01 PFU/celI in presence and absence of CN for 2 h at 37° C. After removal of inoculum, monolayers were overlaid with DMEM comaining 2.5% heat-inactivated calf serum and incubated at 37° C. At 24 h, the cells were fixed by using fixative buffer (2% formaldehyde and 0.2% glutaradehyde) at room temperature for 20 min, followed by Giemsa staining for 45 min. The cells were again washed 5× in PBS and the numbers of plaques were counted. The images were taken by using Nikon D-Eclipse-C1 microscope (Nikon Instruments Inc., Melville, N.Y., USA).

Virus-free cell-to-cell fusion assay. In this experiment, the CHO-KI cells (grown in F-12 Ham, Invitrogen Corp.) designated “effector” cells were co-transfected with plasmids expressing four HSV1 (KOS) glycoproteins, pPEP98 (gB), pPEP99 (gD), pPEP100 (gH) and pPEP101 (gL), along with the plasmid pT7EMCLuc that expresses firefly luciferase gene under the T7 promoter. Human CF considered as “target cells” were co-transfected with pCAGT7 that expresses T7 RNA polymerase using chicken actin promoter and CMV enhancer. The untreated effector cells expressing pT7EMCLuc and HSV-1 essential glycoproteins and the target CF cells expressing gD receptors transfected with T7 RNA polymerase were used as the positive control; CN-treated target cells were used for the test. For fusion, at 18 h post-transfection, the target and the effector cells were mixed together (1:1 ratio) and co-cultivated in 24-well dishes. The activation of the reporter luciferase gene was examined as a measure of cell fusion using reporter lysis assay (Promega Corporation, Madison, Wis., USA) at 74 h post-mixing.

### EXAMPLE 2

### Inhibition of HSV-1 Entry Into CHO-K1 By Contortrostatin

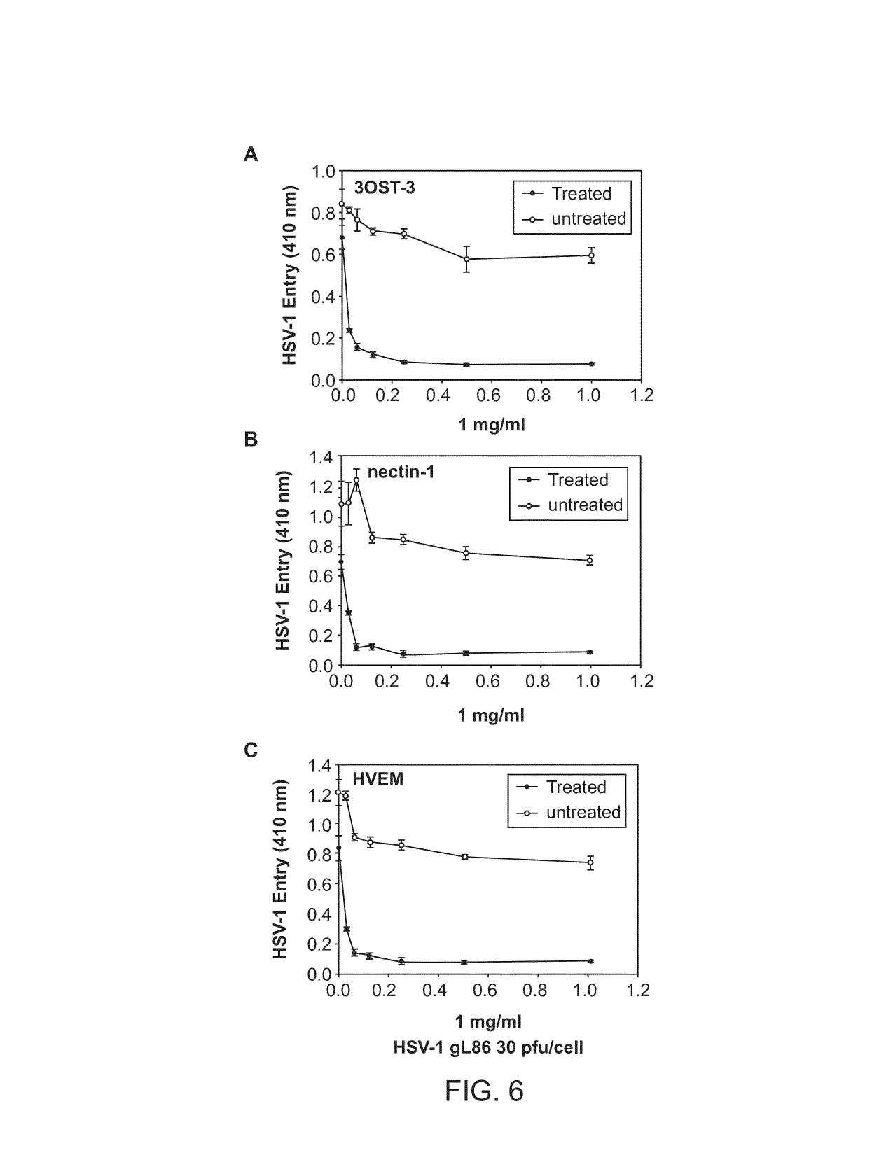

CN blocks herpes simplex virus type-1 (HSV-1) entry into CHO-K1 cells expressing gD receptors. CHO-K1 cells were transiently transfected in 6-well tissue culture dishes, using Lipofectamine 2000 (Invitrogen Corp., Carlsbad, Calif.) with plasmids expressing HSV-1 entry receptors (necitn-1, HVEM and 3-OST-3 expression plasmids) at 1.5 μg per well in 1 ml. At 24 hr post-transfection, cells were re-plated in 96-well tissue culture dishes (2 ×10⁴ cells per well) at least 16 hr prior to infection. β-galactosidase-expressing recombinant virus HSV-1 (KOS) HSV-1 gL86 (30 pfu/cell) was pre-incubated with CV-N at the concentration indicated in FIG. 6 or mock treated with 1 X phosphate saline buffer for 90 min at room temperature. After 90 min the virus was incubated with CHO-K1 cells expressing gD receptors: 3-OST-3 (A), nectin-1 (B) and HVEM (C) expressing cells (FIG. 6). After 6 hr at 37° C., the cells were washed, permeabilized with 100 μl of PBS containing 0.5% NP-40 and incubated with ONPG substrate (3.0 mg/ml) for quantitation of β-galactosidase activity expressed from the input viral genome. The enzymatic activity was measured at an optical density of 410 nm (OD 410).

FIG. 6 shows that CN blocked viral entry into CHO-K1 cells expressing 3-OST-3, nectin-1 or HVEM.

### EXAMPLE 3

### Inhibition of HSV-1 Infection of HeLa, Vero and 293T By Contortrostatin

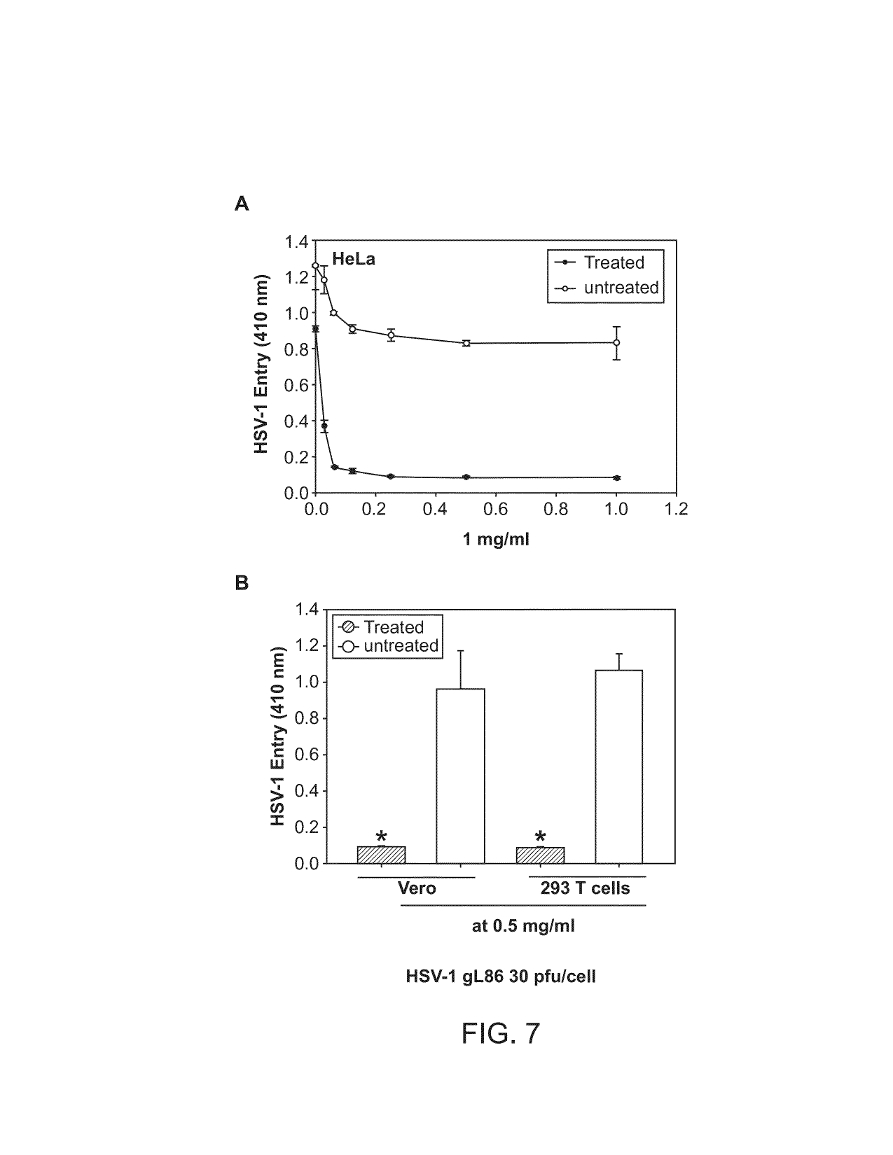

CN blocks herpes simplex virus type-1 (HSV-1) infection of natural target cells. HeLa, Vero and 293T cells were each grown to 100% confluency in 96-well plates. The β-galactosidase-expressing recombinant virus HSV-1 (KOS) HSV-1 gL86 (30 pfu/cell) was pre-incubated with CN at the concentration indicated in FIG. 7 or mock treated with 1 X phosphate saline buffer for 90 min at room temperature. After 6 hr, the cells were washed, permeabilized and incubated with ONPG substrate (3.0 mg/ml). The β-gal enzymatic activity was measured at an optical density of 410 nm (OD 410). Each value shown in FIG. 7 is the mean of three or more determinations (±SD). Mock treated HSV-1 with PBS was used as a control. HSV-1 infection of HeLa (panel A) and Vero and 293T cells (panel B) was blocked by CN. VCN also blocked HSV-1 infection of HeLa cells.

### EXAMPLE 4

### Inhibition of HSV Infection of CHO Igβ Cells By Contortrostatin Independent of Strain Type

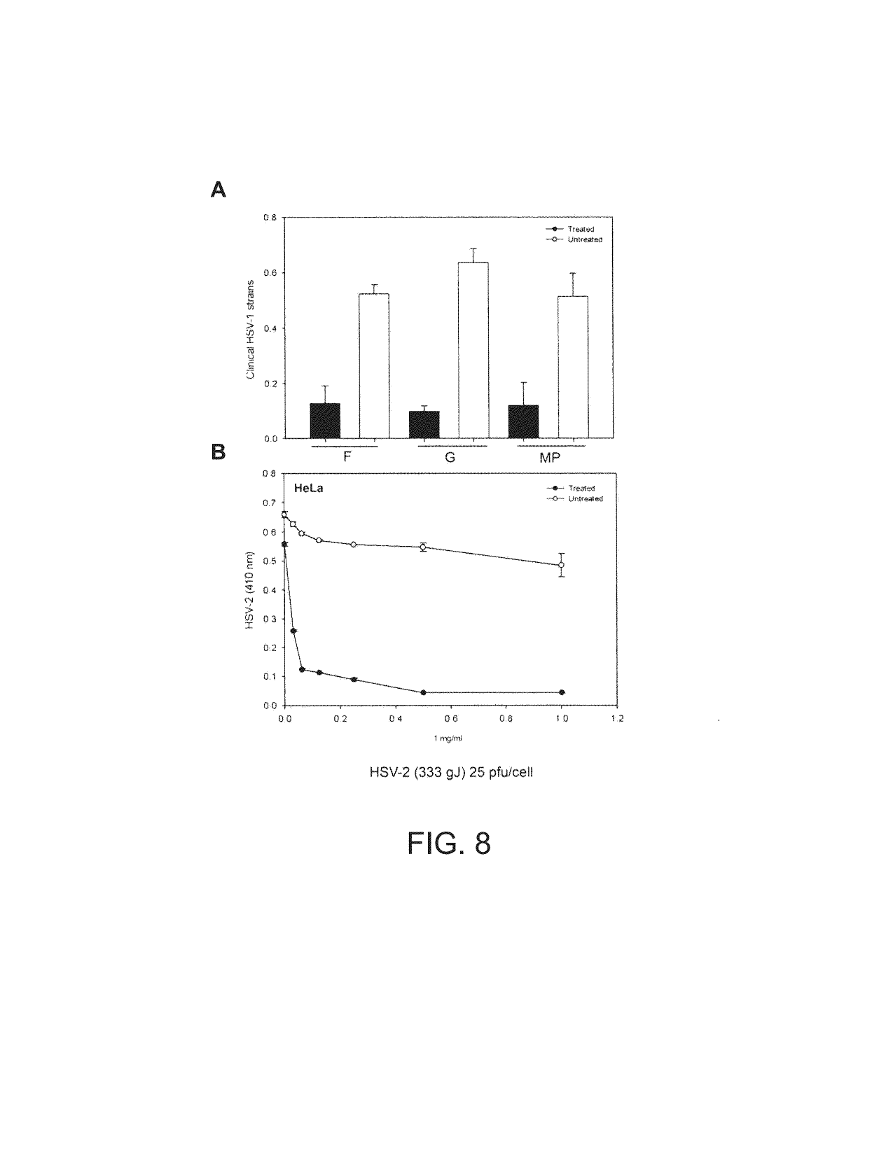

HSV-1 infection blocking activity of CN is not viral-strain specific. In this experiment different clinical strains of HSV-1 (F, G, and MP strains of HSV-1 [37] at 25 pfu/cell) were either pre-incubated with 1×PBS (control) or with CN at the concentrations indicated in FIG. 8, panel A for 90 min at room temperature. After 90 min of incubation the two pools of viruses were incubated with Nectin-1 receptor expressing CHO Igβ cells that express β-galactosidase upon viral entry as described. [12] The viral infection blocking was measured by ONPG assay (FIG. 8, panel A). Similar experiment was conducted using Lac Z encoded HSV-2 (333 gJ-reporter virus). HSV-2 is a genital herpes virus. A dose response curve shows that pre-incubation of HSV-2 with VCN (starting concentration 1 mg/ml) significantly inhibited HSV-2 infection of HeLa cells (FIG. 8, panel B). Each value shown is the mean of three or more determinations (±SD).

### EXAMPLE 5

### Inhibition of CMV Infection of RPE Cells By Contortrostatin

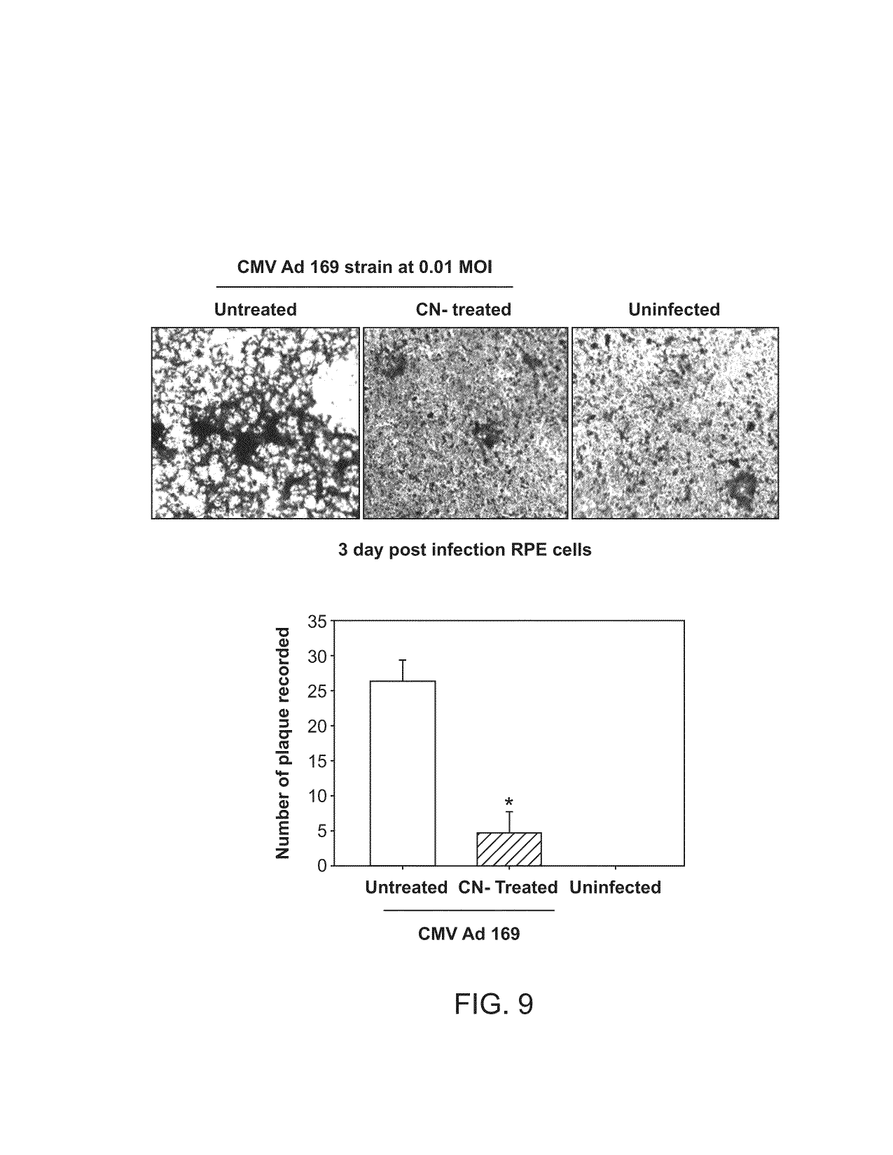

Cytomegalovirus (CMV-a member of betaherpesvirus subfamily) spread from cell to cell (plaque formation) was significantly blocked using CN. A wild type strain of CMV Ad169 was used. The virus was again either pre-incubated with 1×PBS (control) or with CN at 1 mg/ml for 120 min at room temperature. After 120 min of incubation the two pools of viruses were incubated on target retinal pigment epithelial (RPE) cells at MOI (multiplicity of infection; MOI) of 0.01 for additional 2 hrs at 37° C. This was followed by washing to remove unbound viruses and feeding cells with fresh DMEM media. The plates were kept for 3 days before the plaque formation was visually observed (upper panels) and quantified (lower panel) after fixing cells with buffer (FIG. 9). The upper middle panel shows that CN treated virus had significantly lower cytopathic effect (CPE) while PBS treated CMV had extensive plaque formation (CPE) (upper left panel). The uninfected RPE cells were used as an internal negative control (upper right panel). Based on visual evidence, the plaque formation was quantified in each group and was scored (lower panel). Again the CMV virion treated with CN significantly reduced the plaque formation in RPE cells. The size of plaques was also small compared to untreated CMV virions.

### EXAMPLE 6

### Inhibition of Herpes Simplex Virus-1 Infection of HeLa Cells By Contortrostatin, Vicrostatin, and ADAM15

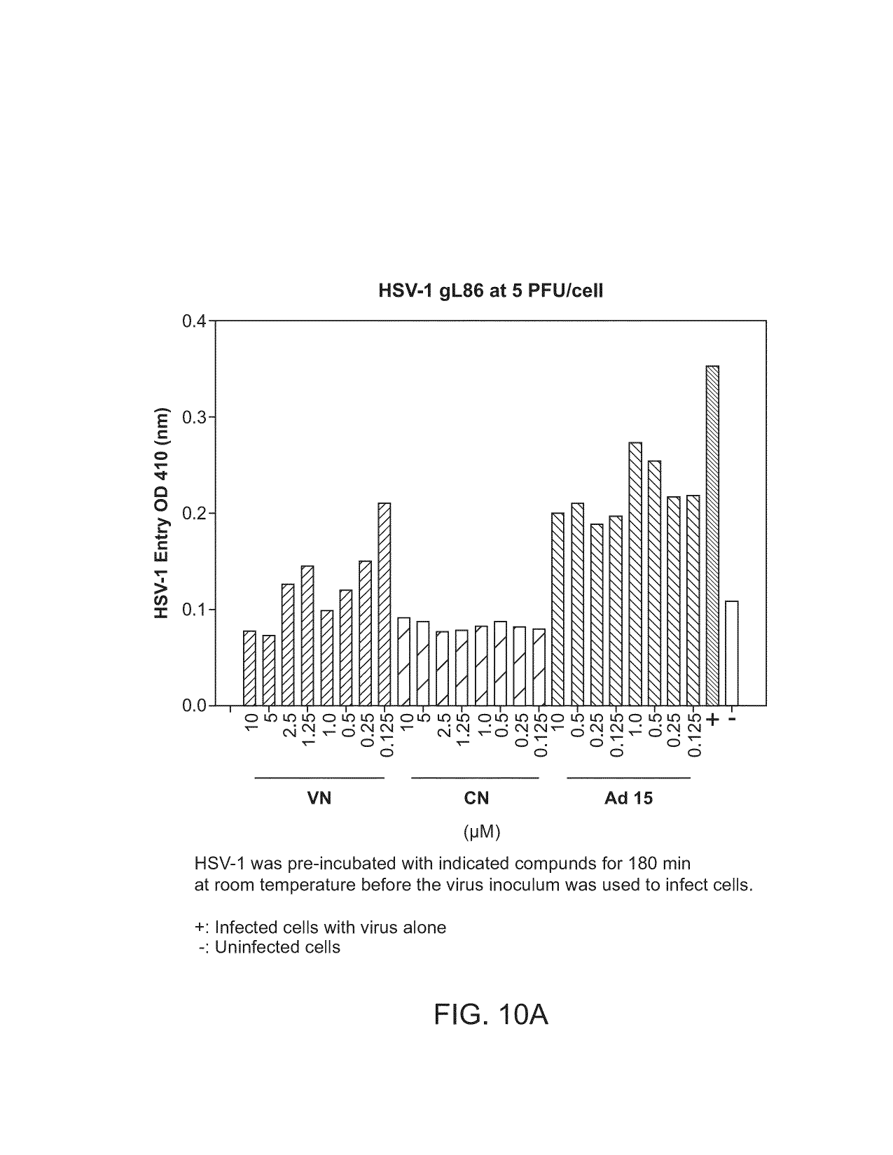

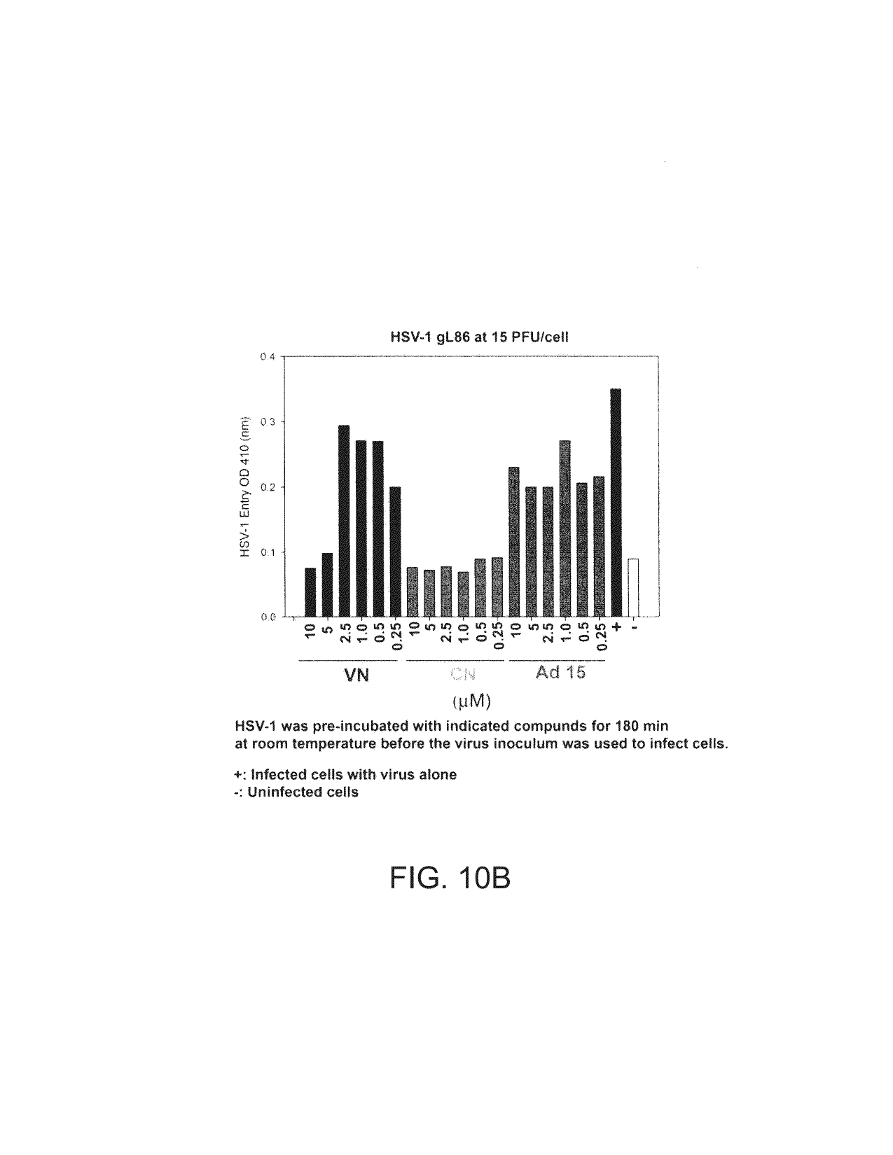

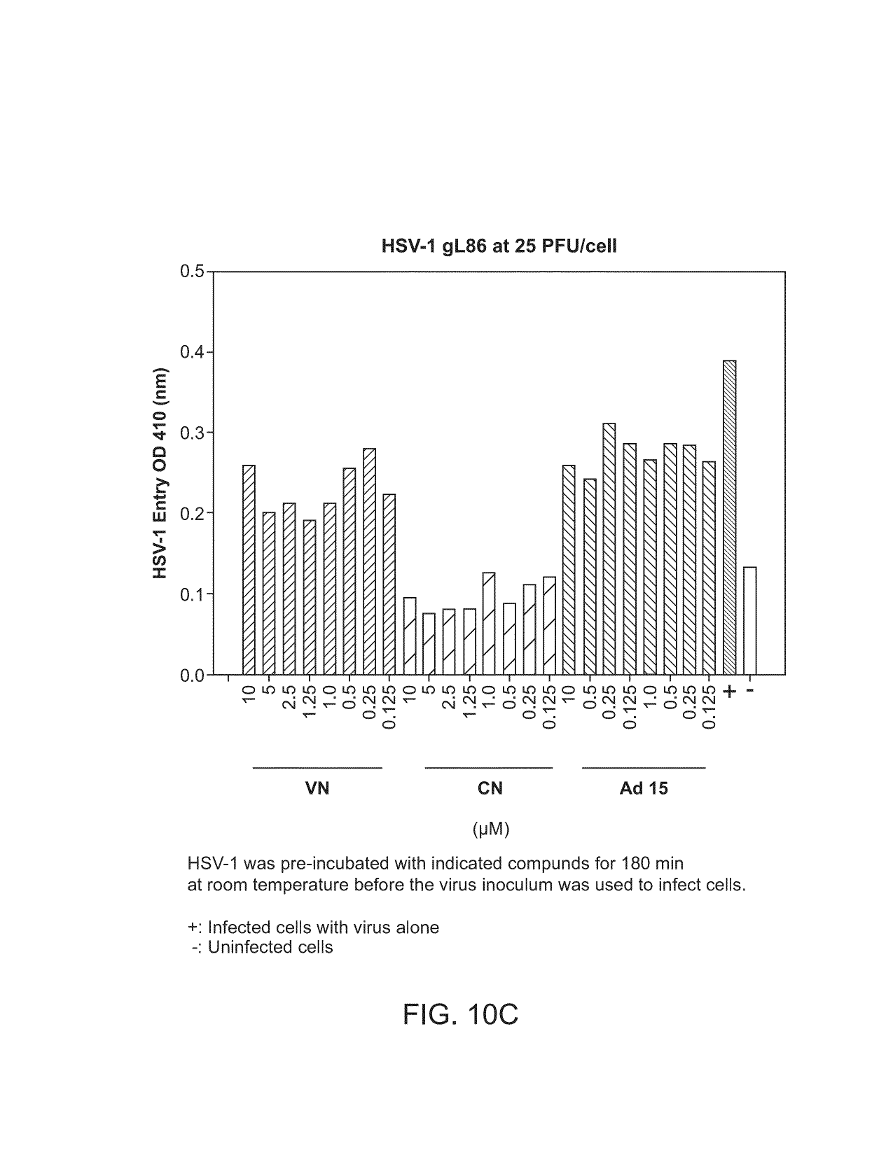

HeLa cells were each grown to 100% confluency in 96-well plates. The β-galactosidase-expressing recombinant virus HSV-1 (KOS) HSV-1 gL86 (30 pfu/cell) was pre-incubated at MOIs of 5 (FIG. 10, panel A), 15 (FIG. 10, panel B) and 25 (FIG. 10, panel C) plaque-forming units per cell (PFU) with VCN (VN), CN or ADAM15 (Ad15) at the concentrations indicated or mock treated with 1 X phosphate saline buffer for 180 min at room temperature. After 6 hr, the cells were washed, permeabilized and incubated with ONPG substrate (3.0 mg/ml). The β-gal enzymatic activity was measured at an optical density of 410 nm (OD 410). Mock treated HSV-1 with PBS was used as a negative control ((+) infected with virus alone; (−) uninfected control). HSV-1 infection of HeLa cells was blocked by CN >VCN >ADAM15.

### EXAMPLE 7

### CN Inhibition of Adenovirus Infection of HeLa Cells

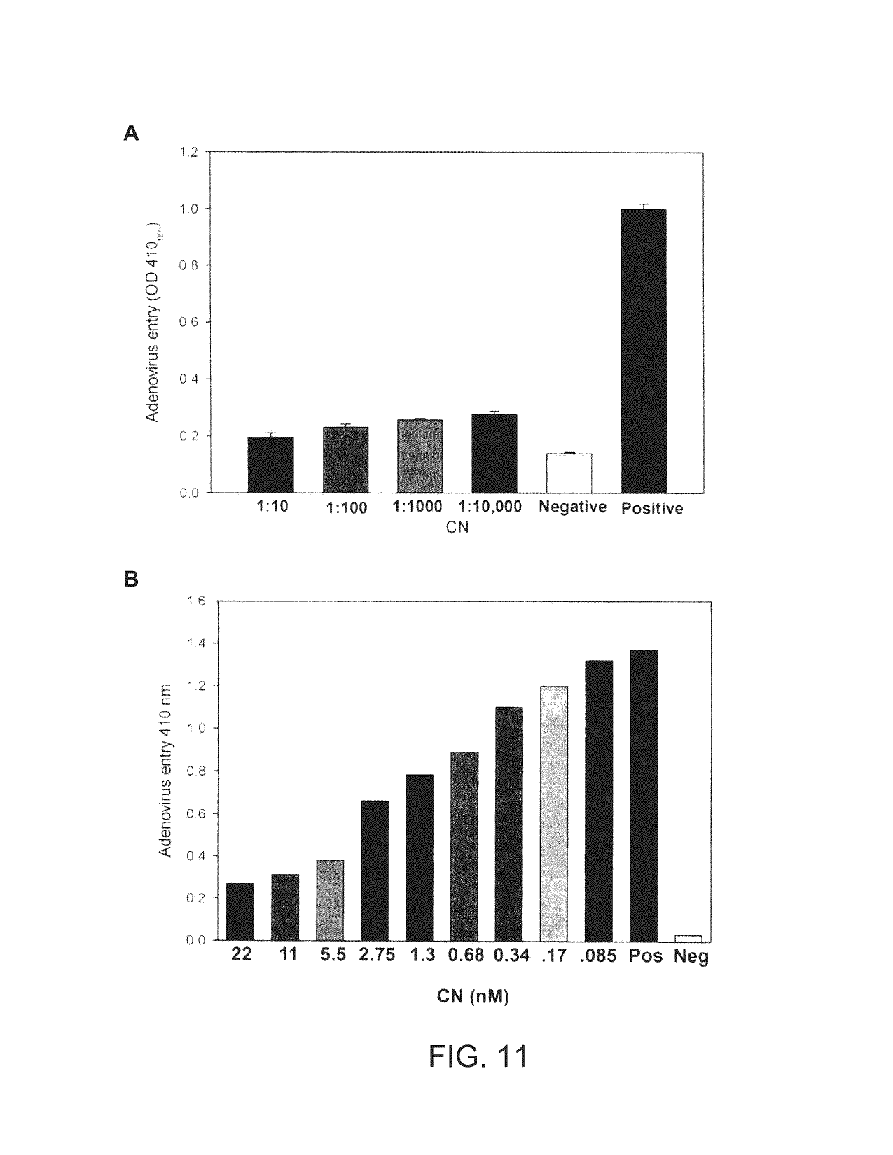

HeLa cells were infected with a β-galactosidase-expressing recombinant Adenovirus [60] were pre-incubated with CN at (1) the dilution indicated in FIG. 11, panel A (stock concentration 22 nM) or (2) the concentrations indicated in FIG. 11, panel B, or mock treated with 1 X phosphate saline buffer for 90 min at room temperature. After 12 hr, the cells were washed, permeabilized and incubated with ONPG substrate (3.0 mg/ml) for quantitation of β-galactosidase activity expressed from the input viral genome. Positive (Pos) infected with virus alone; Negative (Neg) uninfected control. The enzymatic activity was measured at an optical density of 410 nm (OD 410).

FIG. 11 shows that CN blocks infection of adenovirus into HeLa cells. The IC50 of CN was 2.75 nM. Similar experiments were performed with VCN and an IC50 of 21 μM was observed.

Consequently, CN and VCN have characteristics of a broad spectrum viral inhibitor.

### EXAMPLE 8

CN Significantly Inhibits HSV-1 Entry into gD Receptor Expressing CHO-K1 Cells

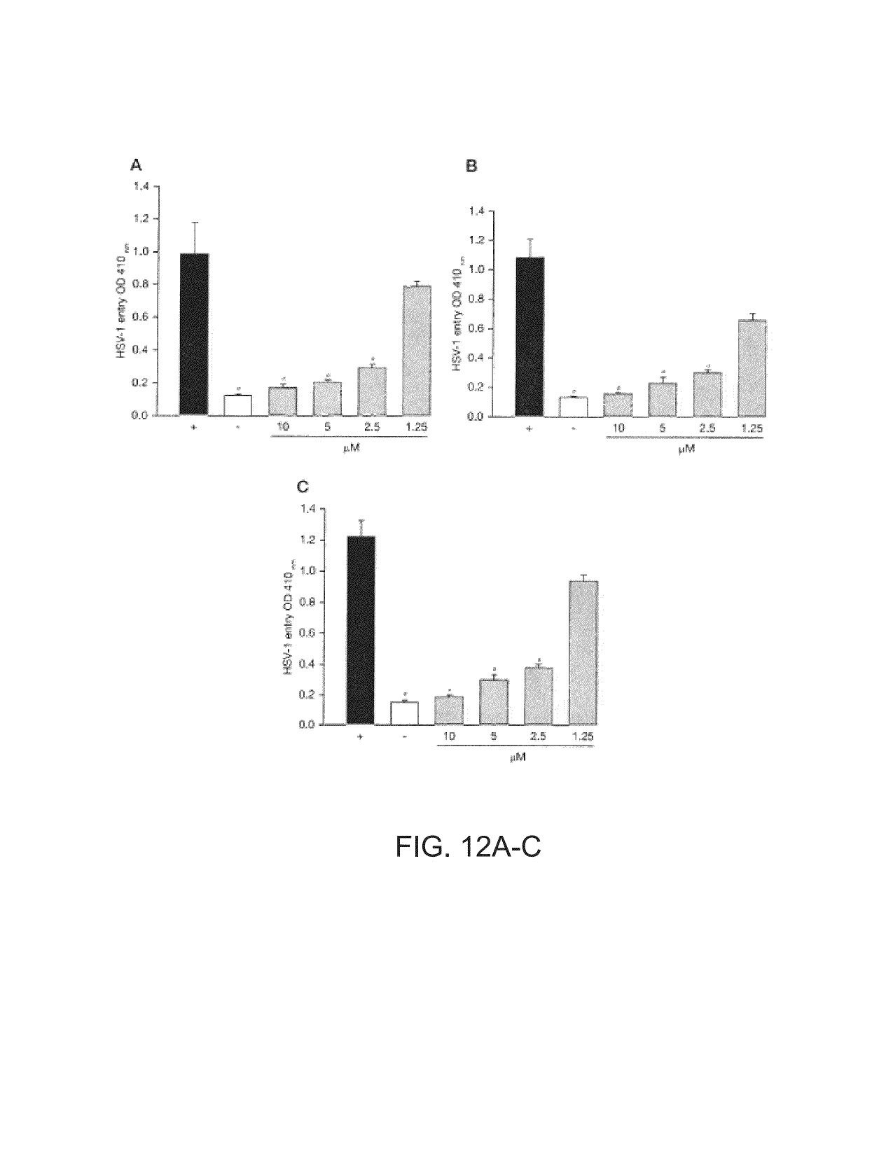

To determine the effect of CN on HSV-1 entry, we first tested the ability of HSV-1 in the presence and absence of CN to infect CHO-KI cells expressing gD receptors. HSV-1 entry into cells was determined by using β-galactosidase expressing HSV-1 reporter virus (gL86). As shown in FIG. 12A-C, gD-receptor (3-0ST-3, HVEM and nectin-1) expressing CHO-K1 cells preincubated with eN significantly blocked viral emry in a dose-dependent manner. The blocking activity of CN was pronounced at micromolar concentrations.

### EXAMPLE 9

CN Significantly Inhibits HSV-1 Entry into Natural Target Cells

To confirm blocking activity of CN on HSY-I entry, HeLa and primary cultures of human CF were used. It has been shown that HeLa and human CF naturally express gD receptors. As shown in FIG. 13A and 13B, the cells pre-incubated with CN showed significant blocking of HSV-1 entry in both HeLa and CF while corresponding untreated HeLa and CF were infected by the virus (black bars). Notably, strong inhibition of entry was observed at a lower concentration (<1 μM) of CN compared with our previous experiments using gD-receptor expressing CHO-K1 cells (FIG. 12). This raises the possibility that the effect of CN can be more pronounced in the natural target cells and that it may not depend on gD receptors. Taken together, the results indicated that the role of CN in HSV-1 entry blocking can vary between non-natural and natural target cells and the latter are strongly inhibited by CN. The inhibitory effect is likely dependent on the type of integrins expressed on the surface of cells.

### EXAMPLE 10

### Anti-HSV-1 Entry Inhibiting Activity of CN is not Viral Strain-Specific

The next question was to evaluate the broader significance of CN as an anti-HSV agent. Therefore the ability of CN to block viral entry in different clinical virulent strains of HSV-1 (F, G and MP) was tested. Here, nectin-1 expressing CHO Ig8 cells that express β-galactosidase upon viral entry were used. The cells were preincubated with CN at 10 μM and then infected with clinical isolates of HSV-1. Results from this experiment again showed that CN blocked the entry of different strains of HSV-1 as evident by ONPG assay (FIG. 14A).

### EXAMPLE 11

### CN Inhibits HSV-1 Plaque Formation

The ability of CN to inhibit HSV-1 spread was then tested. Viral plaque assay was conducted in the presence (FIG. 14B, panel i) and absence (FIG. 14B, panel ii) of CN. The cells were pre-incubated with CN at 10 μM and then infected with HSV-1. Results from this experiment showed that CN blocked the plaque formation as evident by plaque assay.

### EXAMPLE 12

### CN Treatment Inhibits HSV-1 Glycoprotein-Mediated Cell-to-Cell Fusion and Polykaryocyte Formation

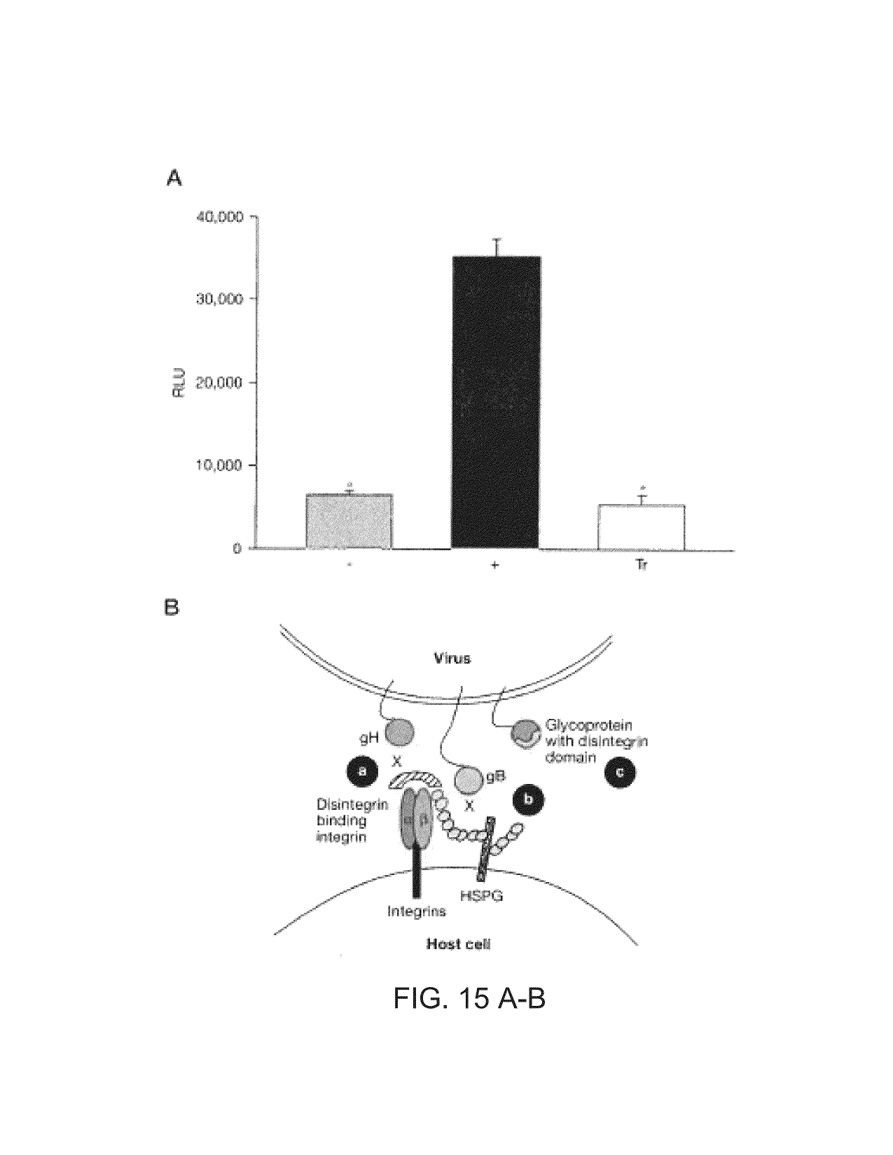

The role of CN during HSV-1 glycoprotein-mediated cell-to-cell fusion was then tested. Cell-to-cell fusion has been studied to demonstrate the viral and cellular requirements during virus-cell interactions and also as a means of viral spread. We sought to determine whether CN interaction with cellular integrins essential for viral entry affects cell-to-cell fusion. Surprisingly, natural target human CF treated with CN at 1 μM concentration impaired cell-to-cell fusion with the effector cells expressing HSV-1 glycoproteins (FIG. 15A; white bar). In parallel, the control CN-untreated target cells fused with effector cells showed high Luciferase readout (black bar), while the effector cells devoid of HSV-1 envelop glycoproteins failed to fuse (FIG. 15A; grey bar). The same response was observed when polykaryocyte formation was estimated (data not shown). The CN-treated target cells failed to form polykaryons when co-cultured with effector cells, while in the control untreated target cells larger polykaryons were observed.

The data described herein shows that a snake venom disinregrin broadly affects both HSV-1 (gL86) entry and spread in cell culture models. The anti-HSV-1 activity of CN was not limited to a particular receptor. Our results showed that HSV-1 entry was significantly blocked in CHO-K1 cells expressing either a sugar receptor (3-0ST-3 modified 30S HS) or a protein receptor (HVEM and nectin-1; FIG. 12). Similar blocking was also observed in natural target cells, specifically HeLa and human CF cells isolated from human cornea, which express nectin-1 and 3OST-3 receptor, respectively (FIG. 13). Interestingly, CN-mediated blocking was more pronounced even at lower concentrations (<1 μM) in naturally susceptible cells as compared to CHO-K1 cells expressing gD-receptors. This may be due to the fact that integrins are cell surface glycoproteins made of α and β subunits. The CHO-K1 cells we used in the experiments described herein express the endogenous αV unit, but lack the β3 subunit, while HeLa and CF express both integrin subunits. Therefore, it is possible that CN binding is determined by which β-integrin subunit is expressed. It is possible that the natural target cells have higher levels of the β-integrin subunit to which CN binds. We propose that CN might interfere with multiple critical steps involved in HSV-1 entry (FIG. 15B). For instance, CN-binding to cell surface integrins may affect HSV-1 glycoprotein H (gH) binding to integrins, or similarly, CN-interactions with integrins may also interfere with HSV-1 glycoprotein B (gB) binding to cell surface heparan sulfate since integrins and HSPG are expressed in close proximity. It is also possible that CN functions as a competitive inhibitor that competes with disintegrins expressed on HSV-1 envelope glycoproteins for integrin binding. It is also documented that integrin ligation by microbial pathogens, including viruses, elicits potent signalling responses that promote cytoskeletal reorganization and actin remodelling for viral inrernalization. One such function is integrin-dependent phagocytosis, a process that several inregrins are capable of mediating and that allows viral uptake via a novel phagocytotic mechanism. Recent work shows that cells expressing HSV-1 envelope glycoproteins can be taken up by a phagocytotic mechanism by human CFs, which is very similar to viral phagocytosis, and both processes involve actin remodelling. Interestingly, studies by the Applicants with cancer and endothelial cells show significant actin cytoskeleton disruption by CN and vicrostatin, the recombinant version of CN. Integrin-mediated signaling can also affect the host immune response, which can be devastating to cells and facilitate disease development. Activation of downstream molecules (PI3K, Rho family of GTPases, FAK) by integrins not only enhanced viral infection, but also contributed to the activation of proinflammatory cyrokines. Therefore, a class of proteins known as disinregrins, which were originally purified from snake venom and block inregrin function, could be very useful viral emry inhibitors as they not only block viral infection but inregrin-mediated immune response as well. Immune-mediated response, as a result of herpes virus infection is considered to be major cause of corneal blindness.

Molecules targeted to cel surface integrins, that lead to interference of the initial virus contact or recognition of cellular integrins, can be developed as antiviral candidates against diverse viral pathogens including herpesvirus infections for the following reasons. First, integrins are prime examples of physiologically important receptors that have been usurped by non-enveloped and enveloped viruses for attachment and/or cdl entry. In recent years, cellular integrins have emerged as attachment or “post-attachment” (internalization) receptors for a large number of viruses, including rhe HCVM, KSHV and Epstein-Barr virus. Second, integrin ligation by microbial pathogens, including viruses, elicits potent signalling responses that promote cytoskeletal reorganization/actin remodeling for viral internalization. Third, integrin-mediated signaling also affects host immune response, which alone can be devastating to cells and enhance disease development. It is also possible that CN may somehow interfere with HSV-1 gene-expression. Fourth, integrins remain attractive drug targets because of their ability to interfere with cell proliferation, migration, and/or tissue localization of inflammatory, angiogenic and tumour cells. Integrin-targeted drugs might also modulate virus-ligand affinity and signalling, a situation that could prove useful in controlling infectious diseases. The disintegrin being utilized here has major advantages. CN is natural in origin, but recombinant versions can be easily and consistently produced in a bacterial expression system with high yields, and, thus, are cost-efficient. In addition, structural variations introduced into recombinant disintegrins can fine-tune antiviral and cell survival activities. The development of a disintegrin rhat prevents the first step of viral attachment/fusion is likely to affect cell-signalling, viral replication and associated immune response, along with viral spread for many different viruses, irrespective of the viral genome and viral life cycle. Because of the ability to produce and modify disintegrin easily in the laboratory with low cytoxicity on host cell, the technology described herein is also likely to open a new door for inexpensive drug development with high potency and safety in their use against many other viruses including HCMV—a herpes virus that significantly depends on integrins for entry. Integrins also remain attractive drug targets for interfering with proliferation and migration of inflammatory cells, angiogenic endothelial cells and tumour cells. Integrin-targeted drugs might also modulate virus-ligand affinity and signalling, an application that could prove useful in controlling infections diseases.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

U.S. Publication No. 20060246541 by Minea et al., filed Feb. 9, 2006, and titled “Method of expressing proteins with disulfide bridges,” and PCT Patent Application No. PCT/US09/64256, filed Nov. 12, 2009, and titled “Method of expressing proteins with disulfide bridges with enhanced yields and activity,” and U.S. Provisional Patent Application No. 61/303,631, filed Feb. 11, 2010, and titled “Modified ADAM Disintegrin Domain Polypeptides and Uses Thereof” are related to this disclosure. The contents of all are incorporated herein by reference thereto including all figures.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

### CITED REFERENCES

1. Farooq, A. V., Valyi-Nagy, T., & Shukla D. (2010). Mediators and mechanisms of herpes simplex virus entry into ocular cells. Current Eye Res (In Press).
2. Liesegang, T. J., Melton, L. J., Daly, P. J. & Ilstrup. D. M. (1989). Epidemiology of ocular herpes simplex. Incidence in Rochester, Minn., 1950 through 1982. Arch Ophthalmol 107, 1155-1159.
3. Liesegang, T. J. (2001). Herpes simplex virus epidemiology and ocular importance. Cornea 20, 1-13.
4. Roizman, B., & Sears, A. E. (1996). Herpes simplex viruses and their replication. In. Fields, B. N., Knipe, D. M., Chanock, R. M., Hirsch, M. S., Melnick, J. L., Monath, T. P., Roizman, B. (Eds.), Virology, vol 2, 3^{rd} ed. Lippincott-Raven Publishers, Philadelphia, pp. 2231-2295.
5. Whitley, R. J., Kimberlin, D. W., & B. Roizman. (1998). Herpes simplex viruses. Clin Infect Dis 26, 541-553.
6. Whitley, R. J., & D. W. Kimberlin. (1997). Herpes simplex viruses. Clin Infect Dis 26, 97-109.
7. Shukla, D. & Spear, P. G. (2001). Herpesviruses and heparan sulfate: an intimate relationship in aid of viral entry. J Clin Invest 108, 503-510.
8. Spear, P. G., Eisenberg, R.J. & Cohen, G. H. (2000). Three classes of cell surface receptors for alphaherpesvirus entry. Virology 275, 1-8.
9. Browne, H., Bruun, B. & Minson, T. (2001). Plasma membrane requirements for cell fusion induced by herpes simplex virus type 1 glycoproteins gB, gD, gH and gL. J Gen Virol 82, 1419-1422.
10. Allison-T. T., Montgomery, R. I., Warner, M. S., Greaghty, R. J. & Spear, P. G. (2001). Contribution of gD receptors and glycosaminoglycan sulfation to cell fusion mediated by herpes simplex virus 1. Virus Res 74, 39-45.
11. Tiwari, V., Clement, C., Duncan, M. B., Chen, J., Liu, J. & Shukla, D. (2004). A role for 3-O-sulfated heparan in cell fusion induced by herpes simplex virus type 1 J Gen Virol 85, 805-809.
12. Montgomery, R. I., Warner, M. S., Lum, B. J. & Spear, P. G. (1996). Herpes simplex virus-1 entry into cells mediated by a novel member of the TNF/NGF receptor family. Cell 87, 427-436.
13. Cocchi, F., Menotti, L., Mirandola, P., Lopez, M. & Campadelli-Fiume, G. (1998). The Ectodomain of a Novel Member of the Immunoglobulin Subfamily Related to the Poliovirus Receptor Has the Attributes of a Bona Fide Receptor for Herpes Simplex Virus Types 1 and 2 in Human Cells. J Virol 72, 9992-10002.
14. Shukla, D., Liu, J., Blaiklock, P., Shworak, N. W., Bai, X., Esko, J. D., Cohen, G. H., Eisenberg, R. J., Rosenberg, R. D. & Spear, P. G. (1999). A novel role for 3-O-sulfated heparan sulfate in herpes simplex virus 1 entry. Cell 99, 13-22.
15. Shukla, S. Y., Singh, Y. K., & Shukla, D. (2009). Role of nectin-1, HVEM, and PILR-alpha in HSV-2 entry into human retinal pigment epithelial cells. Invest Ophthalmol Vis Sci. 50, 2878-2887.
16. Brantsaeter, A. B., K. Liestol, A. K. Goplen, O. Dunlop, and J. N. Bruun. 2002. CMV disease in AIDS patients: incidence of CMV disease and relation to survival in a population-based study from Oslo. Scand. J. Infect. Dis. 34:50-55
17. Ljungman, P. 1996. Cytomegalovirus infections in transplant patients. Scand. J. Infect. Dis. Suppl. 100:59-63.
18. Ramsay, M. E., E. Miller, and C. S. Peckham. 1991. Outcome of confirmed symptomatic congenital cytomegalovirus infection. Arch. Dis. Child. 66:1068-1069
19. Walmsley, S., and A. Tseng. 1999. Comparative tolerability of therapies for cytomegalovirus retinitis. Drug Saf. 21:203-224
20. Cinque, P., R. Marenzi, and D. Ceresa. 1997. Cytomegalovirus infections of the nervous system. Intervirology 40:85-97
21. Dankner, W. M., J. A. McCutchan, D. D. Richman, K. Hirata, and S. A. Spector. 1990. Localization of human cytomegalovirus in peripheral blood leukocytes by in situ hybridization. J. Infect. Dis. 161:31-36
22. Sinzger, C., and G. Jahn. 1996. Human cytomegalovirus cell tropism and pathogenesis. Intervirology 39:302-319
23. Sweet, C. 1999. The pathogenicity of cytomegalovirus. FEMS Microbiol. Rev. 23:457-482
24. van Den Pol, A. N., E. Mocarski, N. Saederup, J. Vieira, and T. J. Meier. 1999. Cytomegalovirus cell tropism, replication, and gene transfer in brain. J. Neurosci. 19:10948-10965
25. Ibanez, C. E., R. Schrier, P. Ghazal, C. Wiley, and J. A. Nelson. 1991. Human cytomegalovirus productively infects primary differentiated macrophages. J. Virol. 65:6581-6588
26. Nowlin, D. M., N. R. Cooper, and T. Compton. 1991. Expression of a human cytomegalovirus receptor correlates with infectibility of cells. J. Virol. 65:3114-3121
27. Sinzger, C., M. Kahl, K. Laib, K. Klingel, P. Rieger, B. Plachter, and G. Jahn. 2000. Tropism of human cytomegalovirus for endothelial cells is determined by a post-entry step dependent on efficient translocation to the nucleus. J. Gen. Virol. 81:3021-3035
28. Hobom, U., W. Brune, M. Messerle, G. Hahn, and U. H. Koszinowski. 2000. Fast screening procedures for random transposon libraries of cloned herpesvirus genomes: mutational analysis of human cytomegalovirus envelope glycoprotein genes. J. Virol. 74:7720-7729
29. Spear, P. G., and R. Longnecker. 2003. Herpesvirus entry: an update. J. Virol. 77:10179-10185
30. Boehme, K. W., J. Singh, S. T. Perry, and T. Compton. 2004. Human cytomegalovirus elicits a coordinated cellular antiviral response via envelope glycoprotein B. J. Virol. 78:1202-1211.
31. Boyle, K. A., and T. Compton. 1998. Receptor-binding properties of a soluble form of human cytomegalovirus glycoprotein B. J. Virol. 72:1826-1833
32. Boyle, K. A., R. L. Pietropaolo, and T. Compton. 1999. Engagement of the cellular receptor for glycoprotein B of human cytomegalovirus activates the interferon-responsive pathway. Mol. Cell. Biol. 19:3607-3613
33. Compton, T., D. M. Nowlin, and N. R. Cooper. 1993. Initiation of human cytomegalovirus infection requires initial interaction with cell surface heparan sulfate. Virology 193:834-841
34. Kari, B., and R. Gehrz. 1992. A human cytomegalovirus glycoprotein complex designated gC-II is a major heparin-binding component of the envelope. J. Virol. 66:1761-1764.[Abstract/Free Full Text]
35. Kari, B., and R. Gehrz. 1993. Structure, composition and heparin binding properties of a human cytomegalovirus glycoprotein complex designated gC-II. J. Gen. Virol. 74:255-264
36. Zhou, Q, et al., Molecular cloning and functional expression of contortrostatin, a homodimeric disintegrin from southern copperhead snake venom., Arch Biochem Biophys 2000. 375(2): 278-88.
37. Dean, H. J., Terhune, S., Shieh, M. T., Susmarski, N., Spear, P. G., 1994. Single amino acid substitutions in gD of herpes simplex virus 1 confer resistant to gD-mediated interference and cause cell type-dependent alterations in infectivity. Virology 199, 67-80.
38. Trikha, M., et al., Purification and characterization of platelet aggregation inhibitors from snake venoms. Thromb Res, 1994. 73(1): 39-52.
39. Bilgrami, S., et al., Crystal structure of schistatin, a disintegrin homodimer from saw-scaled viper (Echis carinatus) at 2.5 A resolution. J Mol Biol, 2004. 341(3): 829-37.
40. Lowry, O. H., et al., Protein measurement with the Folin phenol reagent. J Biol Chem., 1951. Nov;193(1):265-75.
41. Hartree E. F., Determination of protein: a modification of the Lowry method that gives a linear photometric response. Anal Biochem., 1972 Aug;48(2):422-7.
42. Edwards, D. R., M. M. Handsley, and C. J. Pennington, The ADAM metalloproteinases. Mol Aspects Med, 2008. 29(5): p. 258-89.
43. Mochizuki, S. and Y. Okada, ADAMs in cancer cell proliferation and progression. Cancer Sci, 2007. 98(5): p. 621-8.
44. Reiss, K., A. Ludwig, and P. Saftig, Breaking up the tie: disintegrin-like metalloproteinases as regulators of cell migration in inflammation and invasion. Pharmacol Ther, 2006. 111(3): p. 985-1006.
45. Tousseyn, T., et al., (Make) stick and cut loose—disintegrin metalloproteases in development and disease. Birth Defects Res C Embryo Today, 2006. 78(1): p. 24-46.
46. Arribas, J., J. J. Bech-Serra, and B. Santiago-Josefat, ADAMs, cell migration and cancer. Cancer Metastasis Rev, 2006. 25(1): p. 57-68.
47. Duffy M. J. et al., Role of ADAMs in cancer formation and progression. Clin Cancer Res. 2009 Feb 15;15(4):1140-4.
48. LaVallie, E. R., et al., A thioredoxin gene fusion expression system that circumvents inclusion body formation in the E. coli cytoplasm. Biotechnology (NY), 1993. 11(2): p. 187-93.
49. Bessette et al., Efficient folding of proteins with multiple disulfide bonds in the Escherichia coli cytoplasm. Proc. Natl. Acad. Sci (USA) 1999, 96(24):13703-8.
50. Prinz, W. A., et al., The role of the thioredoxin and glutaredoxin pathways in reducing protein disulfide bonds in the Escherichia coli cytoplasm. J Biol Chem, 1997. 272(25): p. 15661-7.
51. Swenson, S., et al., Intravenous liposomal delivery of the snake venom disintegrin contortrostatin limits breast cancer progression. Mol Cancer Ther, 2004. 3(4): p. 499-511.
52. Minea, R., et al., Development of a novel recombinant disintegrin, contortrostatin, as an effective anti-tumor and antiangiogenic agent. Pathophysiol Haemost Thromb, 2005. 34(4-5): p. 177-83.
53. Coulson et al., Rotavirus contains integrin ligand sequences and a disintegrin-like domain that are implicated in virus entry into cells. Proc. Natl. Acad. Sci. USA 1997, 94:5389-5394.
54. Feire et al., Cellular integrins function as entry receptors for human cytomegalovirus via a highly conserved disintegrin-like domain. Proc. Natl. Acad. Sci. USA 2004, 101:15470-15475.
55. Stewart P L, Nemerow G R. Cell integrins: commonly used receptors for diverse viral pathogens. Trends Microbiol. 2007 Nov;15(11):500-7. Epub 2007 Nov 7.
56. Dorner M, Zucol F, Alessi D, Haerle S K, Bossart W, Weber M, Byland R, Bernasconi M, Berger C, Tugizov S, Speck R F, Nadal D. beta1 integrin expression increases susceptibility of memory B cells to Epstein-Barr virus infection. J Virol. 2010 Jul;84(13):6667-77. Epub 2010 Apr 28.
57. Cheng J, Ke Q, Jin Z, et al. (May 2009). “Cytomegalovirus infection causes an increase of arterial blood pressure”. PLoS Pathog. 5 (5).
58. Abban C Y, Meneses P I. (2010). Usage of heparan sulfate, integrins, and FAK in HPV16 infection. Virology. 2010 Jul 20;403(1):1-16. Epub 2010 May 2.
59. Cseke G, Maginnis M S, Cox R G, Tollefson S J, Podsiad A B, Wright D W, Dermody T S, Williams J V. Integrin alphavbeta1 promotes infection by human metapneumovirus. Proc Natl Acad Sci USA. 2009 Feb 3;106(5):1566-71.
60. Schaack et al., J. Virol. 69:3920-3923 (1995).

What is claimed is:

1. A method of inhibiting viral infection of a host cell comprising contacting the host cell with an effective amount of a polypeptide comprising a disintegrin domain.

2. The method of claim 1 wherein said polypeptide is CN, VCN or a MAP.

3. The method of claim 2 wherein said polypeptide comprising a disintegrin domain comprises a fusion protein.

4. The method of claim 3 wherein said fusion comprises thioredoxin A or fragment thereof.

5. The method of claim 2 wherein said MAP is one or more of MAP1, MAP2, MAP3, MAP6, MAP7, MAP8, MAP9, MAP10, MAP11, MAP12, MAP15, MAP17, MAP18, MAP19, MAP20, MAP21, MAP22, MAP23, MAP28, MAP29, MAP30, MAP32 or MAP33.

6. The method of claim 1 wherein said host cell is selected from the group consisting of epithelial cell, fibroblast, endothelial cell, smooth muscle cell, stromal cell, monocyte, macrophage, neutrophil, neuronal cell, and hepatocyte.

7. The method of claim 6 wherein said epithelial cell is selected from the group consisting of skin epithelial cell, corneal epithelial cell, and retinal pigment epithelial cell.

8. The method of claim 1 wherein the viral infection is inhibited by reducing at least one of the stages of infection selected from the group consisting of viral entry, signaling, internalization, and transport.

9. The method of claim 1 wherein said virus is selected from the group consisting of Adenovirus, Herpesvirus, Human Papilloma Virus (HPV), Human metapneumovirus, Hantavirus, Picornovirus, Rotavirus, West Nile virus, foot-and-mouth disease virus, and ebola virus.

10. The method of claim 1 wherein the concentration of said polypeptide is at least 0.085 nM.

11. The method of claim 10 wherein the concentration of said disintegrin is at least 125 nM.

12. The method of claim 11 wherein the concentration of said disintegrin is at least 200 nM.

13. The method of claim 1 wherein said polypeptide is administered as a pharmaceutical composition.

14. The method of claim 1 further comprising administering an effective amount of an antiviral drug or vaccine.

15. The method of claim 14 wherein said antiviral drug is selected from the group consisting of Rifampin, Ribavirin, Pleconaryl, Cidofovir, Acyclovir, Pencyclovir, Gancyclovir, Valacyclovir, Famciclovir, Foscarnet, Vidarabine, Amantadine, Zanamivir, Oseltamivir, Resquimod, antiprotease, pegylated interferon, lopinivir, saquinivir, amprenavir, HIV fusion inhibitors, AZT, Lamivudine, Abacavir, non-nucleotide HIV RT inhibitors, Doconosol, Interferons, Butylated Hydroxytoluene (BHT) and Hypericin.

16. A method for treating or preventing viral infection of a subject in need thereof comprising administering to said subject a therapeutically effective amount of one or more polypeptides comprising a disintegrin domain.

17. The method of claim 16 wherein said polypeptide comprising a disintegrin domain is CN, VCN or a MAP.

18. The method of claim 17 wherein said disintegrin comprises a fusion protein.

19. The method of claim 18 wherein said fusion comprises thioredoxin A or fragment thereof.

20. The method of claim 17 wherein said MAP is one or more of MAP1, MAP2, MAP3, MAP6, MAP7, MAPS, MAP9, MAP10, MAP11, MAP12, MAP15, MAP17, MAP18, MAP19, MAP20, MAP21, MAP22, MAP23, MAP28, MAP29, MAP30, MAP32 or MAP33.

21. The method of claim 16 wherein said host cell is selected from the group consisting of epithelial cell, fibroblast, endothelial cell, smooth muscle cell, stromal cell, monocyte, macrophage, neutrophil, neuronal cell, and hepatocyte.

22. The method of claim 21 wherein said epithelial cell is selected from the group consisting of skin epithelial cell, corneal epithelial cell, and retinal pigment epithelial cell.

23. The method of claim 16 wherein the viral infection is inhibited by reducing at least one of the stages of infection selected from the group consisting of viral entry, signaling, internalization, and transport.

24. The method of claim 16 wherein said virus is selected from the group consisting of Adenovirus, Herpesvirus, Human Papilloma Virus (HPV), Human metapneumovirus, Hantavirus, Picornovirus, Rotavirus, West Nile virus, foot-and-mouth disease virus, and ebola virus.

25. The method of claim 16 wherein the therapeutically effective amount of said disintegrin is at least 0.1 mg/kg.

26. The method of claim 25 wherein the therapeutically effective amount of said disintegrin is at least 1 mg/kg.

27. The method of claim 26 wherein the therapeutically effective amount of said disintegrin is at least 10 mg/kg.

28. The method of claim 16 wherein said polypeptide is administered as a pharmaceutical composition.

29. The method of claim 16 further comprising administering to the subject an effective amount of an antiviral drug or vaccine.

30. The method of claim 29 wherein said antiviral drug is selected from the group consisting of Rifampin, Ribavirin, Pleconaryl, Cidofovir, Acyclovir, Pencyclovir, Gancyclovir, Valacyclovir, Famciclovir, Foscarnet, Vidarabine, Amantadine, Zanamivir, Oseltamivir, Resquimod, antiprotease, pegylated interferon, lopinivir, saquinivir, amprenavir, HIV fusion inhibitors, AZT, Lamivudine, Abacavir, non-nucleotide HIV RT inhibitors, Doconosol, Interferons, Butylated Hydroxytoluene (BHT) and Hypericin.

31. A method for screening a polypeptide comprising a disintegrin domain for prophylactic or therapeutic antiviral activity comprising contacting a host cell with (a) a candidate polypeptide and (b) a candidate virus, in either order, and determining if said disintegrin inhibits infection.

32. The method of claim 31 wherein said polypeptide is CN, VCN or a MAP.

33. The method of claim 32 wherein said polypeptide comprising a disintegrin domain comprises a fusion protein.

34. The method of claim 33 wherein said fusion comprises thioredoxin A or fragment thereof.

35. The method of claim 32 wherein said MAP is selected from one or more of MAP1, MAP2, MAP3, MAP6, MAP7, MAP8, MAP9, MAP10, MAP11, MAP12, MAP15, MAP17, MAP18, MAP19, MAP20, MAP21, MAP22, MAP23, MAP28, MAP29, MAP30, MAP32 or MAP33.

36. The method of claim 31 wherein said host cell is selected from the group consisting of epithelial cell, fibroblast, endothelial cell, smooth muscle cell, stromal cell, monocyte, macrophage, neutrophil, neuronal cell, and hepatocyte.

37. The method of claim 36 wherein said epithelial cell is selected from the group consisting of skin epithelial cell, corneal epithelial cell, and retinal pigment epithelial cell.

38. The method of claim 31 wherein the viral infection is inhibited by reducing at least one of the stages of infection selected from the group consisting of viral entry, signaling, internalization, and transport.

39. The method of claim 31 wherein said virus is selected from the group consisting of Adenovirus, Herpesvirus, Human Papilloma Virus (HPV), Human metapneumovirus, Hantavirus, Picornovirus, Rotavirus, West Nile virus, foot-and-mouth disease virus, and ebola virus.

40. A kit for one or more of: inhibiting viral entry into a host cell, treating or preventing a viral infection in a subject in need of such treatment, or screening a polypeptide having a disintegrin domain for prophylactic or therapeutic antiviral activity, the kit comprising one or more a polypeptide comprising a disintegrin domain that prevents or treats viral infection in a host cell or subject.

41. The kit of claim 40 further comprising instructions for the intended use.


SEQUENCE LISTING (104 sequences)

SEQ ID NO: 1 - protein, 483 aa, Agkistrodon contortrix
MIQVLLVTLCLAAFPYQGSSIILESGNVNDYEVLYPQKVTALPKGAVQPKYEDTMQYEFKVNGEPVVLHLEKNKGLFSKDYSETHYSSDGRKITTNPPVEDHCYYHGRIQNDADSTASISACNGLKGHFKLQGETYLIEPLKLSDSEAHAVYKYENVEKEDEAPKMCGVTQTNWESDEPIKKASQLNLTPEQQGFPQRYIELVVVADHRMFTKYNGNLNTIRIWVHELVNTMNVFYRPLNIRVSLTDLEVWSDQDLINVQPAAADTLEAFGDWRETVLLNRISHDNAQLLTAIELDGETIGLANRGTMCDPKLSTGIVQDHSAINLWVAVTMAHEMGHNLGISHDGNQCHCDANSCIMSEELREQLSFEFSDCSQNQYQTYLTDHNPQCMLNEPLRTDIVSTPVSGNELLETGEESDFDAPANPCCDAATCKLTTGSQCADGLCCDQCKFMKEGTVCRRARGDDLDDYCNGISAGCPRNPFHA

SEQ ID NO: 2 - protein, 6 aa, Echis cannatus
HGKPAT

SEQ ID NO: 3 - protein, 7 aa, Artificial Sequence
  Description of Artificial Sequence Synthetic peptide
ENLYFQG

SEQ ID NO: 4 - protein, 65 aa, Agkistrodon contortrix
DAPANPCCDAATCKLTTGSQCADGLCCDQCKFMKEGTVCRRARGDDLDDYCNGISAGCPRNPFHA

SEQ ID NO: 5 - protein, 68 aa, Artificial Sequence
  Description of Artificial Sequence Synthetic polypeptide
DAPANPCCDAATCKLTTGSQCADGLCCDQCKFMKEGTVCRRARGDDLDDYCNGISAGCPRNPHKGPAT

SEQ ID NO: 6 - DNA, 59 bp, Artificial Sequence
  Description of Artificial Sequence Synthetic primer
gttccagatctcgagaatctttacttccaaggagacgctcctgcaaatccgtgctgcga

SEQ ID NO: 7 - DNA, 51 bp, Artificial Sequence
  Description of Artificial Sequence Synthetic primer
gttattcgccatggcttaggcatggaagggatttctgggacagccagcaga

SEQ ID NO: 8 - DNA, 66 bp, Artificial Sequence
  Description of Artificial Sequence Synthetic primer
gttattcgccatggcttaagtagctggacccttgtggggatttctgggacagccagcagatatgcc

SEQ ID NO: 9 - protein, 78 aa, Unknown
  Description of Unknown disintegrin domain of PIII-class snake venom metalloprotease (VAP1) polypeptide
GEECDCGSPRTCRDPCCDATTCKLRQGAQCAEGLCCDQCRFKGAGTECRAAKDECDMADVCTGRSAECTDRFQRNGQP

SEQ ID NO: 10 - protein, 78 aa, Unknown
  Description of Unknown disintegrin domain of PIII-class snake venom metalloprotease (catrocollastatin) polypeptide
GEECDCGTPENCQNECCDAATCKLKSGSQCGHGDCCEQCKFSKSGTECRASMSECDPAEHCTGQSSECPADVFHKNGQ

SEQ ID NO: 11 - protein, 83 aa, Unknown
  Description of Unknown long-sized snake venom disintegrin domain (salmosin3) polypeptide
SPPVCGNYYPEVGEDCDCGPPANCQNPCCDAATCGLTTGSQCAEGLCCDQCRLKKAGTICRKARGDNPDDRCTGQSGVCPRNT

SEQ ID NO: 12 - protein, 83 aa, Bitis arietans
SPPVCGNKILEEGEECDCGSPANCQDRCCNAATCKLTPGSQCNHGECCDQCKFKKAGTVCRIARGDWNDDYCTGKSSDCPWNH

SEQ ID NO: 13 - protein, 70 aa, Unknown
  Description of Unknown prototypical medium-sized snake venom disintegrin (trimestatin) polypeptide
GEECDCGSPSNPCCDAATCKLRPGAQCADGLCCDQCRFKKKRTICRIARGDFPDDRCTGQSADCPRWNDL

SEQ ID NO: 14 - protein, 78 aa, Homo sapiens
GEECDCGPAQECTNPCCDAHTCVLKPGFTCAEGECCESCQIKKAGSICRPAKDECDFPEMCTGHSPACPKDQFRVNGF

SEQ ID NO: 15 - protein, 78 aa, Homo sapiens
GEQCDCGPPEDCRNRCCNSTTCQLAEGAQCAHGTCCQECKVKPAGELCRPKKDMCDLEEFCDGRHPECPEDAFQENGT

SEQ ID NO: 16 - protein, 78 aa, Homo sapiens
GEECDCGEPEECMNRCCNATTCTLKPDAVCAHGLCCEDCQLKPAGTACRDSSNSCDLPEFCTGASPHCPANVYLHDGH

SEQ ID NO: 17 - protein, 78 aa, Homo sapiens
GEECDCGEEEECNNPCCNASNCTLRPGAECAHGSCCHQCKLLAPGTLCREQARQCDLPEFCTGKSPHCPTNFYQMDGT

SEQ ID NO: 18 - protein, 78 aa, Homo sapiens
GEDCDCGTSEECTNICCDAKTCKIKATFQCALGECCEKCQFKKAGMVCRPAKDECDLPEMCNGKSGNCPDDRFQVNGF

SEQ ID NO: 19 - protein, 78 aa, Homo sapiens
GEECDCGPGQECRDLCCFAHNCSLRPGAQCAHGDCCVRCLLKPAGALCRQAMGDCDLPEFCTGTSSHCPPDVYLLDGS

SEQ ID NO: 20 - protein, 76 aa, Homo sapiens
TEQCDCGSLCQHHACCDENCILKAKAECSDGPCCHKCKFHRKGYPCCPSSRSCDLPEFCNGTSALCPNNRHKQDGS

SEQ ID NO: 21 - protein, 81 aa, Homo sapiens
GEECDCGTEQDCALIGETCCDIATCRFKAGSNCAEGPCCENCLFMSKERMCRPSFEECDLPEYCNGSSASCPENHYVQTGH

SEQ ID NO: 22 - protein, 81 aa, Homo sapiens
TEQCDCGYKEACTHKKCCNPADCTLVRSAECGTGSCCNNKTCTIHERGHVCRKSVDMCDFPEYCNGTSEFCVPDVKAADLE

SEQ ID NO: 23 - protein, 78 aa, Homo sapiens
REECDCGSFKQCYASYCCQSDCHLTPGSICHIGECCTNCSFSPPGTLCRPIQNICDLPEYCHGTTVTCPANVYMQDGT

SEQ ID NO: 24 - protein, 78 aa, Homo sapiens; identical to SEQ ID NO: 14

SEQ ID NO: 25 - protein, 78 aa, Homo sapiens; identical to SEQ ID NO: 15

SEQ ID NO: 26 - protein, 79 aa, Homo sapiens
GEECDCGTPKECELDPCCEGSTCKLKSFAECAYGDCCKDCRFLPGGTLCRGKTSECDVPEYCNGSSQFCQPDVFIQNGY

SEQ ID NO: 27 - protein, 86 aa, Homo sapiens
GEECDCGYSDQCKDECCFDANQPEGRKCKLKPGKQCSPSQGPCCTAQCAFKSKSEKCRDDSDCAREGICNGFTALCPASDPKPNFT

SEQ ID NO: 28 - protein, 79 aa, Homo sapiens
GEECDCGSVQECSRAGGNCCKKCTLTHDAMCSDGLCCRRCKYEPRGVSCREAVNECDIAETCTGDSSQCPPNLHKLDGY

SEQ ID NO: 29 - protein, 78 aa, Homo sapiens; identical to SEQ ID NO: 16

SEQ ID NO: 30 - protein, 79 aa, Homo sapiens
GEQCDCGFLDDCVDPCCDSLTCQLRPGAQCASDGPCCQNCQLRPSGWQCRPTRGDCDLPEFCPGDSSQCPPDVSLGDGE

SEQ ID NO: 31 - protein, 80 aa, Homo sapiens
GEECDPGIMYLNNDTCCNSDCTLKEGVQCSDRNSPCCKNCQFETAQKKCQEAINATCKGVSYCTGNSSECPPPGNAEDDT

SEQ ID NO: 32 - protein, 81 aa, Homo sapiens
NEECDCGNKNECQFKKCCDYNTCKLKGSVKCGSGPCCTSKCELSIAGTPCRKSIDPECDFTEYCNGTSSNCVPDTYALNGR

SEQ ID NO: 33 - protein, 78 aa, Homo sapiens; identical to SEQ ID NO: 17

SEQ ID NO: 34 - protein, 78 aa, Homo sapiens
GEECDCGTIRQCAKDPCCLLNCTLHPGAACAFGICCKDCKFLPSGTLCRQQVGECDLPEWCNGTSHQCPDDVYVQDGI

SEQ ID NO: 35 - protein, 78 aa, Homo sapiens
EEQCDCGSVQQCEQDACCLLNCTLRPGAACAFGLCCKDCKFMPSGELCRQEVNECDLPEWCNGTSHQCPEDRYVQDGI

SEQ ID NO: 36 - protein, 79 aa, Homo sapiens
GEECDCGTPAECVLEGAECCKKCTLTQDSQCSDGLCCKKCKFQPMGTVCREAVNDCDIRETCSGNSSQCAPNIHKMDGY

SEQ ID NO: 37 - protein, 78 aa, Homo sapiens
GEECDCGFHVECYGLCCKKCSLSNGAHCSDGPCCNNTSCLFQPRGYECRDAVNECDITEYCTGDSGQCPPNLHKQDGY

SEQ ID NO: 38 - protein, 78 aa, Homo sapiens; identical to SEQ ID NO: 18

SEQ ID NO: 39 - protein, 78 aa, Homo sapiens
GEECDCGPLKHCAKDPCCLSNCTLTDGSTCAFGLCCKDCKFLPSGKVCRKEVNECDLPEWCNGTSHKCPDDFYVEDGI

SEQ ID NO: 40 - protein, 78 aa, Homo sapiens
NEECDCGSTEECQKDRCCQSNCKLQPGANCSIGLCCHDCRFRPSGYVCRQEGNECDLAEYCDGNSSSCPNDVYKQDGT

SEQ ID NO: 41 - protein, 80 aa, Homo sapiens
NEICDCGTEAQCGPASCCDFRTCVLKDGAKCYKGLCCKDCQILQSGVECRPKAHPECDIAENCNGSSPECGPDITLINGL

SEQ ID NO: 42 - protein, 78 aa, Homo sapiens; identical to SEQ ID NO: 19

SEQ ID NO: 43 - protein, 72 aa, Artificial Sequence
  Description of Artificial Sequence Synthetic polypeptide
TEQCDCGSLHACCDENCILKAKAECSDGPCCHKCKFHRKGYPCRPSSRSDLPEFCNGTSALCPNNRHKQDGS

SEQ ID NO: 44 - protein, 77 aa, Artificial Sequence
  Description of Artificial Sequence Synthetic polypeptide
GEECDCGTEQDIGETCCDIATCRFKAGSNCAEGPCCENCLFMSKERMCRPSFEEDLPEYCNGSSASCPENHYVQTGH

SEQ ID NO: 45 - protein, 77 aa, Artificial Sequence
  Description of Artificial Sequence Synthetic polypeptide
TEQCDCGYKEAKKCCNPADCTLVRSAECGTGSCCNNKTCTIHERGHVCRKSVDMDFPEYCNGTSEFCVPDVKAADLE

SEQ ID NO: 46 - protein, 74 aa, Artificial Sequence
  Description of Artificial Sequence Synthetic polypeptide
REECDCGSFKQSYCCQSDCHLTPGSICHIGECCTNCSFSPPGTLCRPIQNIDLPEYCHGTTVTCPANVYMQDGT

SEQ ID NO: 47 - protein, 74 aa, Artificial Sequence
  Description of Artificial Sequence Synthetic polypeptide
GEECDCGPAQEPCCDAHTCVLKPGFTCAEGECCESCQIKKAGSICRPAKDEDFPEMCTGHSPACPKDQFRVNGF

SEQ ID NO: 48 - protein, 74 aa, Artificial Sequence
  Description of Artificial Sequence Synthetic polypeptide
GEQCDCGPPEDRCCNSTTCQLAEGAQCAHGTCCQECKVKPAGELCRPKKDMDLEEFCDGRHPECPEDAFQENGT

SEQ ID NO: 49 - protein, 75 aa, Artificial Sequence
  Description of Artificial Sequence Synthetic polypeptide
GEECDCGTPKEDPCCEGSTCKLKSFAECAYGDCCKDCRFLPGGTLCRGKTSEDVPEYCNGSSQFCQPDVFIQNGY

SEQ ID NO: 50 - protein, 75 aa, Artificial Sequence
  Description of Artificial Sequence Synthetic polypeptide
GEECDCGSVQEAGGNCCKKCTLTHDAMCSDGLCCRRCKYEPRGVSCREAVNEDIAETCTGDSSQCPPNLHKLDGY

SEQ ID NO: 51 - protein, 74 aa, Artificial Sequence
  Description of Artificial Sequence Synthetic polypeptide
GEECDCGEPEERCCNATTCTLKPDAVCAHGLCCEDCQLKPAGTACRDSSNSDLPEFCTGASPHCPANVYLHDGH

SEQ ID NO: 52 - protein, 75 aa, Artificial Sequence
  Description of Artificial Sequence Synthetic polypeptide
GEQCDCGFLDDPCCDSLTCQLRPGAQCASDGPCCQNCQLRPSGWQCRPTRGDDLPEFCPGDSSQCPPDVSLGDGE

SEQ ID NO: 53 - protein, 74 aa, Artificial Sequence
  Description of Artificial Sequence Synthetic polypeptide
GEECDCGEEEEPCCNASNCTLRPGAECAHGSCCHQCKLLAPGTLCREQARQDLPEFCTGKSPHCPTNFYQMDGT

SEQ ID NO: 54 - protein, 74 aa, Artificial Sequence
  Description of Artificial Sequence Synthetic polypeptide
GEECDCGTIRQDPCCLLNCTLHPGAACAFGICCKDCKFLPSGTLCRQQVGEDLPEWCNGTSHQCPDDVYVQDGI

SEQ ID NO: 55 - protein, 74 aa, Artificial Sequence
  Description of Artificial Sequence Synthetic polypeptide
EEQCDCGSVQQDACCLLNCTLRPGAACAFGLCCKDCKFMPSGELCRQEVNEDLPEWCNGTSHQCPEDRYVQDGI

SEQ ID NO: 56 - protein, 75 aa, Artificial Sequence
  Description of Artificial Sequence Synthetic polypeptide
GEECDCGTPAEEGAECCKKCTLTQDSQCSDGLCCKKCKFQPMGTVCREAVNDDIRETCSGNSSQCAPNIHKMDGY

SEQ ID NO: 57 - protein, 74 aa, Artificial Sequence
  Description of Artificial Sequence Synthetic polypeptide
GEECDCGFHVELCCKKCSLSNGAHCSDGPCCNNTSCLFQPRGYECRDAVNEDITEYCTGDSGQCPPNLHKQDGY

SEQ ID NO: 58 - protein, 74 aa, Artificial Sequence
  Description of Artificial Sequence Synthetic polypeptide
GEDCDCGTSEEICCDAKTCKIKATFQCALGECCEKCQFKKAGMVCRPAKDEDLPEMCNGKSGNCPDDRFQVNGF

SEQ ID NO: 59 - protein, 74 aa, Artificial Sequence
  Description of Artificial Sequence Synthetic polypeptide
GEECDCGPLKHDPCCLSNCTLTDGSTCAFGLCCKDCKFLPSGKVCRKEVNEDLPEWCNGTSHKCPDDFYVEDGI

SEQ ID NO: 60 - protein, 74 aa, Artificial Sequence
  Description of Artificial Sequence Synthetic polypeptide
NEECDCGSTEEDRCCQSNCKLQPGANCSIGLCCHDCRFRPSGYVCRQEGNEDLAEYCDGNSSSCPNDVYKQDGT

SEQ ID NO: 61 - protein, 74 aa, Artificial Sequence
  Description of Artificial Sequence Synthetic polypeptide
GEECDCGPGQELCCFAHNCSLRPGAQCAHGDCCVRCLLKPAGALCRQAMGDDLPEFCTGTSSHCPPDVYLLDGS

SEQ ID NO: 62 - DNA, 216 bp, Artificial Sequence
  Description of Artificial Sequence Synthetic polynucleotide
acggagcagtgtgactgtggttctctacatgcatgctgtgatgaaaactgtatactgaaggcgaaagcagagtgcagtgatggtccatgttgtcataagtgtaaatttcaccgtaagggatatccttgcaggccttctagtcgttccgatctcccagaattttgcaatggtacatctgcattatgccccaacaacaggcataagcaagatggctca

SEQ ID NO: 63 - DNA, 231 bp, Artificial Sequence
  Description of Artificial Sequence Synthetic polynucleotide
ggagaggagtgtgactgtgggactgaacaggatattggagaaacatgctgtgatattgccacatgtagatttaaagccggttcaaactgtgctgaaggaccatgctgcgaaaactgtctatttatgtcaaaagaaagaatgtgtaggccttcctttgaagaagacctccctgaatattgcaatggatcatctgcatcatgcccagaaaaccactatgttcagactgggcat

SEQ ID NO: 64 - DNA, 231 bp, Artificial Sequence
  Description of Artificial Sequence Synthetic polynucleotide
acagagcagtgtgattgtggctataaagaggcaaaaaaatgctgtaatcctgcagattgtactctagttagatctgcagaatgtggcactggatcatgctgtaacaataaaacttgtacgatccacgaaagaggccatgtctgcagaaaaagtgtagatatggattttccagaatattgcaatggaacatctgagttttgtgtacctgatgtgaaagctgctgatttagaa

SEQ ID NO: 65 - DNA, 222 bp, Artificial Sequence
  Description of Artificial Sequence Synthetic polynucleotide
agggaggaatgtgactgtggctccttcaagcagagttattgctgccaaagtgactgtcacttaacaccggggagcatctgccatataggagagtgctgtacaaactgcagcttctccccaccagggactctctgcagacctatccaaaatatagaccttccagagtactgtcacgggaccaccgtgacatgtcccgcaaacgtttatatgcaagatggaacc

SEQ ID NO: 66 - DNA, 222 bp, Artificial Sequence
  Description of Artificial Sequence Synthetic polynucleotide
ggtgaagagtgtgactgtggccctgctcaggagccttgctgtgatgcacacacatgtgtactgaagccaggatttacttgtgcagaaggagaatgctgtgaatcttgtcagataaaaaaagcagggtccatatgcagaccggcgaaagatgaagattttcctgagatgtgcactggccactcgcctgcctgtcctaaggaccagttcagggtcaatggattt

SEQ ID NO: 67 - DNA, 222 bp, Artificial Sequence
  Description of Artificial Sequence Synthetic polynucleotide
ggggagcagtgcgactgcggcccccccgaggaccgctgctgcaactctaccacctgccagctggctgagggggcccagtgtgcgcacggtacctgctgccaggagtgcaaggtgaagccggctggtgagctgtgccgtcccaagaaggacatggacctcgaggagttctgtgacggccggcaccctgagtgcccggaagacgccttccaggagaacggcacg

SEQ ID NO: 68 - DNA, 225 bp, Artificial Sequence
  Description of Artificial Sequence Synthetic polynucleotide
ggggaagagtgtgactgtggtactccaaaggaagacccttgctgcgaaggaagtacctgtaagcttaaatcatttgctgagtgtgcatatggtgactgttgtaaagactgtcggttccttccaggaggtactttatgccgaggaaaaaccagtgaggatgttccagagtactgcaatggttcttctcagttctgtcagccagatgtttttattcagaatggatat

SEQ ID NO: 69 - DNA, 246 bp, Artificial Sequence
  Description of Artificial Sequence Synthetic polynucleotide
ggtgaagaatgtgattgtggctatagtgaccaggaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagacgcaagggaaggaatatgtaatggcttcacagctctctgcccagcatctgaccctaaaccaaacttcaca

SEQ ID NO: 70 - DNA, 225 bp, Artificial Sequence
  Description of Artificial Sequence Synthetic polynucleotide
ggggaggagtgcgactgcggctcggtgcaggaggcaggtggcaactgctgcaagaaatgcaccctgactcacgacgccatgtgcagcgacgggctctgctgtcgccgctgcaagtacgaaccacggggtgtgtcctgccgagaggccgtgaacgaggacatcgcggagacctgcaccggggactctagccagtgcccgcctaacctgcacaagctggacggttac

SEQ ID NO: 71 - DNA, 222 bp, Artificial Sequence
  Description of Artificial Sequence Synthetic polynucleotide
ggagaggagtgtgactgtggggagccagaggaacgctgctgcaatgccaccacctgtaccctgaagccggacgctgtgtgcgcacatgggctgtgctgtgaagactgccagctgaagcctgcaggaacagcgtgcagggactccagcaactccgacctcccagagttctgcacaggggccagccctcactgcccagccaacgtgtacctgcacgatgggcac

SEQ ID NO: 72 - DNA, 225 bp, Artificial Sequence
  Description of Artificial Sequence Synthetic polynucleotide
ggcgagcagtgtgactgtggcttcctggatgacccctgctgtgattctttgacctgccagctgaggccaggtgcacagtgtgcatctgacggaccctgttgtcaaaattgccagctgcgcccgtctggctggcagtgtcgtcctaccagaggggatgacttgcctgaattctgcccaggagacagctcccagtgtccccctgatgtcagcctaggggatggcgag

SEQ ID NO: 73 - DNA, 231 bp, Artificial Sequence
  Description of Artificial Sequence Synthetic polynucleotide
ggagaagagtgtgattgtggcatcatgtatctggacacctgctgcaacagcgactgcacgttgaaggaaggtgtccagtgcagtgacaggaacagtccttgctgtaaaaactgtcagtttgagactgcccagaagaagtgccaggaggcgattaatgctactaaaggcgtgtcctactgcacaggtaatagcagtgagtgcccgcctccaggaaatgctgaagatgacact

SEQ ID NO: 74 - DNA, 231 bp, Artificial Sequence
  Description of Artificial Sequence Synthetic polynucleotide
aatgaagaatgtgactgtggtaataaaaatgaaaagaagtgctgtgattataacacatgtaaactgaagggctcagtaaaatgtggttctggaccatgttgtacatcaaagtgtgagttgtcaatagcaggcactccatgtagaaagagtattgatccagaggattttacagagtactgcaatggaacctctagtaattgtgttcctgacacttatgcattgaatggccgt

SEQ ID NO: 75 - DNA, 222 bp, Artificial Sequence
  Description of Artificial Sequence Synthetic polynucleotide
ggggaagagtgtgactgtggagaagaagaggaaccctgctgcaatgcctctaattgtaccctgaggccgggggcggagtgtgctcacggctcctgctgccaccagtgtaagctgttggctcctgggaccctgtgccgcgagcaggccaggcaggacctcccggagttctgtacgggcaagtctccccactgccctaccaacttctaccagatggatggtacc

SEQ ID NO: 76 - DNA, 222 bp, Artificial Sequence
  Description of Artificial Sequence Synthetic polynucleotide
ggggaggaatgtgactgtggaaccatacggcaggatccctgttgtctgttaaactgtactctacatcctggggctgcttgtgcttttggaatatgttgcaaagactgcaaatttctgccatcaggaactttatgtagacaacaagttggtgaagaccttccagagtggtgcaatgggacatcccatcaatgcccagatgatgtgtatgtgcaggacgggatc

SEQ ID NO: 77 - DNA, 222 bp, Artificial Sequence
  Description of Artificial Sequence Synthetic polynucleotide
gaagagcagtgtgactgtggatccgtacagcaggacgcctgttgtctgttgaactgcactctaaggcctggggctgcctgtgcttttgggctttgttgcaaagactgcaagttcatgccatcaggggaactctgtagacaagaggtcaatgaagaccttccagaatggtgcaatggaacatctcatcagtgtccagaagatagatatgtgcaggacgggatc

SEQ ID NO: 78 - DNA, 225 bp, Artificial Sequence
  Description of Artificial Sequence Synthetic polynucleotide
ggagaggagtgtgattgtggaaccccggccgaagaaggagcagagtgttgtaagaaatgcaccttgactcaagactctcaatgcagtgacggtctttgctgtaaaaagtgcaagtttcagcctatgggcactgtgtgccgagaagcagtaaatgatgatattcgtgaaacgtgctcaggaaattcaagccagtgtgcccctaatattcataaaatggatggatat

SEQ ID NO: 79 - DNA, 222 bp, Artificial Sequence
  Description of Artificial Sequence Synthetic polynucleotide
ggggaggagtgtgattgtggttttcatgtggaattatgctgtaagaaatgttccctctccaacggggctcactgcagcgacgggccctgctgtaacaatacctcatgtctttttcagccacgagggtatgaatgccgggatgctgtgaacgaggatattactgaatattgtactggagactctggtcagtgcccaccaaatcttcataagcaagacggatat

SEQ ID NO: 80 - DNA, 222 bp, Artificial Sequence
  Description of Artificial Sequence Synthetic polynucleotide
ggagaggactgtgattgtgggacatctgaggaaatttgctgtgatgctaagacatgtaaaatcaaagcaacttttcaatgtgcattaggagaatgttgtgaaaaatgccaatttaaaaaggctgggatggtgtgcagaccagcaaaagatgaggacctgcctgaaatgtgtaatggtaaatctggtaattgtcctgatgatagattccaagtcaatggcttc

SEQ ID NO: 81 - DNA, 222 bp, Artificial Sequence
  Description of Artificial Sequence Synthetic polynucleotide
ggagaagagtgtgactgtggacctttaaagcatgatccctgctgtctgtcaaattgcactctgactgatggttctacttgtgcttttgggctttgttgcaaagactgcaagttcctaccatcagggaaagtgtgtagaaaggaggtcaatgaagatcttccagagtggtgcaatggtacttcccataagtgcccagatgacttttatgtggaagatggaatt

SEQ ID NO: 82 - DNA, 222 bp, Artificial Sequence
  Description of Artificial Sequence Synthetic polynucleotide
aatgaggaatgtgactgtggttccacagaggaggatcggtgttgccaatcaaattgtaagttgcaaccaggtgccaactgtagcattggactttgctgtcatgattgtcggtttcgtccatctggatacgtgtgtaggcaggaaggaaatgaagaccttgcagagtactgcgacgggaattcaagttcctgcccaaatgacgtttataagcaggatggaacc

SEQ ID NO: 83 - DNA, 228 bp, Artificial Sequence
  Description of Artificial Sequence Synthetic polynucleotide
aatgaaatctgtgattgtggtactgaggctcaagcaagctgttgtgattttcgaacttgtgtactgaaagacggagcaaaatgttataaaggactgtgctgcaaagactgtcaaattttacaatcaggcgttgaatgtaggccgaaagcacatcctgaagacatcgctgaaaattgtaatggaagctcaccagaatgtggtcctgacataactttaatcaatggactt

SEQ ID NO: 84 - DNA, 222 bp, Artificial Sequence
  Description of Artificial Sequence Synthetic polynucleotide
ggcgaggagtgtgactgcggccctggccaggagctctgctgctttgctcacaactgctcgctgcgcccgggggcccagtgcgcccacggggactgctgcgtgcgctgcctgctgaagccggctggagcgctgtgccgccaggccatgggtgacgacctccctgagttttgcacgggcacctcctcccactgtcccccagacgtttacctactggacggctca

SEQ ID NO: 85 - protein, 230 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGTEQCDCGSLHACCDENCILKAKAECSDGPCCHKCKFHRKGYPCRPSSRSDLPEFCNGTSALCPNNRHKQDGS

SEQ ID NO: 86 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTEQDIGETCCDIATCRFKAGSNCAEGPCCENCLFMSKERMCRPSFEEDLPEYCNGSSASCPENHYVQTGH

SEQ ID NO: 87 - protein, 245 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAENLYFQGTEQCDCGYKEAKKCCNPADCTLVRSAECGTGSCCNNKTCTIHERGHVCRKSVDMDFPEYCNGTSEFCVPDVKAADLE

SEQ ID NO: 88 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGREECDCGSFKQSYCCQSDCHLTPGSICHIGECCTNCSFSPPGTLCRPIQNIDLPEYCHGTTVTCPANVYMQDGT

SEQ ID NO: 89 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGPAQEPCCDAHTCVLKPGFTCAEGECCESCQIKKAGSICRPAKDEDFPEMCTGHSPACPKDQFRVNGF

SEQ ID NO: 90 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGPPEDRCCNSTTCQLAEGAQCAHGTCCQECKVKPAGELCRPKKDMDLEEFCDGRHPECPEDAFQENGT

SEQ ID NO: 91 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTPKEDPCCEGSTCKLKSFAECAYGDCCKDCRFLPGGTLCRGKTSEDVPEYCNGSSQFCQPDVFIQNGY

SEQ ID NO: 92 - protein, 239 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGYSDQECCFDANQPEGRKCKLKPGKQCSPSQGPCCTAQCAFKSKSEKCRDDSDAREGICNGFTALCPASDPKPNFT

SEQ ID NO: 93 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGSVQEAGGNCCKKCTLTHDAMCSDGLCCRRCKYEPRGVSCREAVNEDIAETCTGDSSQCPPNLHKLDGY

SEQ ID NO: 94 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGEPEERCCNATTCTLKPDAVCAHGLCCEDCQLKPAGTACRDSSNSDLPEFCTGASPHCPANVYLHDGH

SEQ ID NO: 95 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEQCDCGFLDDPCCDSLTCQLRPGAQCASDGPCCQNCQLRPSGWQCRPTRGDDLPEFCPGDSSQCPPDVSLGDGE

SEQ ID NO: 96 - protein, 234 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGIMYLDTCCNSDCTLKEGVQCSDRNSPCCKNCQFETAQKKCQEAINATKGVSYCTGNSSECPPPGNAEDDT

SEQ ID NO: 97 - protein, 235 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGNEECDCGNKNEKKCCDYNTCKLKGSVKCGSGPCCTSKCELSIAGTPCRKSIDPEDFTEYCNGTSSNCVPDTYALNGR

SEQ ID NO: 98 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGEEEEPCCNASNCTLRPGAECAHGSCCHQCKLLAPGTLCREQARQDLPEFCTGKSPHCPTNFYQMDGT

SEQ ID NO: 99 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTIRQDPCCLLNCTLHPGAACAFGICCKDCKFLPSGTLCRQQVGEDLPEWCNGTSHQCPDDVYVQDGI

SEQ ID NO: 100 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQEEQCDCGSVQQDACCLLNCTLRPGAACAFGLCCKDCKFMPSGELCRQEVNEDLPEWCNGTSHQCPEDRYVQDGI

SEQ ID NO: 101 - protein, 232 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGTPAEEGAECCKKCTLTQDSQCSDGLCCKKCKFQPMGTVCREAVNDDIRETCSGNSSQCAPNIHKMDGY

SEQ ID NO: 102 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEECDCGFHVELCCKKCSLSNGAHCSDGPCCNNTSCLFQPRGYECRDAVNEDITEYCTGDSGQCPPNLHKQDGY

SEQ ID NO: 103 - protein, 231 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLENLYFQGEDCDCGTSEEICCDAKTCKIKATFQCALGECCEKCQFKKAGMVCRPAKDEDLPEMCNGKSGNCPDDRFQVNGF

SEQ ID NO: 104 - protein, 241 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMAENLYFQGEECDCGPLKHDPCCLSNCTLTDGSTCAFGLCCKDCKFLPSGKVCRKEVNEDLPEWCNGTSHKCPDDFYVEDGI